SEQUENCE LISTING (10 sequences)

SEQ ID NO: 1 - protein, 39 aa, unidentified
  REGION 1..39: note = Signaling or targeting protein
VTMNAPMTSVAPPPSVLSQEPRLLISGMDQPLPLRTDLI

SEQ ID NO: 2 - protein, 540 aa, Homo sapiens
MPFAEDKTYKYICRNFSNFCNVDVVEILPYLPCLTARDQDRLRATCTLSGNRDTLWHLFNTLQRRPGWVEYFIAALRGCELVDLADEVASVYQSYQPRTSDRPPDPLEPPSLPAERPGPPTPAAAHSIPYNSCREKEPSYPMPVQETQAPESPGENSEQALQTLSPRAIPRNPDGGPLESSSDLAALSPLTSSGHQEQDTELGSTHTAGATSSLTPSRGPVSPSYSFQPLARSTPRASRLPGPTGSVVSTGTSFSSSSPGLASAGAAEGKQGAESDQAEPIICSSGAEAPANSLPSKVPTTLMPVNTVALKVPANPASVSTVPSKLPTSSKPPGAVPSNALTNPAPSKLPINSTRAGMVPSKVPTSMVLTKVSASTVPTDGSSRNEETPAAPTPAGATGGSSAWLDSSSENRGLGSELSKPGVLASQVDSPFSGCFEDLAISASTSLGMGPCHGPEENEYKSEGTFGIHVAENPSIQLLEGNPGPPADPDGGPRPQADRKFGEREVPCHRPSPGALWLQVAVTGVLVVTLLVVLYRRRLH

SEQ ID NO: 3 - protein, 540 aa, Homo sapiens
MPFAEDKTYKYICRNFSNFCNVDVVEILPYLPCLTARDQDRLRATCTLSGNRDTLWHLFNTLQRRPGWVEYFIAALRGCELVDLADEVASVYQSYQPRTSDRPPDPLEPPSLPAERPGPPTPAAAHSIPYNSCREKEPSYPMPVQETQAPESPGENSEQALQTLSPRAIPRNPDGGPLESSSDLAALSPLTSSGHQEQDTELGSTHTAGATSSLTPSRGPVSPSVSFQPLARSTPRASRLPGPTGSVVSTGTSFSSSSPGLASAGAAEGKQGAESDQAEPIICSSGAEAPANSLPSKVPTTLMPVNTVALKVPANPASVSTVPSKLPTSSKPPGAVPSNALTNPAPSKLPINSTRAGMVPSKVPTSMVLTKVSASTVPTDGSSRNEETPAAPTPAGATGGSSAWLDSSSENRGLGSELSKPGVLASQVDSPFSGCFEDLAISASTSLGMGPCHGPEENEYKSEGTFGIHVAENPSIQLLEGNPGPPADPDGGPRPQADRKFQEREVPCHRPSPGALWLQVAVTGVLVVTLLVVLYRRRLH

SEQ ID NO: 4 - protein, 540 aa, Homo sapiens; identical to SEQ ID NO: 3

SEQ ID NO: 5 - DNA, 29325 bp, Homo sapiens
  source 1..29325: mol_type = genomic DNA
acatggccaatggccgcgcgctctgcccgccccgcctcctcgctgcgggaagggtcctgggccccgggcggcggtcgccaggtctcagggccgggggtacccgaggtaagatcgcttcccgggcgttgggtcctttcggctcagcacgcacggacgcctttagggaaggtggctgcagcggcaggacggagtccgccgggacgccctgggtctggggtgcgcggggggcccaggaggggacaggacgcgcggggatccggaagagcgggccctgtcgcaagagtttcgggaacactgagggctccaggcgccgcatccagcatccggggaaaagggggtaggtggcgctgggcgtctgctcaggctgggggaaaggtagggccagaaggggacgggcagcggccgctgacctcctcctgccgcccgcgggcccagggtgacgctaaggtggggccgagcctcgaccgggtgcgcctagaggtcgagtgctgccgccctccgctgggtctggacagttctcggcggcgacaccagctcaaaacggcctccccgccctccgcggacctgggtcgcgcccaggaatccgatccaaggctgtgaggcctgtccctttgggaagggtgggtgtttatttccgggatgcactcagagcctctggacagtcaggtcggaaactttgctgtattgggaacactctgtcacctacttccttctcagttgggaaggaagtgccaagaaaacatgaaacaaaccaaaaacacgaaaaagggattctctgtatggaagccgtgaagcctcaaaaatatctaggaggacagccagcgacctgggacctgtggcagccatgtgaaagcagggttaatgtctggactaaatgttgcttccacctaagtgcaccctcagcctccctcccgccaagtgaccttgggtcctctttgggcttgaaggcaggtggctgtgtgggtctcgctgcaggggtctctgtgccctgcaaggtgtatgaccagttgcagtgaggcagcaggttttgggttcaaatcctgtgctagcctctggccagctgtgcaccttggcaacactttccctgtcacgggattggggaaggattaactgaaagaaccttaggatgtgcctgtctacaatgggccctccataaatgtgaacaaatgtgggcttcctttccttttgtttgggccacatcatcccttcccctccatctgtggctgaagctggaatgcagaagagtgcctcatctgactgccttctggtacctggctgatgccatgagaaaggaaggagaaaggggtctttttttttttgaaatggagtctcactctcttgcccaggctggagcgcagtggtccaatcttggctcactgcaacctctgcctcctgggtctagagattctcctgcctcagcctcctgagtagctgggactacaggtgtgtgccaccatgcctggctaatttttgcatttttagtagagacggggtttcaccatgttggccaggctggtctcaaactcctgacctcaagtgatctgcctgcctcggcctcccaaagtgctgggattccaggcgtgagccaccgcgcccggccgagaaagggatctattaactcccatagagttgttctttgctaatttcttgaaggctcagaggacccccgcctcaccttcctgattctcctgacctgtcattagtacttgccccacgaggaatgtagcagggcctgctggctggcaaagcaactcatgcatgtgaggctctgaggccagtgacaggactgcttcccctgtgaggaaggtctggtggcccaacagcttttaggtgctgtctgctctacagcactgcctcctgagagaggtctcatgcctgcctgatgcccacttggtcctctcctgcctccttccctccctgacaacccacttggaatccaatagcatctcaaacttcacttgttccgaactgagttctggagtcccttctgagccactgctcctcccctggcttctctggcctggtaaaagggcaccttccatccacccagtgcccaaggagtcatgcttcttttctctcccttatctcctacaccctcaaaacaccaggaatctggctgcctcctgccatctctgtggttcccatcctgaccatagtcatcctgtctcctgggctgtggcctccttactggtctcccagttttcatcctggcccctccaaagtcctcacaaccaccagagaagtctttaatgtaaatcagatcctcttctttccctgccggaaccttccagtggttccctgtttcactccaactaaaacccagagtcctttcctacagcactctacatgagtggcccctgccacctccttgaccttgacaatctctgcccctttccctagcttgcttgcttttttttttttttttcctatgatggagttttgatcttgtcacccaggctggagtgcaatgggatgatttcagctcactgcaacctctgcctcctgggttcaagcgattctcctgcctcagcctcccgagtagctgggattacaggcgcccaccaccatgctaatttttgtatttttagtagagaaagggtttcaccatgttggcgaggctggtctcaacctcctgagctcaggtgatccacccgcctaggcctcccaaagtgctgggattataggcgtgagtcacgcagccagccatccctagctttcttgacctagaccacactgacctgctttctattcttcaaacatgccaagctcattcttgttttaggacttttgcatttaccatgctctctgcctaaaacaccaatcttctcagagcctgagaacagctcagctgtgttctgcacgctagttcagaaaggcttctttgaccccctagttcaagtagcatgcctgtctccaggggctctgtctcattacctgctttactttctttagagcctttattgctatctggaactcttatttgggtattaatttactgatctgttatttgtcccaccccattagaatataaattggatgtggcatagaccttgtctcttttattccctgcagcactccctgatgggggcggcagaaaagtaacgagcaaatacatctataactgcaaattgtggtaactcctacataaacaactggcagcaattgcttatatttgaggcacttaaaaatttttaagctggctgggcgcggtggctcatgcctgtaatcccagcactttgggaggccgaggcgggcagatcacgaggtcaggagatcgagaccatcctggctaacacggtgaaacctgtctctactaaaaatacaaaaaattagccgagcgtggtagcaggcgcctgtagtcccagctacttgggaggctgaggcaggagaatggtgtgaacccgggaggcggagcttgcagtgagccgagattgcaccactgcactccagcctgggtgaaggagcgagactgtctcaaaaaaaaaaaaaaaaaaaaaaaaagaaatttttttaagctgctgggcgcggtggctcacgcctgtaaatctcagcactttgggaggccgaggtgagcggatcacctgaggtcgggagttcgagaccggaacatggtgaaaccctgtctctactaaaaatacaaaattagcggggcgtggtggctcatgcctgtaatcccagctacttgggaggctgaggcaggagaatcgcttgaacccaggaggcagaggttgcagtgagccgagatcgcgccattgtactccagcctgggcaaaaagagtgaactccatttcaaaaaaaaaaaaaaaggccaggcgcagtggctcacgcctgtaatcccagcactttgggaggccgaggcaggcggatcacgagttcaggagattgagaccatcctggctaacacggtgaaaccctatctctactaaaaatacaaaaaattagccgggtgtggtggcgggcgcctgtggtcccagctactcgggaggctgaggcaggagaatggtgtgaacccaggaggtggagcttgcagtgagccgagattgcaccacagcactccagctttggtgacagagcgaaactccgtctcaaaaaaaaaaaaaaaaaaaatttaagcttagaggccggccacagtggctcagcactgtgcaggccaaggcaagaggatcacttgaggtcaagagttcgagaccagcctggccaacatggtgaaaccctgtctctactaaaaaatacaaaaattggccaggcgcgttggctggcgcctgtaatcctagcaacttgggagaccaaggcaggcagatcacctggggtcaggagttcaggaccggcctggccaacattaaaacatataaaaccccgtctctactaaaaatataaaaattatccaggcatggtggcgtgtacccgtaatcccagctactcgggaggctgaggtaggagaattgcttaaacccgagaagcagaggttgcagtgaaccgagattacgccactgcactccagcctgggcaacagagcgagactctttctcaaaaacaacaacaacaacaaacaaacaaattagccaggcatgatggtgggcacgtgtaatcccagctactcgggaggctgaggcaggagaattgcttgaatgtgggagatggaggctgcagtgagccgagatcacaccactgcactccagcctgggcgacagggagactctgtctcaaaaaaaaaaaaaaaaaaaaaagtttataaggctgaattaccgtactgtcaaaacaagctgctatctgagccgttttaagggtgaggaagtctggaaactgataacttgcccaggacacacagtgagttcaaggcatggaactcagtctcctatcttaagaatgtatgtgggccgggcatggtggctcacgcctgtaatcccagcgctttgggaggccaaggcaggcagatcatctgaggtcaggagttcaagaccagcctgaccaacatggagaaaccctgtctctactaaaaatacaaaattaaccaggtgtggtggtgcatgtctgtaattccagctactcaggaggctgaggcagaagaatcacttgaacccggaaggcagaggttgcgatgagccgagattgtgccattgtactccagcctgggcaacaagagtctggaactctgtctcaaaaaagaaaaaaagaatgtatgtgtagcaggctttttttttttttttttcccccgagacggaatctggctctgtcgcccaggctggagtgcagtggcgcaatcttggctcactgcaagctccgcctcccaggttcacgccattctcctgcctcagcctcccgagtagctgggactacaggcacccgccagtacgccgggctaattttttgtatttttagtagagacggggtttcaccgtgttagccaggatggtcttgatctcctgacctcgtgatccacccgcctcggcctcccaaagtgctgggattacaggcgtgagccaccgtgcccggcctatgtgtagcaggctttaatggtgggcctgcagccatgtcatggaaagaagctgacctgaagatctcagttctttcttcttctactaactagcaagcatacctcagtttcttctttaaagcgggatgatccgattattatcatgttggggttcactttttattttttcagtgtgtcccaaagcagcagcacgtttaggtatagccctcttgctatcagcttgagggccttagagccaggaagggagccaggacatttataggcacagaaactagggtcacatacagatccccccaccgcatgtgctaggggtacatgcagaccttcccagtgctgaccaacctgcagagaagaaatgggccctaggtattctggatctgattctttttggtcttcaattatttttatttttatttttttagagacagggtctcgctgtgttgcccaggctggcctcgaacagctgggctcaagcgatcctcctgccctagcttcttgagtagctggtggtcatcaattcatttttagcaaattctgcagaatttttttttttttttttttttttgagacggagtctcactctgccgcccaggctggagtgcagtggcgtgatctcggctcactacaacctccgcctcttgggttcaagcaattctctgtctcagcttcctgaatagctgggactgcaggcgcccgccaccatgcttggctaatttttttgtattttcagtagagacggggtttcaccatcttggccaagttggtattgaactcctgacctcgtgatccatccgcctcggcctcccaacgtgctggggttacaggcgtgagccaccgcgcccgggttctgcaggaattttggagagactcaggcagtaataaaataggatgtttacagaaattaaagatggcggccgggcgcggtggctcacgcctgtaatcccagcactttgggaggccgaggcgggcgcatcacgaggtcagtaaatcgagaccatcctggctaaccccgtgaaaccccgtctctactaaaatacaaaaaaattagccgggcgtggtggcgggcgcctgtcgtcccagctactcaggaggctgaggcaggagaatggcgtgaacccgagaggcggagcttgcagtgagccgagatcgcgccaccgcactccagcctgggcgacagagaaagactccgtctcaaaaaaaaaaaaaagaaattaaaggtggctggacacattggctggtgcttgtcatccgagctacttgacaggcggaggcagggggatcgcttgaggccaggcgtttgagaccagcctgggcagcatcatgagaccctgtctctagaaaaaataaaaaaattagctgggcatagtggcgcaggtttgtagttccagctaccggggatgctgaggcgggaggattgcttgagcccacgagttcgaggctgcagtgaactattattgcaccactgcacccaacttgggtgacagagaccccatctgtttgtttgtttgtttttgagacagagtttcgctcttgttgcccaggctggagtgcaatggtgcaatcttggctcaccgcaacctctgcccccaggttcaagcaattctcctgcctcaacctcccgagtagctgggattacaggcatgcgccaccatgcccagctattttttttttttttttgtatttttagtagagacgggattttctccatgttggtcagtctggtctccaactcccgacctcagttaatcccccaaattggcctcccaaagtgctgggattataggcgtgaaccactgtgcccagcccgagaccccatctcttaaaaacaaaataaaacaaaacaaaaacggccaggtgtggtggctcacacctgtaatccccaaacttgggaggccgaggcgggtggaccacttgaggtcaggagtctgtgaccagcttgccaacatggtgaaaccccatctctactaaaaatacaaaaattagctgggcatggtggtgcgcacctgtaatcccagctactcagaagggaggctgaggcaagagactcaattgaacccaggaggcggaggttgcagtgagccgagattgccccactgcactccagcctgggtgacaaagtgagactcgctctcaaaaaaaaaaaaaagaagaaattaaagatgaaagaaaacaaacattccaaaaagttgagaaagaattgccttttgtccagccccactcccaaccccccaaccctgttgtaatgtgtgatctgttttcttccagtctcgtttcctctcagtccatccacccttcatggggccagagccctctctccagaatctgagcagcaatgccgtttgctgaagacaagacctataagtatatctgccgcaatttcagcaatttttgcaatgtggatgttgtagagattctgccttacctgccctgcctcacagcaagagaccaggtgagcaagggaagtgacagcccgacactggcctgggggcagggctgtggaattcaaagctcagccccatcctagttcctcacccaagcctgggctggctccttccttcttcctcttgctgtgtcttgctccttgtccttgctgcttttcttttttttttttttttttgagattgagtctcgttctgtcgccaggctggagtgcagtggcacgatcttggctcattgcaacctccgcctcctgggttcaagtgattctcctgcctcagcctcctgagtagctgggattacaggtgcgtgccaccacgcccagctaatttttttgtttttaatagagacggggtttcaccatgttggccaggatggtcttgatctcttgaccttgtgatccgcctgcctcggcctcccaaagtgctgggattacaggcgtgagccaccgcacccttgctgcttttctaacttttggatggagtgtggctcagggtggcgttgctgacttcgccgagctcccccttgtgttgcttttgtgcactgctcaaaaatatggcgctggctctctgagatttcctggctctggtccacttgcccactttttttggaacctcctatttccttcatctctcttgcccttccttgtcctgctcagttttgattccattctccttgtcatggggccctgtcctggcacggagctgggactcaggtttgagagctggcaggatcagggtcgctctagccccaacagaacttgctgcaggcccctggcactcactagctggtgaaacgggcacaacccctccccgttgtagctgctgttctcagattggacccctgtgctccagagggtacctgttggctcttttggggcctcctgtcctcagatttctcaggagccccattgttgtctccgctgtcctcccacacagatcgcattagtatgcaggtctgtttggagtttgctcctccctcttgtattttggggtttatagggatatcttgttttatagtaaatattttctgtgggttttctttattttctttaaaaaatttttttttgagacggagtctcgctgtgttgcccaggctgcagtgcaatggcatgatctcagctcactgcaacctctgcctcctgggttcaagtgattctcgcgcctcagcctcctgagtagctggggttacaggcgcatgccaccacacctggctgattttgtatttgtagtagagatggagtttcaccatgttggccaggctggtctttatttttatttttgagacagagtcttgctctgtcactcaggctggagtgcagtggcacgatttttttttttttttgagacggagtctcactctgtcgcccaggctggagtgcagtggtgtgatctcggctcactgcaagctctgcctcctgggttcacgccattctcctgcctcagcctcttgagtagatgggactacaggcgcctgccaccatgcccggctaattttttgtatttttaatagagacggggtttcactgtgttagccaggattgtctcgatctcctgacctcatgatccacccgcctcggcctcccaaagtgctgggattacaggcgtgagccactgcgcccagcattttttttttttttttttttgagatggagtctcgctgtgtcttccaggctggagttgcagtggtgccatcttgggtcaacctctgcctcctgggttcaagcaattctcctgcttcagcctcctgagtagctgggattacaggtatatgctaccacacccggctaatttttgtgtttttagtagagacggactttcaccatgttggtcaggctggtcttgaactcctgaccttgtgatcctcggccttccaaagtgctgggattacgggtgtgagctaccgcacctggctattttcctttttctaaaaatctagctcctgcaggattctgtgggtttttgtttctgctgtctggttgcttgtttttatgtgagaattcaggtagacataaaaactctagggctgggcacggtggctcacgcctgtaatcccagcgctttgggaggccaaggcgggtggatcacctgaggtcaggagttcgagaccagcctggccaacatggcgaaaccatgtctctactaaaaatacaaaaaaattagccgggtgtggtggtgggctcctgtaatcccagctactcgggaggctgaggcaggagaatcgcttgaactcaggaggcagaggttgcagtaagctgatatcacggcactgcactccagcctgggcgacggagtgggactccgtctgaaaaaaaaaaaaaaaaaagaaacaaaaaaactctgcagccactgtcatctgcccacaatctccccagcattctcagcttccttgtttgttattgtcggccccctctctttccgtcttttgcccctttcatcatacttttgctatctaccttttccttctctcctaatccaaacctttctttttgccctgggggccatattaatccaaggcttttgtatcagattaactgggtttggattcctgccccactgttttaggatctttgctacagtactttgcttctgctaagcctcagtttcctcattagtaaagtggagataataatggcattaaataaagatgatacatgcaaagcccttaatggagagcccaggacatagttaattgccagtttccggcagctgcctttattgatgtggctgctaattgctcttcctcactccatacctggccctgtcctgggctccgatccagtttcacgtggctgccttgcccttgtggctttcttggcacccctccccccgctgtggcttcattctgggtggggaagtggcaggggccacctggcttgagcaggacagtggcattgtgtcttccaggatcgactgcgggccacctgcacactctcagggaaccgggacaccctctggcatctcttcaatacccttcagcggcggcccggctgggtggagtacttcattgcggcactgaggggctgtgagctagttgatctcgcggacgaagtggcctctgtctaccagagctaccagcctcgtgagcgtcctgcccttgccctcctggacccccagcctgctccctggcctccgctctccttttctctctccctgtacttcctgcctttctctgtcatcctctttcttgtcactgtgaagcgatgaataaacctgggtgtagatccaggctgagccacttaccagctgtgtccctttggccaagtcccttaatttccctgagcctcaggcctctcttctgtaaaatgaagctcatggcagcatctgccgcggggagctgcagtgggtgatactgcgggacgatgcgtgttgagtattgagctgggctgggcacttcctgtatgcccagcacatggagtctcccctaactttcacggctgtagcattcgcctcccacccttcctcatttcttctcccccacctactcattcaccctccctctctcctccttctcttcccctcccctggtttaccctgagagccttcgacgccctctatcagctgcccagttattctttaagtccctctcagtgtccctgccactctgagtgctcggaggcgatttgatgagattgagtttgatcctgagtgagatcaagacatgggaggaggctgggcgcggtgtttcacacctgtaatcccagcactttgggaggccgaggcaggcggatcatgaggtcaggagatggagaccaccctggctaaaacagtgaaaccccgtctctactaaaaatacagaaaattagccgggcatgttgtcccagctactcaggaggccgaggcaggagaatcacttgaaccagggaggcagaggttgcagtgagctgagatcgcgccactgcactccagcctgggcgacagagtgggattccatctcaaaaaaaaaaaaaaaaagacatgggaaaaaaaatcaagccagccctatttatatttcaaactagaggtaacccccgagaccctggtcacatttatagctgtgggacatccatgtttttcttttctttctctctcttttttttttttccttttagagacagagtcttgctgcgccacccaggctgcagtgcagtggtgcaatcatagctcactgcagccttgacctcctggactcaagtgatccttctacctcagcctccagagtagctgggactacaggcatggacaactacacctggctaatttttaaattttttgtagagatgacatctcactatgttgcccaggctggtctcaaactcctgggctgaagcgatcggcctcccagagtgctgggatcataggtgtgagccaccgcgtctggctctcatgcttgcttttctctcctttttcccttccttgcttttcctccctccctccctcccttcctctcttccttcctttttttccttccttctttttaaatatgtctcttcatgtgtggagattaatagtgatccctggctgggcacggtggctcacgcctgtaatcccagcactttgggaggccgaggcgggcggatcacaaggtcaggagttcgagaccagcctggccaatatggtgaaaccctgtctgtaccaaaaatacaaaaaaattagctgcgcatggtggtgcaagcctgtaatcccagctacttgggaggctgaggcaggagaattgcttgaaccggggaggtggaggttgcagtgagccgagattgcgccactgcactccagcctggatgacagagtgagactccgtctccaaaaaaaaaaaacccaaaaatagtgatcccctgaatacaatggctgtggtagggcctgatgaggggtgggggcaaaggggaggggctcaggtggcagcatcagggcaggggtcagtgagcaatgatagtcatgtggaggagaaagccactgggtcctaggatgcctggggacagagaagagtgactgctgacacggcgtgggtgactagagacccacgaggcccccccatactccccttcctcccttgctaccttgtcctccatctgctctcaccctcccactcctgcccccttgccaagtgatgcttgtcactccttttttttttgaaatggagtttcgctctgtcgcccaggctggagtgcagtggtgccatctcagctcactgcaagctccgcctcccgggttcacgccattctcctgcctcagcctcccgagtagctgggactacaggcgcctgcaaccatgcccggctaactttttgtattttttagtagagatggggtttcaccgtgttagccaggatggtctcgatctcctgacctcgtgatccacccgcctcggcctcccaaagtgctgggattacaggcgtgagccaccaagcccagccctgcttgtcactcttgaggagtgggcccacatcagaacagcttttggacctatgggtggggcggggggtgtacccaagagcacccaagcctctttaatcatgaggagaacccccaattcctttttttttgagacagagtcttgctcagtcgcccaggctggagtgcagtggcatgacttcggctcaccacaacctctgcctcccgggttcaagtggttctccttcctcagcctccctatagtccctgattccttctattttttttttttttttttgagacggagtctcgctcttgttgcccaggctggagtgcaatggtgcaatctcaggtcatggcaaccttcacttcccaggttcaagcaattctcctgcctcagcctctcgagtagctgggattacaggcatgcgcctccacgcctggctaattttgttatttttagtagagacagggtttctccatgttggtcaggctggtctcgaactcacgacctcaggtgatccacccacttcggcctcccaaagtgctgggattacaggcgtgagccaccacgtctggcttctttttctttttttcccccgagacggagtcttgctctgttgcccaggctggagtgcagtggcgcgatctcagctcactgcaacctccgtctcccaggttcaagcaattcttctgcctcagcctcctgagtagctgggattacaggtgcttgccagcacgcctggctaatttttgtatttttagtagagacggggtttcactatgttggccaggctggtcttgaactcctgacctcctaatccacctgccttggcctccccaaatcctgggattacaggcatgagccatcgtgcccagcccctgattccttctttttttttctttctttttttttttagacggagtctcgctctgtcgcccaggctggagtgcagtggcgcgatcttggcttactgcaagctccgcctcccgggttcacgccattctcctgcctcagcctcctgagtagctgggactacaggggcccgccaccatgcccggctaataataatgttgtatttttagtagagatggggtttcactgtgttagccagggtggtctcgatctgacctcgtgatctgcctgccttggcctcccaaagtgctgagattacaggcatgagccactgtgcccagccctgattccttcttgatatcactacatctttgtcctctagggacctcggaccgtcccccagacccactggagccaccgtcacttcctgctgagaggccagggccccccacacctgctgcggcccacagcatcccctacaacagctgcagagagaaggagccaagttaccccatgcctgtccaggagacccaggcgccagagtccccaggagaggtctgtcctcatagtctaccttgagccaccacttttgtgttcctatctgcccacttctgcccattgagccttccagaaaccctctcccgtcccctataaatcacgcctaatctctgctcagaaccctagggcttcctcagtggggatctgccccagaccagcttccaggctgctgaccaggtcttcaccctgtggcagccctaatcctctgtcagcaaccagctgggagaccacagttttgtgtgtgtgtgtgtgtgtgtgtgtgacagtgtctcattctgtcacccaggctggagtgcagtggagtgatcttggctcactgcaacctctgcctcctgggttcaggtcattctcctgcctcagcctcctgagtagctgggattacaggcacccaccaccacgcccagctaatttttgtatttttagtagagatggggttttgccgtgtcagccaggctggtctcgaactcctgacctcaggtgatctgcccacctttgcctcccaaagtgctgggattacaggcgtgagccaccgcacctggcaatgctgtgtgttttctgtgaggtagacgtaaggacacctgtggacagagggtctgggaattaccagaacccaggcaagggctcccctggctcctgtgctccatggtgtgggctgaggcctataggagatgccccaagagcacaagctgccctttgtgagctcttgggagaggcaactgccttattcatattttccctcattgcagaattcagagcaagccctgcagacgctcagccccagagccatcccaaggaatccagatggtggccccctggagtcctcctctgacctggcagccctcagccctctgacctccagcgggcatcaggagcaggacacagaactgggcagtacccacacagcaggtatgcatggaatctggaattatagggtccttctgatctctcaagtgagggtaagaattagagttgccccatctggcttccttgaacaggagacaaggtgggaataaagggagttcaacccaggaagcaaaccagttccttagtgggtgtatcagttagcatttgctgtgtaacaaatagtccactccagttttccaaattttttttttagtagcttaaaatacagccatttatttagcatatgatcctgtgggtcaggcatttgggctacctacatgggcatttcttctggtcttggctgaatttcctctcaagtactcaccggtatatacataagttctgcctctggctgtttgctgagcaccttggttctcttctatgtagtctctcatcctccagcacacaaacccatcatggcagctgggcagagttctcagagagggctcaaaactggcacagtgtcccctgtgctccattctgtgggcaaaagcaagttataaggccagcctagattcaaggagtagggaaatagactccctccctagacgggaggactgacaggcacagtgcagtggggctgggtggagatgagcgagataagtagggccatttttgcgctctgccaaagggactgtagggaacagccagggcctatagggcagtgggagagggacagtgaagggctgcatcagctgttggcaggggaacctttaggcactgtcttaccgcagagatctccagttcccagtgaatcatgaaaacttctcagtccccagaggaagtaaggtcttcatcatccagtggcctggactcaactccagatgtcagtgctccccctcagaaatatatagttgtccatctggacctctcaggccagcatgtctctttcctacttcccaaactattccacatgacgctggtgcccagtcagccctcagtgccctgggacagccacaagacacatgagcagttagaggctgggagacgtcatcttagtacttttgtcatccccaaactgctccaagcacctgtctgctttgcagtgtcacctggccacgggatgcctttcaggagttgctgtagaccacagaggcagagggcgcttaggtttcagtacgtttgtagacacaggtcccatgagattctgtggtattagattgtggtgggggagctgtacatcagaatcaccctgacttttgccagctgtggggcttggcatgtgcattccgagttccgtggagagtcctgctgcaactgcctttacagaccatcaccacctgctatcctctgcttcccccacccaggtcaggcagcctcccaggggtggctttgtccttgtcccctctcttcccaagcctccgggatggccaggcctctcggctggtgtgagctgttctgcatgagccatcctgccaccccttgccctgatccatggctgctcccactcatggtggtaggagagggacagcagtgggggaagtgtccaggattgcatgaggctaaggtcaaagtagaaaaggtagacacaggagaggggaggtttcccaggtgggagaggaaaaagcggagagaataattaataatggtcttcaggctcctaggtaccatttcactgtgtgccaggacagacctggggctacaggtcaaggactgagggcagctgttgggctttcaggccaggaagcagtgaccaaagggactgtggcatctcctccaagggcaggagatttggaggcctagacacagtagggaccatgagatctgggccagagggacccttctccaggcctcaaggtaatggtctttgggtctgtgtttccacttgtgtttttccaccggcaggtgcgacctccagcctcacaccatcccgtgggcctgtgtctccatctgtctccttccagcccctggcccgttccacccccagggcaagccgcttgcctggacccacagggtcagttgtatctactggcacctccttctcctcctcatcccctggcttggcctctgcaggggctgcagagggtaaacagggtgcagagagtgaccaggccgagcctatcatctgctccagtggggcagaggcacctgccaactctctgccctccaaagtgcctaccaccttgatgcctgtgaacacagtggccctgaaagtgcctgccaacccagcatctgtcagcacagtgccctccaagttgccaactagctcaaagccccctggtgcagtgccttctaatgcgctcaccaatccagcaccatccaaattgcccatcaactcaacccgtgctggcatggtgccatccaaagtgcctactagcatggtgctcaccaaggtgtctgccagcacagtccccactgacgggagcagcagaaatgaggtgagtcctcgcccttcctggcagggatcctggccccttcccccgggacagcttgcccacctggccctggccttggccccttcccagtctgcattctgtgtccagcctgtgctgctctgtggcctctccttgagggcatacagacagttgagaaccagcctcatgcaggccccacaccatgttctccaggaggaacagtcattgagcttctaagtctggacacctcaggagggtcagccacagggggcacccactggtcaggtgtataagttcatttagggctcgtagttcctagtgaagccgagcggtgccgttttgcacataaggaagcagtgacggggacagcacagtggcccatctgcctcttgccttgctcttcaccaggatgcctggtgtgtccctccatggccaggctttacagaacgcagtcccacctggagcagccactcggacccagcagccccccattgttgcctgctccaagcctcacatctaaccctagctgcggctgtctgctgggaagagccaagtccatagggccctttgggcacatggccaggcctctgaccctgtggctgctctctagttctcaggcccaggcaggatgtcagtgcaggatggagccccgccctaccaaaggcttccaggtgggcatgagctcacaggcaggccagggagtagggaaaggctgccctggaggaggccaccattggtgcagattcttggtcccctctacccccactgctccaagaaaaggtggcctaggggcattatagattgggaattgaggggttggagtgttagttcatgccctggcctgggaatgggaccgccctaccaggttcgtctccctgccaaccccagtcccttccagtgctctcctttctttcccaggagaccccagcagctccaacacccgccggcgccactggaggcagctcagcctggctagacagcagctctgagaataggggccttgggtcggagctgagtaagcctggcgtgctggcatcccaggtagacagcccgttctcgggctgcttcgaggatcttgccatcagtgccagcacctccttgggcatggggccctgccatggcccagaggagaatgagtataagtccgagggcacctttgggatccacgtggctgagaaccccagcatccagctcctggagggcaaccctgggccacctgcggacccggatggcggccccaggccacaagccgaccggaagttccaggagagggaggtgccatgccacaggccctcacctggggctctgtggctccaggtggctgtgacaggggtgctggtagtcacactcctggtggtgctgtaccggcggcgtctgcactagtgaagccctgggctcttcccaccacccatctgttccgttcctgcagtacacctggcccctctccgaagccccttgtccctttcttggggattgtggaggctgggtcagaggggagttaagggactgcaggcctggcagcaggacatgccttggctgaaccaagtcctgagagcagcatctctgtccccacggtgccttgtgtgggtccccgtccttggctttctgggtcctgggctgcccccagtgctccagaccttccccactggcaatccaggttatcatccatgtcctccagaggagcttcctcctccaggcctcagccctgttggcccaggtggagcaggagggaccactggaacatgtggtgcttgggaatgcctctcctgttgcattggtccctgaaggcctcagggcaggtatgtggtgtgtgggcgactccacaagacctgcctcccatcctggcagcccagcctgagaccgttgcattgaggcaggcaggagcggcagggtggctgctctccaggagcccaactgccttgagttcctgccccactgggccccctcccctgctgggcaatcctgggaaggtctggaggttcctgtggacctcagggaagccaggggcagctgtcaggcctgaggaagacctgtggagctcctctccagcctcctctttccctcccctctggtctccattctcttcagctccctacatgggctggggaggagacacctggtgggcagagctcaggcagaggtttggatttcagctccctcacttccggggctgtgtggctttggcagatgtcagacttctggtcttgcttctccacgtggacagtgagtatctggctcattcttcactgggttcttctgagattgaacctacaggtgtttgccaagtgcctggcccagagcaagtggccactgcttctcccatctctctcctgcccaacctggtagagctgagggcatgagaggcagagtgcacagtggtcaagggtgcagctctgcagcacaggcagcctaggcctgcgtcccaacctgcctctcaccagctctgtgaccttgggcaagggatttatctgtctgtcccttagttttctcacctgtaaaaggaggataagtatatatatatatttcccagtgttgtgaagattaaaggagtttatcgatgtaggtcttaggatgagtcctggcatttaccaagggttggatatatgttattatcactattaagtgttgagggtccaggcatgctgggcaacagggaccccatctctacaaaaaagtttaaaaaattagccaggcgtggtggtgcacctgtcgtcttagctacttgggaggctgaggtgggaggatcacttgagcccagaagcttgaagctgcagtgagctaggatcgtgccactgcactccaacctgggtgagagagcgagaccctgtctcaagaaaaagaaaaatgcagagaaacaggagtcttggctactcctttagaggcagactcagaccctcctgcctcacagctttatctttgtatttgccccttactttatcttgtgccttgagaaattgctggggagagaggtatgtccactgggcagctgtacaggatggaggatatagggcgtttccactcccagcagccaggttccctcaccccaagctcacccactgttggggagattatctacaataacaccagaaacacattggggtggattgggggtatccttatgggttcttttcagggaaccattgctggacaaggcacaggagccacctccatttctgagctctgcaagggacaagaactagagccatcaggggctgggctcactgtggccccaccccaagccgtcagcctccagggatctacaccctgccttggctgctacagctttttcactccactgccctaggggagttcagcaacctaatgatctctatctctgaacatctcttcatcccatgctccaagtccagcaacctgcaccctggaaccaggagtggaccctacccgagctgtctgtattaatccccatcccccaccaccaatcttaaaaagccctctgtccccctaccctaaaccccagttaggtacccatgctgggcaggtcagttaacaatttatgcacaggtactagttttattgtattaccgttccagggtagctttgaaaaaagtatctcaaaaaggcaacatgggccgagcgcagtggctcacgcctgtaatcccagcactttgggaggccaaggtgggcagatcgcctgaggtctggagttcaagaccagcctggccaacagggtgaaaccccgtctctacaaaaataagaaaattagccaggtgtagtggcagacgtctgtaatcccagctattcaggaggctgaggcacgagaattccatgaacccaggatgcggaggttgcagtgagccgagattgtgccactgcgctccagcctgggcgacagagtggtattctgtttcaaaaaaaaaaaaaaaggcagtatgtagccccgaagactgttgcccaagtggtagaatgttagcacactaccagcctaggtaaaaaatacaaaaagtaactgggcatggcggcgcccatctatagtcccagctacatgggaggctgaggtgggaagataagtcacttgagcccgccaggaggcggaggttgtagtgagctgagatcgcaccactgcactccagcctgggtgaccgagtgatactctgtctcaaagaaaaaaaattataattttagcacagtaaccagccatgatgggagataccctgggtaaggcatgtagaaagggttgagggaccttcccagtcccctagccccgcctcccatcctcccatctttttcttttttcttttttttagagaatcacccagcctggagcgaagtggtgcaatcataactcactgtatccttaaactcccgggcttaagcgatcctcctgcctcagccttctgagtaactaggacttcaggtacctgtcaccatgcctggctaattaaatttttttttcttttttttttttgagatggagtcttgctctgtcacccaggctggagtgcagtggcgcgatctcagctcactgcgacctccacctcctgggttcaggccattctcccgcctcagcctccagagtagctgggactacaggcgcctgccaccacgcctggctaatttttttgcacttttagtagagacggggtttcactgtgttagccaggatggtctcgatctcctgaccttgtgatccgcccgcctcggcctcccaaagtgctgggattacaggcgtgagccaccgcgcccagccaaattaaattttttatagagatgaggtcatgctgttatgttgcccaggttggcctcatgagatcttgccttagcctcccaaagtgctgggattacagatgtgagacactgcacccaaaccccaccacttttttttttcctttttctttttttgagacagtcttactccgttgcccaggctggagtgtagtggcatgatctcagctcactgcaacctccgcctcccgggttcaagcaattctcctgcctcagcctcccgagtagctgggattacagaggcctgccaccacacccgactaattttcgtatttttagtagagacggggtttctccatgttggccaggctgttcttgaactcctgacctcaagtgctccacctgcgttggcttcccaaagtgctgggatacaggagtgagccactgcgcctggctgatcccagcacttttcaaatgatgccgctcaaagccgtgacttggcctactttgaacagcaaacttgttgctgctgttgtcaacctgaaggcctctcaaatgccagcttcaagcagggtgtgaattggccagtgtcagatctcaggagtcctgtgttgagagtgtggctttcagctgcggggagctgcacttggtggggaaagccaggcaggtcaccctcacagccagataatgtggaggtcagaacccaaggaagggagtgagacctccactcccagtgggggacctggccacccatccttggggacctgagaaagcgtacttcaccttggggtgaaggctgggtggggccagagggaccagtgccctcctcagtgcttaggggcagagccacctgcagcaatggtatctgcatattagcccctctccaccttctttctcccgctgaatcatttccctcaaagcccaagagctgtcactgcttctttctccctgggaagaatgcgtggactctgcctggtgatagactgaagccagaacagtgccacaccctcgccttaattccttgctaggtgttctcagatttatgagacttcttagtcaaatatgagggaggttggatgtggtggcttgtgcctgtaatcccagcattttgggaagccgaggtgggaggatcccttgaagccaggagtttgagacaagcctgggcaacaaagcaagaccctatctctaaaaaaaaaaaaaaaaaaaaaaaaaaaaaaaaaaaaaaaaaatctaggagatgctctttaccctgcctggcctcaaactattaatagcttcctttgagcaacattatttattatgaactttcaaacacaaaaaagtagagagagtagaataacaaatccccatgagcccatcacccaacttcagtaattatcaattcatggccatcttgttcacccctgcctgcttccctgcttcccctcattctgcagaggttcttttcttttgagacagagtgttgctctgttgcccaggctggagtgcagtggtgcaacttcggctcactgcaacctccgcctcccaggttcaagtgattctcctgcttcagcctctcaagtagctgggattacagatgcccgccaccacacctggctaattttcgtatttttgttagagatggggtttcaccatgttggccaggctggtctcgaactcctgacctcaagtgatccgcccgccttggcctcccaaaatgctgggattacaggtgtgaaccacggtgcctggccactgtacaggttatttatagaagttggagagtgaagggttgagaaagccaaggggcagatgcgggtctggaggattttgtgcctaaggccctctctttgctcccagacagcatgaagtaacaatgaggcatccacctcttggttttgtggcctctgtggatgacgtctctcaccttgaaccagttcagagttggagtagcgcaggatcctgtcttcagaggaggggccgaagcgggttcctctgttgtcaagctctttggaggtgcctggctgctactactgtcccagagaggtgatgatgaatgatgggtgtgtccagtggcagtttgccccactgaggcaggggcttccactaggccctgacagagcccttccagcaggcagaaatccctgtgctaggcaagattcaaactccgtagcatgtctcctgctcccatctcttaggaatggagtccttcaggccttgagtcccacattttccatgatgctccattaagcagctgatagcacccccacctccagggaaagtgagttcagagtccttggtctaatgcatctgtgttgaaattgaggccttcccctgtgttcacctttctgctctttttcttttagcccaaggctatgaaggcctcattcggtgctgggcatggtcactcctagcattcctcactctgttgctaacagcaacagcaataataataagggttacaacttactccataccttactgtctgccaggcattaagctaagtgctttacatatattaagtcatttaatcctcataatgaccctatgaaagagataccatctcaacccaattgacagctggtttgcaagattaggagggatgaaggacccaggggacaatgcgagggaaaactctgaccccggggccccaggctggatgttctttatgcctgtgaaccacagcttatcacatgtctggagttagggaccccacttaaagtgagattttggctggaggtggtggatcatacctataatcccagcactttgggagaccaaggcagaaggactgcttgaggccaggagttcaaaaccagtgtaggtaacagctagaccctatctctacaaaaaatttaaaaattagctgggtgtggtggtatgtgcctcaagttccagctactcaggaggctgaggtgggaggatcacttgagcacaggagtttgaagttacagtgagctatgatggcaccactgcacttcagcctaggcaacagagggagaccctgtctttaaagtacatagaggtttttcacaccaacacatctctgcccagtgtgccaacatctgccacctgctataatagtactataacactcaatatgtaattaatgtagtctcagggatgttatgacaatatgattacaactatcacgtgtgtgcccagccaggctcaatgccccaggctgggcgaggtggggcaggggacacagcctaaaatgccaggcctcaggaagccatttggtttagcagacattgtttattaaaggagttacctatgccagatcgaaggcctaagatgattaagacactatgagtgccttcaagtggttggggacgttcatgattgtggtacagacaaataggctttcacatcattcttttatgtaatcatacaacagatatttgcacctacatgtgcagagcactgtgataggcctcagtgacacagaataatacggcaaagaccccacccgatgagccccctcccaccacccaccagtacagtagggggtggtttaatggagtgttcctggaatatgaagtgggggcaggcattaggggtggcaaagggacaagtgtttatctgatcagttatgtactgtttataataagtaaatcagcagagggggaataatacttagaacctatagagagtaaatctgacaagatgaaatgctgatgaaaatatggaggaaatgaaactctcatgggttttgcagggaatctaagtcagtgctgtgttgtgaatgtaggtgtaccctttgaattcatatgttgaatcctaacccccaaagcaatggcattaagaggtggggcctttggggctgggtatggtggctcatgactgtaatcccagcactttgggatgctggcagggggcagatcacttgaagccaggagtctgagatcagcctggccaacatggtgaaaccccatctgtactaaaaatacaaaaattagccaggtgtgatggcgtacatctgtaatttcagccactcgggaggctgagacaggagaatagcttgaacccagtaggtggagatttcagtgagccgagatcgtgccactgcactccagcctgggtgacagagcgagactccatctcaaaaaaataataaagatgtggggcctgtgggaggtggttaggtcatgagggtggagatcatgaatggggttagcaccttataaaacaggcttgagggagcccttctgtcccttctaccatgtgtggatgcagtgagaaggcaccgtatctctgaagcagagagcccgccctggacactggatctgctggcaccttgatcttggacttcccagcctctagaactgtgagaaataattttttgttgtttacaaattacccaggctaaggtgtttcattgtaacctgaatggaccaagctggtgtgaccctgttggaaaactggcagtatctaccaaaagccgaacatacgtataaactgatccagcagttccactcctgggtatgtacaccacagaaagctatgtccaccgagacattggcaagaatgtttctaaccacacgctgactgtagccccaaacctgaaacaacccaaatgtccatccaccaacccaaatgtccatccacagttgaagctacagtgaagtcacagggtcgaatactactgcacagcaacgaatatgaatgaaaatatcgctatgcacagcaacatggataaatttcacagacatgaggtcaagcaaaagaggtcagagtcctcatcatcaagagagaattcattgtatgattctcttcctacaaaaagtacagaaataagcaaaactgatccatggtgttagaagccaggggaacagttaacaggggagggatactggggaggggcatcctggagtgctggtctacctcatctgggtgttgatttcacgagtattgtcagtttgtttccagactccctgttggagatgtggaaataaaaaccacctaaacaagagcagagaggccatttggtcaaagtttgcaaaggagtcagccatgattgcttgtatttggcaggggtcaaaggcaggcagggactgtgaaatgttatagtggaaaaaaagggaaggctctgggtgtgctgtgattggagattgttggcatggggacagagcggactaactggaggggcatctttggttggttgggggggtatatttggctttctctggttggtctggagttggaagagggggtgtggtggctggggattgggaagaagctggcagccactaagttcagactgttctgggtccgattgctgctgaggctgtggtttggcttccttggcttcccaggctggtcatgggtttctggccagagtctattgtcatatgtggcctggccattgtccagttgtatgttcagtctcttggaaggaagggtattgactctgagaggggccaccatcgctggaatgggggacacacagtacttcctccagctgcctacacccccctagggtcagtggcgcctgcctgtgagggtgagcccaatggctagagggctctgctccaagtcattgcttactacacccacaaacattcttcgttctttaaggcctaacttaaagcccagatcctacaggaaaccttgattagacccctctctttattaagcttcctaagatcaaaccctgcttttgtgtaaatgctgacctccttgcctacattttaaaaacctagagctgggcatgatggccccagcctgtaatcccagtgattcaggagactgaggtgggaggattgctagaagccaggagttcgagaccagcctgggtaacatagctagaccacatctcttaaaataaaatagttaatttagccaggcatgatgatatatgcctgtagtcccaactacttggaaggctgaggtgtgaggatctttgagcccgggaggtcgaggctacagtaagctatgatctcaccactgtactccagcctgggtgacagagcgagacccagactcaaaaaataaaaataaaaaccctgaatatcttccttctacttcttcagtgctgtttttatttaaaaaaaaaaaaaaccagccaaaaccacaactttttactgaagtgtaatgtaaatgctgtaaaaggcagtgaaaggcacaagggaggtggaggggtaggaagggtggaagtggcgggaggaagtggcagggcaggcaaaatgaagggaagccctgggttcttgtcctgcatccgcagccagctcccactttcctcaccctccaggacctgtaaactgtgaggctggaccagttatgtcaaatctgtcctcccccagagctcagtccctctgcccttgggtgtccttggcacaaggcaggctaggctgcaccagcttcctccatctccgtcctgcctcccccatccccaggtgccattcccacaccatctgaatcactgatttcctcgcaatcagacgctatcttccagttaatcacttcgcttgtatttaacataagaaagaaaaaccctttcattatcacatacagctggaaatcggcttcttgcaggaggcgtatccaaaggaattggagaagagataaactggtaattggtgaaagaattactttaattttttttcctacttgctgtcatgatgatgtccttagaattgtgagcccgtggacacttctgtacaataaatctgctattattacttctagaactaca

SEQ ID NO: 6 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 7 - DNA, 2223 bp, Homo sapiens
  source 1..2223: mol_type = genomic DNA
tataaaaatagctcttgttaccggaaataactgttcatttttcactcctccctcctaggtcacacttttcagaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggctgcactcagagaagctgcccttggctgctcgtagcgccgggccttctctcctcgtcatcatccagagcagccagtgtccgggaggcagaagatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagacccagggtcaccaggccagagcctccagtggtctccaagcctctggactgggggctctcttcagtggctgaatgtccagcagagctatttccttccacagggggccttgcagggaagggtccaggacttgacatcttaagatgcgtcttgtccccttgggccagtcatttcccctctctgagcctcggtgtcttcaacctgtgaaatgggatcataatcactgccttacctccctcacggttgttgtgaggactgagtgtgtggaagtttttcataaactttggatgctagtgtacttagggggtgtgccaggtgtctttcatggggccttccagacccactccccacccttctccccttcctttgcccggggacgccgaactctctcaatggtatcaacaggctccttcgccctctggctcctggtcatgttccattattggggagccccagcagaagaatggagaggaggaggaggctgagtttggggtattgaatcccccggctcccaccctgcagcatcaaggttgctatggactctcctgccgggcaactcttgcgtaatcatgactatctctaggattctggcaccacttccttccctggccccttaagcctagctgtgtatcggcacccccaccccactagagtactccctctcacttgcggtttccttatactccacccctttctcaacggtccttttttaaagcacatctcagattacccaaaaaaaaaaaaaaaaaa

SEQ ID NO: 8 - protein, 712 aa, Homo sapiens
MACTGPSLPSAFDILGAAGQDKLLYLKHKLKTPRPGCQGQDLLHAMVLLKLGQETEARISLEALKADAVARLVARQWAGVDSTEDPEEPPDVSWAVARLYHLLAEEKLCPASLRDVAYQEAVRTLSSRDDHRLGELQDEARNRCGWDIAGDPGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQDTRALREQSQHLDGERMQAAALNAAYSAYLQSYLSYQAQMEQLQVAFGSHMSFGTGAPYGARMPFGGQVPLGAPPPFPTWPGCPQPPPLHAWQAGTPPPPSPQPAAFPQSLPFPQSPAFPTASPAPPQSPGLQPLIIHHAQMVQLGLNNHMWNQRGSQAPEDKTQEAE

SEQ ID NO: 9 - DNA, 2727 bp, Homo sapiens
  source 1..2727: mol_type = genomic DNA
ttcccagggcgcgggccgcggggtggagccagcgccctcagcgcgctacggtccgcgggcaactccgcagaagccccagcccccaggaccccaggacccagtggcgcagccggcagccccggatccctgatctgcttgggcagctcctgcagaacctggaacagtgaatgggtaggggacactgggcgtgcagaaggcggggggcagtgtggaacatgccttcaccacctccagcttctgctgccggaggctgcacccacctgtgcccatggcctgcacaggcccatcacttcctagcgccttcgacattctaggtgcagcaggccaggacaagctcttgtatctgaagcacaaactgaagaccccacgcccaggctgccaggggcaggacctcctgcatgccatggttctcctgaagctgggccaggaaactgaggccaggatctctctagaggcattgaaggccgatgcggtggcccggctggtggcccgccagtgggctggcgtggacagcaccgaggacccagaggagcccccagatgtgtcctgggctgtggcccgcttgtaccacctgctggctgaggagaagctgtgccccgcctcgctgcgggacgtggcctaccaggaagccgtccgcaccctcagctccagggacgaccaccggctgggggaacttcaggatgaggcccgaaaccggtgtgggtgggacattgctggggatccagggagcatccggacgctccagtccaatctgggctgcctcccaccatcctcggctttgccctctgggaccaggagcctcccacgccccattgacggtgtttcggactggagccaagggtgctccctgcgatccactggcagccctgcctccctggccagcaacttggaaatcagccagtcccctaccatgcccttcctcagcctgcaccgcagcccacatgggcccagcaagctctgtgacgacccccaggccagcttggtgcccgagcctgtccccggtggctgccaggagcctgaggagatgagctggccgccatcgggggagattgccagcccaccagagctgccaagcagcccacctcctgggcttcccgaagtggccccagatgcaacctccactggcctccctgatacccccgcagctccagaaaccagcaccaactacccagtggagtgcaccgaggggtctgcaggcccccagtctctccccttgcctattctggagccggtcaaaaacccctgctctgtcaaagaccagacgccactccaactttctgtagaagataccacctctccaaataccaagccgtgcccacctactcccaccaccccagaaacatcccctcctcctcctcctcctcctccttcatctactccttgttcagctcacctgaccccctcctccctgttcccttcctccctggaatcatcatcggaacagaaattctataactttgtgatcctccacgccagggcagacgaacacatcgccctgcgggttcgggagaagctggaggcccttggcgtgcccgacggggccaccttctgcgaggatttccaggtgccggggcgcggggagctgagctgcctgcaggacgccatagaccactcagctttcatcatcctacttctcacctccaacttcgactgtcgcctgagcctgcaccaggtgaaccaagccatgatgagcaacctcacgcgacaggggtcgccagactgtgtcatccccttcctgcccctggagagctccccggcccagctcagctccgacacggccagcctgctctccgggctggtgcggctggacgaacactcccagatcttcgccaggaaggtggccaacaccttcaagccccacaggcttcaggcccgaaaggccatgtggaggaaggaacaggacacccgagccctgcgggaacagagccaacacctggacggtgagcggatgcaggcggcggcactgaacgcagcctactcagcctacctccagagctacttgtcctaccaggcacagatggagcagctccaggtggcttttgggagccacatgtcatttgggactggggcgccctatggggctcgaatgccctttgggggccaggtgcccctgggagccccgccaccctttcccacttggccggggtgcccgcagccgccacccctgcacgcatggcaggctggcacccccccaccgccctccccacagccagcagcctttccacagtcactgcccttcccgcagtccccagccttccctacggcctcacccgcaccccctcagagcccagggctgcaacccctcattatccaccacgcacagatggtacagctggggctgaacaaccacatgtggaaccagagagggtcccaggcgcccgaggacaagacgcaggaggcagaatgaccgcgtgtccttgcctgaccacctggggaacacccctggacccaggcatcggccaggaccccatagagcaccccggtctgccctgtgccctgtggacagtggaagatgaggtcatctgccactttcaggacattgtccgggagcccttcatttaggacaaaacgggcgcgatgatgccctggctttcagggtggtcagaactggatacggtgtttacaattccaatctctctatttctgggtgaagggtcttggtggtgggggtattgctacggtcttttaattataataaatatttattgaatgcttccgcagcaaaaa

SEQ ID NO: 10 - protein, 6 aa, Synthetic construct
HHHHHH

(19) United States
(12) Patent Application Publication
(10) Pub. No.: US 2024/0158453 A1
(43) Pub. Date: May 16, 2024

(54) Nucleic Acids That Manipulate Immune Pathways

(71) Applicant: Children`s Medical Center Corporation, Boston, MA (US)

(72) Inventors: Jonathan C. Kagan, Brookline, MA (US); Yunhao Tan, Boston, MA (US)

(21) Appl. No.: 18/214,333

(22) Filed: Jun. 26, 2023

Related U.S. Application Data
(63) Continuation of application No. 16/612,713, filed on Nov. 11, 2019, now abandoned.
(60) Provisional application No. 62/505,819, filed on May 12, 2017.

(51) Int. Cl. C07K 14/47; A61P 35/00; C07K 14/725

(52) U.S. Cl. CPC C07K 14/4702; A61P 35/00; C07K 14/7051; C07K 2319/03; C07K 2319/43; C07K 2319/71

(57) ABSTRACT

The present invention provides methods and compositions of synthetic novel genes to manipulate signaling pathways of the immune system.

### CROSS-REFERENCE TO RELATED APPLICATIONS

This application is a continuation application of U.S. application Ser. No. 16/612,713, filed on Nov. 11, 2019, which is a national stage application filed under 35 U.S.C. § 371, of International Patent Application No PCT/US2018/032479, filed on May 12, 2018, which claims the benefit of and priority to U.S. Provisional Application No. 62/505,819, filed on May 12, 2017, the entire contents of which are incorporated herein by reference in their entirety.

### SEQUENCE LISTING

This application contains a Sequence Listing that has been submitted electronically as an XML file named 37314-0101002_SL_ST26.xml. The XML file, created on Jun. 26, 2023, is 47,020 bytes in size. The material in the XML file is hereby incorporated by reference in its entirety.

### FIELD OF THE INVENTION

The invention relates generally to the fields of synthetic biology for antitumor immunity in cancer.

### BACKGROUND OF THE INVENTION

Work over the last several years has established that anti-tumor immunity does occur naturally, and that tumors that are more immunogenic are better treated by current T-cell based immune-therapies. In particular, the efficacy of several cancer therapies is highly correlated with the expression of interferon genes in the tumor. Current approaches to increase tumor immunogenicity have focused mainly on the injection into the tumor of cytokines or microbial activators of inflammation. Accordingly, there is a need for new therapies to induce antitumor immunity.

### SUMMARY OF THE INVENTION

The invention is based, at least in part, upon the discovery that synthetic novel genes can rewire the signaling pathways of the immune system. It was specifically demonstrated that, a synthetic gene to alter the Toll-like Receptor (TLR) and Interleukin-1 Receptor (IL-1R) signaling pathway induced the expression of interferon-family cytokines. These findings indicated that synthetic genes are capable of inducing a strong interfer on-based antitumor response.

In embodiments, the invention is based on the identification of modified nucleic acid sequences, wherein the modified nucleic acid sequence encodes for a polypeptide, and wherein the polypeptide comprises 1) a sequence of a motif from a signaling or targeting protein which stimulates a response, and 2) a sequence of a peptide that does not induce the response.

In some embodiments, the modified nucleic acid sequence described herein is a synthetic gene. In aspects, the sequence of a motif from a signaling or targeting protein which stimulates a response is appended to the N- or C-terminus of the sequence of a peptide that does not induce the response.

In alternative embodiments, the sequence of a motif from a signaling or targeting protein which stimulates a response is inserted into the sequence of a peptide that does not induce the response.

In particular embodiments, the signaling or targeting protein which stimulates a response is an adaptor protein (e.g., a mitochondrial antiviral-signaling (MAVS), a stimulator of interferon genes (STING), or a TIR-domain containing adaptor-inducing interferon-β (TRIF)). In certain embodiments, the signaling or targeting protein which stimulates a response is an adaptor protein comprises a polypeptide motif having a 50% sequence identity to SEQ ID NO: 1: VTMNAPMTSVAPPPSVLSQEPRLLISGMDQPLPLRTDLI, or fragment thereof. In certain embodiments, the signaling or targeting protein which stimulates a response is an adaptor protein comprises a polypeptide motif having a 70% sequence identity to SEQ ID NO: 1: VTMNAPMTSVAPPPSVLSQEPRLLISGMDQPLPLRTDLI, or fragment thereof. In certain embodiments, the signaling or targeting protein which stimulates a response is an adaptor protein comprises a polypeptide motif having an 80% sequence identity to SEQ ID NO: 1: VTMNAPMTSVAPPPSVLSQEPRLLISGMDQPLPLRTDLI, or fragment thereof. In certain embodiments, the signaling or targeting protein which stimulates a response is an adaptor protein comprises a polypeptide motif having a 90% sequence identity to SEQ ID NO: 1: VTMNAPMTSVAPPPSVLSQEPRLLISGMDQPLPLRTDLI, or fragment thereof. In certain embodiments, the signaling or targeting protein which stimulates a response is an adaptor protein comprises a polypeptide motif having a 91%, 92%, 93%, 94%, 95%, 96%, 97%, 98%, 99%, or 99.9% sequence identity to SEQ ID NO: 1: VTMNAPMTSVAPPPSVLSQEPRLLISGMDQPLPLRTDLI, or fragment thereof. In certain embodiments, the signaling or targeting protein which stimulates a response is an adaptor protein comprises a polypeptide motif comprising SEQ ID NO: 1: VTMNAPMTSVAPPPSVLSQEPRLLISGMDQPLPLRTDLI, or fragment thereof.

In further embodiments, the protein that does not induce the response may include, but is not limited to, myeloid differentiation primary response gene 88 (MyD88), Asc, RHIM, RIPK3, and Casp8CAT.

In some aspects, the disclosure provided herein describes a composition for eliciting antitumor immunity in a cancer, the composition comprising a synthetic gene, wherein the synthetic gene comprises a motif encoded from a signaling or targeting protein which stimulates a response and is appended to a protein that does not induce the response.

In some aspects, the signaling or targeting protein which stimulates a response is an adaptor protein.

In exemplary aspects, the adaptor protein is selected from, but not limited to a mitochondrial antiviral-signaling (MAVS), stimulator of interferon genes (STING), and TIR-domain containing adaptor-inducing interferon-β (TRIF).

In certain embodiments, a chimeric nucleic acid sequence comprises a myeloid differentiation primary response gene 88 (MyD88) sequence and one or more sequences comprising mitochondrial antiviral-signaling (MAVS), stimulator of interferon genes (STING), TIR-domain containing adaptor-inducing interferon-β (TRIF), fragments or combinations thereof. In certain embodiments a nucleic acid a sequence encoded by a mitochondrial antiviral-signaling (MAVS), stimulator of interferon genes (STING), TIR-domain containing adaptor-inducing interferon-β (TRIF), encode for a peptide comprising a hydrophilic residue; at least one amino acid residue and a phosphorylation site.

In certain embodiments, a method of reprogramming a signaling organelle, comprising contacting a cell with the modified nucleic acid sequence or a chimeric nucleic acid sequence of embodied herein.

In some aspects, the motif comprises about 1-50% of the modified nucleic acid sequence encoding a polypeptide. In further aspects, the motif comprises about 1-40%, about 1-3-30%, about 1-20%, about 1-10%, about 1-5% or about 1-2%. Alternatively, the motif can comprise about 5-50% of the modified nucleic acid sequence encoding a polypeptide, alternatively, about 5-40%, about 5-30%, about 5-20%, about 5-10%. Alternatively, the motif can comprise about 10-50% of the modified nucleic acid sequence encoding a polypeptide, alternatively about 10-40%, about 20-40%, about 30-40%. In exemplary embodiments, the motif can comprise about 10% of the modified nucleic acid sequence encoding a polypeptide.

In aspects, the motif comprises a hydrophilic or hydrophobic motif, and in certain embodiments, the motif may be a pLxIS motif.

In embodiments, the adaptor protein comprises a polypeptide motif comprising of SEQ ID NO: 1: VTMNAPMTSVAPPPSVLSQEPRLLISGMDQPLPLRTDLI, or fragment thereof.

In embodiments, the protein that does not induce the response is selected from the group consisting of myeloid differentiation primary response gene 88 (MyD88), Asc, RHIM, RIPK3, and Casp8CAT.

In exemplary aspects, the response is an immune response induces the expression of interferon-family cytokines (i.e., phosphorylation), autocrine signals (i.e., from the immune system), signals in the inflammatory pathway (e.g., the inflammasome), metabolic pathways, and the like. In exemplary aspects, the response can induce or suppress cell division, differentiation, and cell-cell communication, and migration, phagocytosis, and the like.

In embodiments, the composition described herein is used to treat cancer, and the cancer is selected from the group consisting of sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer including prostate adenocarcinoma, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, retinoblastoma, multiple myeloma, rectal carcinoma, thyroid cancer, head and neck cancer, brain cancer, cancer of the peripheral nervous system, cancer of the central nervous system, neuroblastoma, colorectal adenocarcinoma and cancer of the endometrium.

Also provided herein are pharmaceutical compositions comprising a composition of described herein. In some examples, the pharmaceutical composition also comprises a pharmaceutically acceptable carrier (i.e., an aqueous carrier or a solid carrier).

In further embodiments, methods for treating a neoplasia in a subject comprising administering the composition described herein are disclosed. In examples, the neoplasia is a cancer, and the cancer is selected from the group consisting of sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer including prostate adenocarcinoma, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, retinoblastoma, multiple myeloma, rectal carcinoma, thyroid cancer, head and neck cancer, brain cancer, cancer of the peripheral nervous system, cancer of the central nervous system, neuroblastoma, colorectal adenocarcinoma and cancer of the endometrium.

Furthermore, provided herein are methods for inducing antitumor immunity in a cancer cell of a subject comprising administering the composition of the invention described herein to the subject (e.g., a human subject).

Other aspects of the invention are described in, or are obvious from, the following disclosure, and are within the ambit of the invention.

### BRIEF DESCRIPTION OF THE DRAWINGS

FIG. 1 is a schematic showing the functional convergence and divergence between innate adaptor proteins mediated TBK1 phosphorylation.

FIG. 2 is a schematic showing mechanisms by which to rewire PRR signaling via synthetic biology approaches.

FIG. 3 is a schematic showing the synthetic modulation of myddosome activity.

FIGS. 4A and 4B are schematics showing the generation of MyD88 alleles containing the pLxIS Motif, either at C- or N-terminus.

FIG. 5 is an image showing that the overexpression of the MyD88-pLxIS alleles induced IRF3 phosphorylation.

FIG. 6A is a schematic showing that MyD88-pLxIS alleles restored TBK1 phosphorylation and induced IRF3 phosphorylation in response to LPS and P3C.

FIG. 6B is a schematic showing that MyD88-pLxIS alleles restored TBK1 phosphorylation and induced IRF3 phosphorylation in response to LPS and P3C.

FIGS. 7A and 7B are bar graphs showing that MyD88-pLxIS alleles rescued Ill-b and viperin expression in response to LPS and P3C.

FIG. 8 is an image showing that the expression levels of MyD88 Alleles were comparable to that of endogenous MyD88 in WT iBMDMs.

FIGS. 9A and 9B are images showing that TBK1 activity is required for IRF3 phosphorylation induced by MyD88-pLxIS alleles.

FIGS. 10A and 10B are schematics showing that phosphorylation of the Ser residue in the pLxIS modify TBK1 is essential for IRF3 activation.

FIG. 11 is an image showing that S365 in the pLxIS Motif is required for IRF3 activation induced by MyD88-pLxIS overexpression.

FIG. 12A is an image showing that S365 in the pLxIS motif is essential for IRF3 activation.

FIG. 12B is an image showing that S365 in the pLxIS motif is essential for IRF3 activation.

FIG. 12C is a schematic showing that S365 in the pLxIS motif is essential for IRF3 activation.

FIG. 13A is an image showing that a S365 in the pLxIS Motif is essential for IRF3 activation.

FIG. 13B is an image showing that a S365 in the pLxIS motif is essential for IRF3 activation.

FIG. 13C is a schematic showing that a S365 in the pLxIS motif is essential for IRF3 activation.

FIG. 14A is a schematic showing the pLxIS Motif is essential for IRF3 activation.

FIGS. 14B-14C are bar graphs showing that S365 in the pLxIS motif is essential for IRF3 activation.

FIGS. 15A and 15B are bar graphs showing that MyD88 oligomerization/myddosome formation is required for IFN responses.

FIG. 16 is a schematic depicting that the myddosome can be rewired to trigger distinct forms of cell death.

FIG. 17 is an image showing that the expression levels of distinct MyD88-death alleles in MyD88×TRIF DKO iBMDMs.

FIG. 18 is a bar graph showing that MyD88-RIPK3 allele in MyD88×TRIF DKO iBMDMs lead to IL-10 release upon LPS and P3C stimulation.

FIG. 19 is an image showing that adaptor proteins of SMOCs are versatile platforms for rewiring signaling circuits.

FIGS. 20A and 20B are images showing a diverse cell types form Myddosome upon TLR activation.

FIG. 21A is an image showing that diverse cell types form Myddosome upon TLR activation.

FIG. 21B is an image showing that diverse cell types form Myddosome upon TLR activation.

FIG. 22 is a schematic showing host responses controlled by PRR signaling Fig is an image showing the Toll-like Receptor (TLR) family, a genetically well-defined PRR family.

FIG. 24 is an image showing the Toll-like Receptor (TLR) family, a genetically well-defined PRR family. Owing to the technical advances in genetic sequencing, proteomic, and transcriptomic, hundreds of host proteins in the TLR pathway are known, and knowledge of the host transcriptional responses has been refined to a single-cell level. This progress to the foundation to study the cell biological and biochemical functions of the host proteins in the pathway.

FIG. 25 is an image showing how TLR signaling operates in space and time during microbial encounter remains unclear. A common challenge in the field is how different host components work in space and time during microbial encounter. Knowledge from this aspect will ultimately allow for the harnessing the power of the innate immune system. For instance, to boost the pathway to fight infectious agents, to fine-tune the pathway for vaccination, or to cut the pathway to treat immunopathology such as sepsis.

FIG. 26 is an image showing that TLR pathway proteins operate in a coordinated manner. Upon activation, TLR4 engages four cytosolic adaptor proteins containing the Toll/IL-1 Receptor Homology domain (TIR).

FIG. 27 is an image showing that different sorting and signaling adaptor pairs diversify host responses at distinct subcellular sites. For example, they could be functionally categorized into sorting adaptors and signaling adaptors, by which sorting adaptors link the activated receptor to signaling adaptors to trigger host response.

FIG. 28 is an image depicting a paradox in TLR-mediated signal transduction. Immediately downstream of receptor trafficking and upstream of transcriptional responses within the nucleus, lies the process that is known as signal transduction.

FIG. 29 is an image showing the Supramolecular Organization Centers SMOCs.

FIG. 30 is an image depicting Supramolecular Organization Centers SMOCs. In particular, from a structural biology perspective, these higher-order helical structures serve as platforms to recruit effector molecules such as kinases to amply and propagate downstream signaling events.

FIG. 31 is a schematic depicting the common features of SMOCs. In particular, each consists of a Receptor-Adaptor-Effector Protein Complex, each is assembled upon ligand/microbe encounters, and there is an incomplete understanding of their natural composition and biological activities.

FIG. 32 is an image depicting that the innate immune system is the sentinel between the host and the microbes

FIG. 33 is an image depicting that the pattern recognition receptors recognize microbe associated molecular patterns.

FIG. 34 is an image depicting that the Toll-like Receptor (TLR) Family is one of the best genetically defined PRR families; for example, a genome-wide CRISPR screen in primary immune cells to dissect regulatory networks.

FIG. 35 is an image depicting that CD14 controls TLR4 endocytosis and MD-2 selects TLR4 as cargo. GPI anchor protein CD14 was identified to activate an endocytosis pathway composed of ITAM adaptors, Syk kinase, and phospholipase Cr2 to bring TLR4 in to the cell. Strikingly, TLR4 signaling was not required for this endocytosis event.

FIGS. 36A and 36B are images showing that TIRAP is the first cellular regulator of the myddosome.

FIG. 37 is an image depicting the Central Hypothesis: The Myddosome Functions as a Signaling Platform to Coordinate Diverse Cellular Processes Upon TLR Activation; in addition to the well-characterized transcriptional responses, TLR pathway activation has also been implicated in diverse host responses such as metabolic reprogramming, autophagy ROS production cell death etc., which are non-transcriptional responses. Whereas myddosome formation is known to activate NF-kB activation, it is largely unclear whether Myddosome formation induces these other responses. Therefore, the Myddosome is a signaling platform that coordinate diverse cellular processes upon TLR activation was hypothesized

FIG. 38 is an image showing that TLR activation induces media acidification in primary and immortalized cells.

FIGS. 39A and 39B are bar graphs depicting that TLR activation promotes glycolysis in primary and immortalized cells.

FIGS. 40A and 40B are a graph and an image depicting that TLR activation promotes rapid glycolytic burst in iBMDMs.

FIGS. 41A-41C are graphs depicting that 2-DG treatment did not affect host responses at the receptor proximal.

FIGS. 42A and 42B are bar graphs depicting that inhibition of glycolysis by 2-DG uncouples cytokine gene transcription from translation.

FIG. 43 is a blot showing how TLR activation induce glycolysis, i.e. that the protein kinase Akt might be critical for early phase TLR-mediated glycolysis.

FIG. 44 is a graph depicting that inhibition of Akt activation dampens TLR-mediated glycolytic burst.

FIG. 45 is a gel depicting that MyD88 signaling primarily drives Akt phosphorylation.

FIG. 46 is a gel depicting that chemical inhibitors targeting TBK1 activity reduce Akt phosphorylation in WT iBMDMs.

FIG. 47 is a gel depicting that chemical inhibitors targeting TBK1 activity reduce Akt

FIG. 48 is a graph depicting that chemical inhibitors of TBK1/IKKe and AKT dampen TLR dependent glycolysis activation.

FIGS. 49A and 49B are bar graphs depicting that TBK1 inhibitors do not affect NF-kB activation at the transcriptional level.

FIGS. 50A and 50B are bar graphs depicting that TBK1 is dispensable for pro-inflammatory cytokine gene expression

FIG. 51 is a bar graph depicting that pro-inflammatory cytokine production is inhibited by chemical inhibitors of TBK1/IKKe.

FIG. 52 is an image depicting art regarding which host factor(s) promote TBK1 activation.

FIG. 53 is a gel depicting that TLR signaling promotes TBK1 phosphorylation independent of TRIF-IRF3 signaling axis.

FIG. 54 is a gel depicting that MyD88 signaling promotes efficient TBK1 phosphorylation in the TLR4 pathway.

FIG. 55 is a gel depicting that MyD88 signaling promotes efficient TBK1 phosphorylation in the TLR2 pathway.

FIG. 56 is a gel depicting that TBK1 is associated with the Myddosome in responses to surface TLR ligands.

FIGS. 57A and 57B are gels depicting that TBK1 is a novel component of the myddosome.

FIGS. 58A and 58B are images depicting that the Canonical components of the myddosome interact with each other via homotypic interactions.

FIG. 59 is a gel depicting that myddosome formation in living cells are regulated by distinct post-translational modifications.

FIG. 60 is a gel depicting that components of the myddosome are phosphorylated

FIG. 61 is an image depicting whether major components of the myddosome subjected to ubiquitination; i.e., halo-Tab2 pulldown: an Affinity purification strategy to isolate K63-ubiquitinylated proteins.

FIGS. 62A and 62B are images depicting that myddosome components are associated with ubiquitin chains (K63).

FIG. 63 is an image depicting that in living cells post-translational modifications create platforms for protein-protein interactions.

FIG. 64 is a gel indicating that TRAF6 might regulate TBK1 phosphorylation.

FIG. 65 is an image depicting distinct biological roles of TBK1 in TLR signaling.

FIG. 66 is a blot indicating the validation of the observations from chemical perturbation of TBK1 function with genetics.

FIGS. 67A and 67B are gels depicting that TBK1 is activated upon microbial encounters.

FIGS. 68A and 68B are gels depicting that TBK1 is activated upon microbial encounters.

FIGS. 69A-69I show that TLR activation induces myddosome formation and TBK1-mediated early glycolysis in macrophages. FIG. 69A: WT and Tlr4^{-/-} iBMDMs were lysed and TLR4 isolated (left) via immunoprecipitation. WT iBMDMs (middle) and RAW264.7 cells expressing TLR9-HA (right) were treated for the indicated times with LPS (100 ng/ml) or CpG (5 μM), respectively. Cells were lysed. TLR4 and TLR9 were isolated from cell lysates via immunoprecipitation. TLRs, IRAK2, IRAK4, and MyD88 were detected by western analysis. Actin was probed as loading control. Time point “0” indicates controls. FIG. 69B: Primary BMDMs (left) and iBMDMs (right) were stimulated with LPS (100 ng/ml), P3C (1 μg/ml), and R848 (1 μg/ml) for the indicated time points and lysed. MyD88 was immunoprecipitated from cell lysates and IRAK2, IRAK4, and TRAF6 were detected by western blot. FIG. 69C: iBMDMs expressing 3×FLAG-TRAF6 were stimulated with LPS (100 ng/ml), P3C (1 μg/ml), and R848 (1 μg/ml) for the indicated time points and lysed. 3×FLAG-TRAF6 was immunoprecipitated from cell lysates via an anti-FLAG antibody (M2). IRAK2, IRAK4, and MyD88 were detected by western blot. FIG. 69D: Primary BMDMs were seeded in a Seahorse XF-96 analyzer. TLR induced real-time changes in the ECAR of primary BMDMs stimulated with LPS (100 ng/ml), P3C (1 μg/ml), and R848 (1 μg/ml) with or without 2-DG (25 mM) or left untreated (NT) were measured by the Seahorse assay. The readout of ECAR is presented as relative fold change in comparison to the basal levels before inhibitor incubation, which is set to 1 by the Seahorse analyzer. Data represent mean±SEM of triplicate wells. Shown is one representative experiment out of three independent experiments. FIG. 69E: TLR induced real-time changes in the ECAR of primary BMDMs stimulated with LPS (100 ng/ml), P3C (1 μg/ml), and R848 (1 μg/ml) with or without Actinomycin (ActD, 1.5 μg/ml) or left untreated (NT) were measured by the Seahorse assay. The readout of ECAR is presented as relative fold change in comparison to the basal levels before inhibitor incubation, which is set to 1 by the Seahorse analyzer. Data represent mean±SEM of triplicate wells. Shown is one representative experiment out of three independent experiments. FIG. 69F: Cell lysates from indicated iBMDM lines were separated by SDS-PAGE. Expression levels of endogenous TBK1 and IKKF were determined by western analysis. FIG. 69G: Indicated iBMDM lines were stimulated with LPS (100 ng/ml) for 4 hours. The expression of Rsad2 was measured by qPCR. FIG. 69H: Indicated iBMDMs were stimulated with LPS (100 ng/ml), P3C (1 μg/ml), and R848 (1 μg/ml) or left untreated (NT). Real-time changes in the ECAR were measured by the Seahorse assay. The readout of ECAR is presented in the form of relative fold change in comparison to the basal levels before TLR activation, which is set to 1 by the Seahorse analyzer. Data represent mean±SEM of triplicate wells. Shown is one representative experiment out of three independent experiments. FIG. 69I: Indicated iBMDM lines were pre-treated with cycloheximide (50 μg/ml) then treated with LPS (100 ng/ml) for the times indicated and lysed. Western analysis was used to monitor the early phase activation of NF-κB (pp65, IκBα) and MAP kinase (pp38, pERK) pathways. Actin was probed as a loading control. For western analysis, representative blots from at least three independent experiments were shown.

FIGS. 70A-70H show that the myddosome is the primary driver of TBK1 activation and glycolytic metabolism during TLR signal transduction. FIG. 70A: Primary BMDMs with indicated genotypes were stimulated with TLR ligands (or not). Real-time alterations in the ECAR were monitored by the Seahorse analyzer. The readout of ECAR is shown as relative fold change in comparison to the basal levels before TLR stimulation, which is normalized to 1 by the Seahorse analyzer. Data represent mean±SEM of triplicate wells. Shown is one representative experiment out of three independent experiments. FIG. 70B: Myd88^{-/-}/Trif^{-/-} iBMDMs retrovirally expressing MyD88 or an empty vector (VT) were stimulated with indicated TLR ligands (or not). Real-time changes in the ECAR were monitored by the Seahorse analyzer. The readout of ECAR is shown as relative fold change in comparison to the basal levels before TLR stimulation, which is normalized to 1 by the Seahorse analyzer. Data represent mean±SEM of triplicate wells. Shown is one representative experiment out of three independent experiments. FIG. 70C: Primary BMDMs stimulated with TLR ligands for the designated time points were lysed. pTBK1, TBK1, pIRF3, and IRF3 were detected by western analysis. FIG. 70D: Primary BMDMs with indicated genotypes were LPS (100 ng/ml) for the indicated times. Cells were lysed. pIRF3 and IRF3 were detected by western blot. FIG. 70E: Primary BMDMs with indicated genotypes were stimulated with TLR ligands for the indicated times. Cells were lysed. pTBK1, TBK1, and MyD88 were detected by western blot. FIG. 70F: Myd88^{-/-}/Trif^{-/-} iBMDMs retrovirally expressing MyD88 or an empty vector (VT) were stimulated with TLR ligands for the times indicated. Cells were lysed. pTBK1, TBK1, and MyD88 were detected by western blot. FIG. 70G: Primary BMDMs with indicated genotypes were stimulated with TLR ligands for the indicated time points. pAKT and AKT were detected by western blot. FIG. 70H: Myd88^{-/-}/Trif^{-/-} iBMDMs retrovirally expressing MyD88 or an empty vector (VT) were stimulated with indicated TLR ligands for the times indicated. Cells were lysed. pAKT1 and AKT were detected by western blot. For western analysis, representative blots from at least three independent experiments were shown.

FIGS. 71A-71I show that TBK1 is a novel component of the myddosome. FIG. 71A: iBMDMs were stimulated with TLR ligands for times indicated. Cells were lysed and MyD88 was immunoprecipitated from the lysates. pTBK1, TBK1, and MyD88 were determined by western analysis. FIG. 71B: 3×FLAG-MyD88-expressing Myd88^{-/-}/Trif^{-/-} iBMDMs were stimulated with TLR ligands for times indicated. Myddosomes were isolated using the M2 anti-FLAG antibody. Components of the myddosome were determined by western analysis. FIG. 71C: Myd88^{-/-}/Trf^{-/-} iBMDMs expressing 3×FLAG-MyD88-GyrB were stimulated with TLR ligands for times indicated. Components of the myddosome were determined by western analysis. FIG. 71D: Myd88^{-/-}/Trf^{-/-} iBMDMs expressing 3×FLAG-MyD88-GyrB were stimulated with Coumermycin (CM) (0.5 μM), LPS (100 ng/ml), and P3C (1 μg/ml) for 4 hr. mRNA was extracted. Il-1b transcripts were analyzed by qPCR. FIGS. 71E and 71F: Myd88^{-/-}/Trif^{-/-} iBMDMs expressing 3×FLAG-MyD88-GyrB were stimulated with CM (0.5 μM) for 30 min and fixed. Cells were stained with antibodies detecting FLAG (for MyD88) and pTBK1. Cytosol was visualized via the expression of the IRES-GFP from the retroviral vector and was pseudo-colored in blue (FIG. 71E). Quantification of the colocalization between pTBK1 and MyD88 staining (FIG. 71F). Images are representative of at least three independent experiments where more than 100 cells were examined per condition. The scale bar represents 5 μm. FIG. 71G: RAW264.7 cells expressing shTRAF6 and shCTRL were stimulated with TLR ligands for 15 min and lysed. pTBK1, TBK1, TRAF6, and Actin were detected by western analysis. FIG. 71H: RAW264.7 cells expressing shTRAF6 and shCTRL were stimulated with TLR ligands for 4 h. mRNA was extracted. Il-1b and Il-6 transcripts were analyzed by qPCR. FIG. 71I: RAW264.7 cells expressing shTRAF6 and shCTRL were stimulated with TLR ligands for times indicated. MyD88 was immunoprecipitated and myddosome components were determined by western analysis. For western analysis, representative blots from at least three independent experiments were shown.

FIGS. 72A-72H show that synthetic myddosomes induce type I IFN responses upon TLR stimulation. FIG. 72A: Schematic representation of the MyD88-pLxIS alleles. FIG. 72B: Myd88^{-/-}/Trif^{-/-} iBMDMs expressing MyD88, MyD88-NpLxIS, and MyD88-CpLxIS were stimulated with TLR ligands [LPS (1 μg/ml), P3C (1 μg/ml), and R848 (1 μg/ml)] for 90 min and lysed. Expression of different MyD88 alleles and activation the type-I IFN (pSTAT1/STAT1 and pIRF3/IRF3) pathway were examined by western blot. FIG. 72C: Myd8^{-/-}/Trif^{-/-} iBMDMs expressing MyD88, MyD88-NpLxIS, and MyD88-CpLxIS were stimulated with TLR ligands for 4 hr. mRNA was extracted, Il-1b and Rsad2 transcripts were determined by qPCR. FIG. 72D: Myd88^{-/-}/Trif^{-/-} iBMDMs expressing MyD88, MyD88-NpLxIS, and MyD88-CpLxIS were stimulated with TLR ligands for 6 hr. Secreted TNFα and IFNβ were measured by ELISA. FIG. 72E: Schematic representation of the selected MyD88-CpLxIS mutant alleles. FIG. 72F: Myd88^{-/-}/Trif^{-/-} iBMDMs expressing MyD88-CpLxIS and its mutant alleles were treated with TLR ligands for 90 min and lysed. Expression of different MyD88 alleles and activation the type-I IFN (pSTAT1/STAT1 and pIRF3/IRF3) pathway were examined by western blot. FIG. 72G: Myd88^{-/-}/Trif^{-/-} iBMDMs expressing MyD88-CpLxIS and its mutant alleles were treated with TLR ligands for 4 hr. mRNA was extracted. Il-1b and Rsad2 transcripts were determined by qPCR. Data represent mean±SEM of triplicate wells. Shown is one representative experiment out of three independent experiments. FIG. 72H: Myd88^{-/-}/Trif^{-/-} iBMDMs expressing MyD88-CpLxIS and its mutant alleles were treated with TLR ligands for 4 h. Secreted TNFα and IFNβ were measured by ELISA. Data represent mean±SEM of triplicate wells. Shown is one representative experiment out of three independent experiments. For western analysis, representative blots from at least three independent experiments were shown.

FIGS. 73A-73G show that a synthetic myddosome promotes RIP3-dependent necroptosis upon TLR stimulation. FIG. 73A: Schematic representation of the MyD88-RIP3 allele (FIG. 73B and FIG. 73C) Myd8^{-/-}/Trif^{-/-} iBMDMs expressing MyD88, MyD88-RIP3, and an empty vector (VT) were stimulated with TLR ligands [LPS (1 μg/ml); P3C (1 μg/ml), and R848 (1 μg/ml)] for times indicated. Membrane rupture was determined by PI staining (5 μM) (FIG. 73B) and extracellular LDH in the culture media was quantified (FIG. 73C). Data represent mean±SEM of triplicate wells of three independent experiments. FIG. 73D: Myd88^{-/-}/Trif^{-/-} iBMDMs expressing MyD88 and MyD88-RIP3 were analyzed by confocal microscopy. Cells were stimulated with P3C (1 μg/ml) in PI-containing (5 μM) medium. One image was captured every 3 min for around 60 min. Shown are representative frames from a capture. The scale bar represents 10 μm. FIGS. 73E and 73F: Myd88^{-/-}/Trif^{-/-} iBMDMs expressing MyD88-RIP3 were pre-treated (or not) with GSK872 (2.5 μM) were stimulated with TLR ligands for indicated times. Membrane rupture was determined by PI staining (5 μM) (FIG. 73E) and extracellular LDH in the culture media was quantified (FIG. 73F). Data represent mean±SEM of triplicate wells of three independent experiments. FIG. 73G: Cells were treated as described in (FIGS. 73E and 73F). Images of cell morphology were taken 1 h post-stimulation. The arrow head highlights a dead cell. The scale bar represents 10 μm. Images are representative of at least three independent experiments. For western analysis, representative blots from at least three independent experiments were shown.

FIG. 74A are western blots showing that the recruitment of TRAF6 was specific, as it could be only detected in MyD88 immunoprecipitates from LPS stimulated cells, but not in the IgG control immunoprecipitates. iBMDMs were stimulated with LPS (100 ng/ml) for the times indicated and lysed. Cell lysates were incubated with an MyD88-specific antibody or control IgG. Western analysis was used to detect TAK1, NEMO, and IKKβ (left); IRAK2, IRAK4, TRAF6, and MyD88 (Right). FIG. 74B is a western blot showing that the interacdions of MyD88 and IRAK kinases with 3×FLAG-TRAF6 were specific. 3×FLAG-TRAF6 expressing iBMDMs were stimulated with LPS (100 ng/ml) for the times indicated and lysed. Cell lyates were incubated with an anti-FLAG antibody (M2) or control IgG. Western blot was used to detect IRAK2, IRAK4, MyD88, and TRAF6. Actin was probed as a loading control. FIGS. 74C and 74D: Real-time responses in the ECAR and OCR from primary BMDMs (FIG. 74C) and iBMDMs (FIG. 74D) treated (or not) with indicated TLR ligands were measured by the Seahorse assay. Shown are the un-normalized data from the Seahorse analyzer. Data represent mean±SEM of triplicate wells. Shown is one representative experiment out of three independent experiments. FIG. 74E: Primary BMDMs were pretreated (or not) with Actinomycin D (ActD, 1.5 μg/ml) or not for 40 min, followed by stimulation with (or not) indicated TLR ligands for an additional 4 h. Il-1b and Il-6 transcripts were determined by qPCR. FIG. 74F: TLR induced real-time changes in the ECAR of primary BMDMs stimulated with LPS (100 ng/ml), P3C (1 μg/ml), and R848 (1 μg/ml) with or without TBK1/IKKF inhibitors (Inh. BX795, 5 μM; MRT67307, 2.5 μM) or left untreated (NT) were measured by the Seahorse assay. The readout of ECAR is shown as relative fold change in comparison to the basal levels before inhibitor treatment, which is normalized to 1 by the Seahorse analyzer. Data represent mean±SEM of triplicate wells. Shown is one representative experiment out of three independent experiments. FIG. 74G: Indicated iBMDM lines were stimulated with TLR ligands for indicated times and lysed. pAKT, total AKT, pTBK1, and total TBK1 were detected by western blot. FIG. 74H: TLR induced real-time changes in the ECAR of primary BMDMs stimulated with LPS (100 ng/ml), P3C (1 μg/ml), and R848 (1 μg/ml) with or without an AKT inhibitor (AKTi: Triciribine, 20 μM) or left untreated (NT) were measured by the Seahorse assay. The ECAR data are shown as relative fold change in comparison to the basal levels before inhibitor treatment, which is normalized to 1 by the Seahorse analyzer. Data represent mean±SEM of triplicate wells. Shown is one representative experiment out of three independent experiments. For western analysis, representative blots from at least three independent experiments were shown.

FIG. 75A is a western blot recruitment of TBK1 to the myddosome was specific, as no such recruitment could be detected in IgG immunoprecipitates. 3×FLAG-MyD88-expressing Myd88^{-/-}/Trif^{-/-} iBMDMs were stimulated with LPS for times indicated. Cell lysates were incubated with the M2 anti-FLAG antibody or control IgG. Components of the myddosome were determined by western analysis. FIG. 75B: are images showing that phosphorylated p38 (pp38), which is also activated by MyD88, was not detected within MyD88 specks in CM-stimulated cells. Myd88^{-/-}/Trif^{-/-} iBMDMs expressing 3×FLAG-MyD88-GyrB were stimulated with CM (0.5 μM) for 30 min and fixed. Cells were stained with antibodies specific for FLAG (for MyD88) and pp38. Cytosol was visualized via the expression of the IRES-GFP from the retroviral vector and was-pseudo colored in blue. Images are representative of at least three independent experiments where more than 100 cells were examined per condition. The scale bar represents 5 μm. FIG. 74C: 293T cells were co-transfected with HA-TBK1 and indicated GFP-tagged myddosome components in a pairwise manner. 24 hr after transfection, cells were lysed. TBK1 was isolated via an HA-specific antibody, and the immunoprecipitates were analyzed by western blot. FIG. 75D: 293T cells were transfected with HA-TBK1 and FLAG-TRAF6 following the indicated combinations. 24 hr after transfection, cells were lysed. TRAF6 was isolated via the M2 FLAG antibody, and the immunoprecipitates were analyzed by western blot. For western analysis, representative blots from at least three independent experiments were shown.

FIG. 76A is a blot showing that the introduction of WT MyD88 into Myd88^{-/-}/Triff^{-/-} iBMDMs restored phosphorylation of TBK1 and p65, Il-1b gene expression, and TNFα secretion (FIG. 76A)Myd88^{-/-}/Trif^{-/-} iBMDMs expressing MyD88, MyD88-NpLxIS, and MyD88-CpLxIS were stimulated with TLR ligands for 90 min and lysed. pTBK1, TBK1, pp65, and p65 were examined by western blot. FIG. 76B: Myd88^{-/-}/Triff^{-/-} iBMDMs expressing MyD88-CpLxIS and its mutant alleles were treated with TLR ligands for 90 min and lysed. pTBK1, TBK1, pp65, and p65 were examined by western blot. For western analysis, representative blots from at least three independent experiments were shown.

FIG. 77A are images showing the morphological changes that resulted from TLR-mediated MyD88-RIP3 activation were distinct from those induced by the apoptosis-inducing agent staurosporine. Myd88^{-/-}/Trif^{-/-} iBMDMs expressing MyD88-RIP3 were treated with TLR ligands and staurosporine (STS) (1 μM) for 1 hr. Images of cell morphology were taken 1 hr post-stimulation. The arrow head highlights a dead cell. The scale bar represents 10 μm. Images are representative of at least three independent experiments. FIG. 77B: Myd88^{-/-}/Trif^{-/-} iBMDMs expressing MyD88-RIP3 were treated with TLR ligands and staurosporine (STS) (1 μM) for 6 hr and lysed. PARP and Actin were detected by western analysis. FIGS. 77C and 77D: Myd88^{-/-}/Triff^{-/-} iBMDMs expressing MyD88-RIP3 were treated with TLR ligands (or not) and inhibitors (Nec-1 5 μM; ZVAD 10 μM) (or not). Membrane rupture was determined by PI (5 μM) staining (FIG. 77C) and extracellular LDH in the culture media was quantified (FIG. 77D). Data represent mean±SEM of triplicate wells of three independent experiments. FIG. 77E: Primary BMDMs were stimulated with LPS (or not) in the presence of indicated inhibitors (or not) (ZVAD 10 μM; GSK872 2.5 μM) for 18 hr. LDH release in the culture media was quantified. Data represent mean±SEM of triplicate wells of three independent experiments. For western analysis, representative blots from at least three independent experiments were shown.

### DETAILED DESCRIPTION OF THE INVENTION

The present invention relates, at least in part, to the unexpected observation that synthetic novel genes can be created that are capable of rewiring signaling pathways of the immune system, thereby inducing antitumor immunity. In aspects, the invention provides a synthetic gene that elicits antitumor immunity. In some aspects, the synthetic gene induces an immunogenic transcriptional response (i.e., induction of the expression of interferon-family cytokines).

In some aspects, the composition for manipulating an immune pathway, is described, the composition comprising a synthetic gene, wherein the synthetic gene comprises a motif encoded from a signaling and/or targeting protein which stimulates a response, and is appended to a protein that does not induce the response. In some aspects a composition for eliciting antitumor immunity in a cancer is described, the composition comprising a synthetic gene wherein the synthetic gene comprises a motif encoded from an adaptor protein (e.g., MAVS, STING, TRIF, and the like) is appended to a protein that does not induce the response.

As described herein, the term “appended” can mean any known technique in the art to modify genes. This can include for example, standard cloning and molecular biology techniques. In examples, appended can refer to a modification at either the N- or C-terminus of a protein. Alternatively, appended can refer to a modification within a protein (i.e., an insertion, or inserted, which can be used interchangeably).

It was specifically demonstrated that, a synthetic gene to alter the Toll-like Receptor (TLR) and Interleukin-1 Receptor (IL-1R) signaling pathway induced the expression of interferon-family cytokines. These findings indicated that synthetic genes are capable of inducing a strong interferon-based antitumor response.

Without wishing to be bound by theory, the invention described herein creates a means to induce robust anti-tumor immunity. Work over the last several years has established that anti-tumor immunity occurs naturally, and that tumors that are more immunogenic are better treated by current T-cell based immune-therapies. In particular, the efficacy of several cancer therapies is highly correlated with the expression of interferon genes in the tumor. As such, it was hypothesized that a means to increase the expression of interferons in the tumor environment should enhance anti-tumor immune responses.

In some embodiments, this approach may also increase the spectrum of cancers that can be treated with immuno-therapy. Current approaches to increase tumor immunogenicity have focused mainly on the injection into the tumor of cytokines or microbial activators of inflammation. Many technologies have been developed based on this idea, and the invention described herein does not follow this model. Rather, a unique approach was taken to induce tumor immunogenicity. This approach was based on the idea that naturally existing signaling pathways could be rewired within tumors to force these cells to induce an immunogenic transcriptional response when these cells were exposed to the signals present in the natural environment.

In a preferred aspect, synthetic biology was capable to generate novel genes that rewire the signaling pathways of the immune system. In particular examples, a gene to alter the Toll-like Receptor (TLR) and Interleukin-1 Receptor (IL-1R) signaling pathways have been developed such that they induced the expression of interferon-family cytokines. Of note, many tumors have been documented to be naturally exposed to ligands that activate these receptors-however these receptors were not designed to couple ligand binding to the expression of interferons. Thus, these synthetic genes can be introduced into tumors and to identify whether the natural IL-1R or TLR ligands that the tumor experiences promote an interferon-based antitumor response.

In certain aspects, the immune response can be rewired in a wide range of cancers. Development of diverse synthetic gene therapies for a wide range of cancers, including lung, breast, prostate and rare cancers, is contemplated.

Combined with the high unmet clinical need for cancer therapies, the discoveries of the instant invention warrant clinical translation as both a unique antitumor immunity therapy. The current discovery that synthetic genes can be used, rather than conventional techniques (i.e., antibodies, small molecules and the like), distinguishes the currently described invention and exemplified uses of the synthetic genes from previous suggestions regarding the antitumor response.

### Current Therapies Using Synthetic Biology

Most therapeutics are designed to either activate or interfere with a biological process of interest. However, the growing field of synthetic biology offers another possible path for therapeutic development-through the “rewiring” cells to induce unnatural (synthetic) responses. These synthetic responses may provide therapeutic opportunities to treat certain diseases. For example, by creating a synthetic immune signaling pathway that combines the beneficial activities of separate pathways, we may be able to better stimulate anti-tumor immunity. These powerful approaches to study signal transduction have rarely been utilized to study the innate immune system.

Anti-tumor immunity depends on our ability to stimulate inflammatory pathways in the tumor micro-environment that recruit immune cells that promote antigen specific immunity and cells that kill tumor cell bearing those antigens. Central to these stimulatory events are inflammatory cytokines, interferons, and cell death responses. While these activities are important for anti-tumor immunity, yet none are induced by a single signaling pathway. Synthetic biology approaches should allow for the design of a signaling pathway within tumor cells or other diseases tissue environments that induces a combination of these activities, and consequently more effective immuno-therapy.

### Definitions

Unless defined otherwise, all technical and scientific terms used herein have the meaning commonly understood by a person skilled in the art to which this invention belongs. The following references provide one of skill with a general definition of many of the terms used in this invention: The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise

As used herein, the singular forms “a”, “an”, and “the” include plural forms unless the context clearly dictates otherwise.

Unless specifically stated or obvious from context, as used herein, the term “or” is understood to be inclusive

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

By “activate” is meant an increase in activity, level, or other measurable parameter relative to a reference (i.e., an untreated control). Such activation can be by about 10%, 25%, 50%, 75% or more.

“Administering” is defined herein as a means of providing an agent or a composition containing the agent to a subject in a manner, which results in the agent being inside the subject's body. Such an administration can be by any route including, without limitation, oral, transdermal (e.g., vagina, rectum, oral mucosa), by injection (e.g., subcutaneous, intravenous, parenterally, intraperitoneally, intrathecal), or by inhalation (e.g., oral or nasal). Pharmaceutical preparations are, of course, given by forms suitable for each administration route.

“Cancer” as used herein, can include the following types of cancer, breast cancer, biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Other cancers will be known to one of ordinary skill in the art.

As used herein, the terms “comprises,” “comprising,” “containing,” “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Concurrently administered” as used herein means that two compounds are administered sufficiently close in time to achieve a combined immunological effect. Concurrent administration may thus be carried out by sequential administration or simultaneous administration (e.g., simultaneous administration in a common, or the same, carrier).

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “gene” is meant a locus (or region) of DNA that encodes a functional RNA or protein product, and is the molecular unit of heredity.

“Immunogen” and “antigen” are used interchangeably and mean any compound to which a cellular or humoral immune response is to be directed against. Non-living immunogens include, e.g., killed immunogens, subunit vaccines, recombinant proteins or peptides or the like.

The adjuvants of the invention can be used with any suitable immunogen. Exemplary immunogens of interest include those constituting or derived from a virus, a mycoplasma, a parasite, a protozoan, a prion or the like.

The “modulation” of, e.g., a symptom, level or biological activity of a molecule, or the like, refers, for example, to the symptom or activity, or the like that is detectably increased or decreased. Such increase or decrease may be observed in treated subjects as compared to subjects not treated with an adjuvant lipid of the invention (a non-canonical inflammasome-activating lipid), where the untreated subjects (e.g., subjects administered immunogen in the absence of adjuvant lipid) have, or are subject to developing, the same or similar disease or infection as treated subjects. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or within any range between any two of these values. Modulation may be determined subjectively or objectively, e.g., by the subject's self-assessment, by a clinician's assessment or by conducting an appropriate assay or measurement, including, e.g., assessment of the extent and/or quality of immunostimulatory response in a subject achieved by an administered synthetic gene of the invention (e.g., a MyD88 synthetic gene containing the pLxIS motif). Modulation may be transient, prolonged or permanent or it may be variable at relevant times during or after an adjuvant lipid of the invention is administered to a subject or is used in an assay or other method described herein or a cited reference, e.g., within times described infra, or about 12 hours to 24 or 48 hours after the administration or use of a novel, synthetic gene of the invention to about 3, 4, 5, 6, 7, 8, 9, 10, 11, 12, 13, 14, 21, 28 days, or 1, 3, 6, 9 months or more after a subject(s) has received such an immunostimulatory composition/treatment.

A “motif” as used herein can refer to a peptide sequence of any length, but in particular embodiments can be from two to about 300 amino acids in length. In some examples, the motif may be thought of as peptide sequences that define a portion (i.e., domain) of the protein having or directing a specific function such as, e.g., the reactive site of an enzyme, structural elements (α-helix, β-sheet, etc.), or a binding site for a ligand or regulator or signal of the protein.

By “neoplasia” is meant any disease that is caused by or results in inappropriately high levels of cell division, inappropriately low levels of apoptosis, or both. For example, cancer is an example of a neoplasia. Examples of cancers include, without limitation, pancreatic cancer, including islet cell and adenocarcinomas), duodenal cancers, cholangiocarcinomas, ampullary tumors, leukemia's (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colorectal carcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, neuroendocrine carcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “nucleic acid” is meant biopolymers, or large biomolecules, essential for all known forms of life. Nucleic acids, which include DNA (deoxyribonucleic acid) and RNA (ribonucleic acid), are made from monomers known as nucleotides. Each nucleotide has three components: a 5-carbon sugar, a phosphate group, and a nitrogenous base. If the sugar is deoxyribose, the polymer is DNA. If the sugar is ribose, the polymer is RNA. Together with proteins, nucleic acids are the most important biological macromolecules; each are found in abundance in all living things, where they function in encoding, transmitting and expressing genetic information—in other words, information is conveyed through the nucleic acid sequence, or the order of nucleotides within a DNA or RNA molecule. Strings of nucleotides strung together in a specific sequence are the mechanism for storing and transmitting hereditary, or genetic information via protein synthesis. Nucleic acids include but are not limited to: deoxyribonucleic acid (DNA), ribonucleic acid (RNA), double-stranded DNA (dsDNA), single-stranded DNA (ssDNA), messenger RNA (mRNA), ribosomal RNA (rRNA), transfer RNA (tRNA), micro RNA (miRNA), and small interfering RNA (siRNA).

By “nucleic acid sequence” is meant a succession of letters that indicate the order of nucleotides within a DNA (using GACT) or RNA (GACU) molecule. By convention, sequences are usually presented from the 5′ end to the 3′ end. For DNA, the sense strand is used. Because nucleic acids are normally linear (unbranched) polymers, specifying the sequence is equivalent to defining the covalent structure of the entire molecule. For this reason, the nucleic acid sequence is also termed the primary structure. The sequence has capacity to represent information. Biological DNA represents the information which directs the functions of a living thing. In that context, the term genetic sequence is often used. Sequences can be read from the biological raw material through DNA sequencing methods. Nucleic acids also have a secondary structure and tertiary structure. Primary structure is sometimes mistakenly referred to as primary sequence.

As used herein, “nucleic acid molecule” or “polynucleotides”, refers to a polymer of nucleotides. Non-limiting examples thereof include DNA (e.g., genomic DNA, cDNA), RNA molecules (e.g., mRNA) and chimeras thereof, e.g., encoding the loop C peptide of SEQ ID NO: 6. The nucleic acid molecule can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the term “nucleic acid” and polynucleotides as are analogs thereof. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Intl Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see The Biochemistry of the Nucleic Acids 5-36, Adams et al., ed., 11th ed., 1992), or known derivatives of purine or pyrimidine bases (see, Cook, PCT Int'l Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs). An “isolated nucleic acid molecule”, as is generally understood and used herein, refers to a polymer of nucleotides, and includes, but should not limited to DNA and RNA. The “isolated” nucleic acid molecule is purified from its natural in vivo state, obtained by cloning or chemically synthesized.

As used herein, “nucleotide” is used as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, e.g., Usman and McSwiggen, supra; Eckstein, et al., International PCT Publication No. WO 92/07065; Usman et al, International PCT Publication No. WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). By “modified bases” in this aspect is meant nucleotide bases other than adenine (A), guanine (G), cytosine (C), thymine (T), and uracil (U) at 1′ position or their equivalents.

The term, “percent (%) amino acid sequence identity” or “homology” with respect to a protein. The homology or percent amino acid sequence identity may be defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. Alignment for purposes of determining percent amino acid sequence identity can be achieved in various ways that are within the skill in the art, for instance, using publicly available computer software such as BLAST, BLAST-2 or ALIGN software. Those skilled in the art can determine appropriate parameters for measuring alignment, including any algorithms needed to achieve maximal alignment over the full length of the sequences being compared.

As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. Pharmaceutically acceptable carriers include sterile aqueous solutions or dispersions and sterile powders for the extemporaneous preparation of sterile topical solutions or dispersion. The use of such media and agents for pharmaceutically active substances is well known in the art.

The terms “polypeptide”, “peptide”, “amino acid sequence” and “protein” are used interchangeably herein to refer to polypeptides of amino acids of any length. The polypeptides may be linear or branched, it may comprise modified amino acids, and it may be interrupted by non-amino acids. The terms also encompass an amino acid polypeptides that has been modified, for example, disulfide bond formation, glycosylation, lipidation, acetylation, phosphorylation, or any other manipulation, such as conjugation with a labeling component. As used herein, the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

A “signaling protein” or “protein that elicits a response” or “targeting protein” can be referred to, but are not limited to, a protein, an enzyme, an adaptor protein, a membrane protein, a receptor, and the like that can induce a signal or, alternatively, can receive a signal. Exemplary signals may include are not limited to, intracrine signals, autocrine signals (i.e., from the immune system), signals in the inflammatory pathway (e.g., the inflammasome), metabolic pathways, and the like. Additionally, the synthetic genes described herein can induce or suppress cell division, differentiation, and cell-cell communication, and migration, phagocytosis, and the like. In some examples, the signaling protein may be an adaptor protein (i.e., MAVS, STING, or TRIF and the like). Alternatively a protein that does not induce the response may include any protein that does not elicit any of the above-mentioned responses (i.e., from the immune system, signals in the inflammatory pathway (e.g., the inflammasome), metabolic pathways, and the like.

As used herein, “subject” includes animals that possess an adaptive immune system, as described herein, such as human (e.g., human subjects) and non-human animals. The term “non-human animals” includes all vertebrates, e.g., mammals, e.g., rodents, e.g., mice, and non-mammals, such as non-human primates, e.g., sheep, dog, cow, chickens, amphibians, reptiles, etc. . . . .

A “suitable dosage level” refers to a dosage level that provides a therapeutically reasonable balance between pharmacological effectiveness and deleterious effects. For example, this dosage level can be related to the peak or average serum levels in a subject of, e.g., an anti-immunogen antibody produced following administration of an immunogenic composition (comprising a synthetic gene of the invention) at the particular dosage level.

As used herein, the terms “treat,” treating,” “treatment,” and the like refer to reducing or ameliorating a disorder and/or symptoms associated therewith. It will be appreciated that, although not precluded, treating a disorder or condition (i.e., a cancer) does not require that the disorder, condition or symptoms associated therewith be completely eliminated.

The terms “tumor,” “solid tumor,” “primary tumor,” and “secondary tumor” refer to carcinomas, sarcomas, adenomas, and cancers of neuronal origin and, in fact, to any type of cancer which does not originate from the hematopoietic cells and in particular concerns: carcinoma, sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, retinoblastoma, multiple myeloma, rectal carcinoma, thyroid cancer, head and neck cancer, brain cancer, cancer of the peripheral nervous system, cancer of the central nervous system, neuroblastoma, cancer of the endometrium, as well as metastasis of all the above.

By “variant” it is meant that a sequence described herein differs in at least one amino acid position from the wild type sequence. By way of example, “variant” pLxIS motifs may indicate that the pLxIS motif differs in at least one amino acid position from the wild type pLxIS motif.

Unless specifically stated or obvious from context, as used herein, the term “or” is understood to be inclusive.

Unless specifically stated or obvious from context, as used herein, the terms “a”, “an”, and “the” are understood to be singular or plural.

Any compositions or methods provided herein can be combined with one or more of any of the other compositions and methods provided herein.

### Supramolecular Organizing Centers (SMOCs).

The ability to detect and respond to environmental stresses represents one of the key features of living organisms. In the context of host-pathogen interactions, the innate immune system provides a faithful illustration to this principle of life, as failure to rapidly sense or respond to pathogens would cast a fatal stress on the host (Pandey et al., 2014).

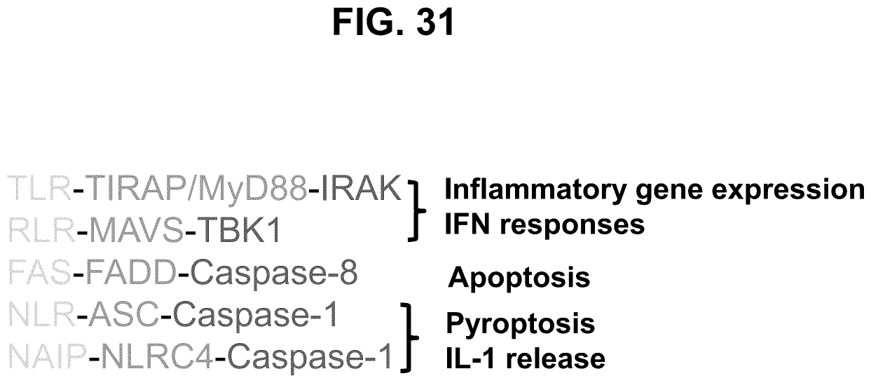

The signaling organelles of the innate immune system consist of oligomeric protein complexes known as supramolecular organizing centers (SMOCs). Supramolecular Organization Centers (SMOCs) consist of Receptor-Adaptor-Effector Protein Complex (FIG. 31). Each is assembled upon ligand/microbe encounters. Examples of SMOCs include the myddosome, the inflammasome, and the RIG-I-MAVS complex, which respectively regulate TLR-, NLR-, and RLR-mediated responses. The common use of these oligomeric structures as signaling platforms suggests multifunctionality, yet each SMOC has a singular biochemically-defined effector function.

Herein, it is reported that the myddosome is a multifunctional organizing center. In addition to promoting inflammatory transcription factor activation, the myddosome drives the rapid induction of aerobic glycolysis. The kinase TBK1 was identified as a novel myddosome component, which is dedicated to glycolysis induction. Synthetic immunology approaches further diversified myddosome activities, as this SMOC was engineered to induce interferon production or necroptosis downstream of TLR activation. These discoveries demonstrate the multifunctionality of an immune signaling organelle and highlight SMOCs as modular and programmable signal transduction platforms.

Pattern Recognition Receptor (PRR) Signaling

Pattern recognition receptors (PRRs) are a primitive part of the immune system. They are proteins expressed by cells of the innate immune system to identify two classes of molecules: pathogen-associated molecular patterns (PAMPs), which are associated with microbial pathogens, and damage-associated molecular patterns (DAMPs), which are associated with cell components that are released during cell damage or death.

PRRs are classified according to their ligand specificity, function, localization and/or evolutionary relationships. On the basis of function, PRRs may be divided into endocytic PRRs and signaling PRRs. Signaling PRRs include the large families of membrane-bound Toll-like receptors (TLRs) and cytoplasmic NOD-like receptors. Endocytic PRRs promote the attachment, engulfment and destruction of microorganisms by phagocytes, without relaying an intracellular signal. These PRRs recognize carbohydrates and include mannose receptors of macrophages, glucan receptors present on all phagocytes and scavenger receptors that recognize charged ligands, are found on all phagocytes and mediate removal of apoptotic cells.

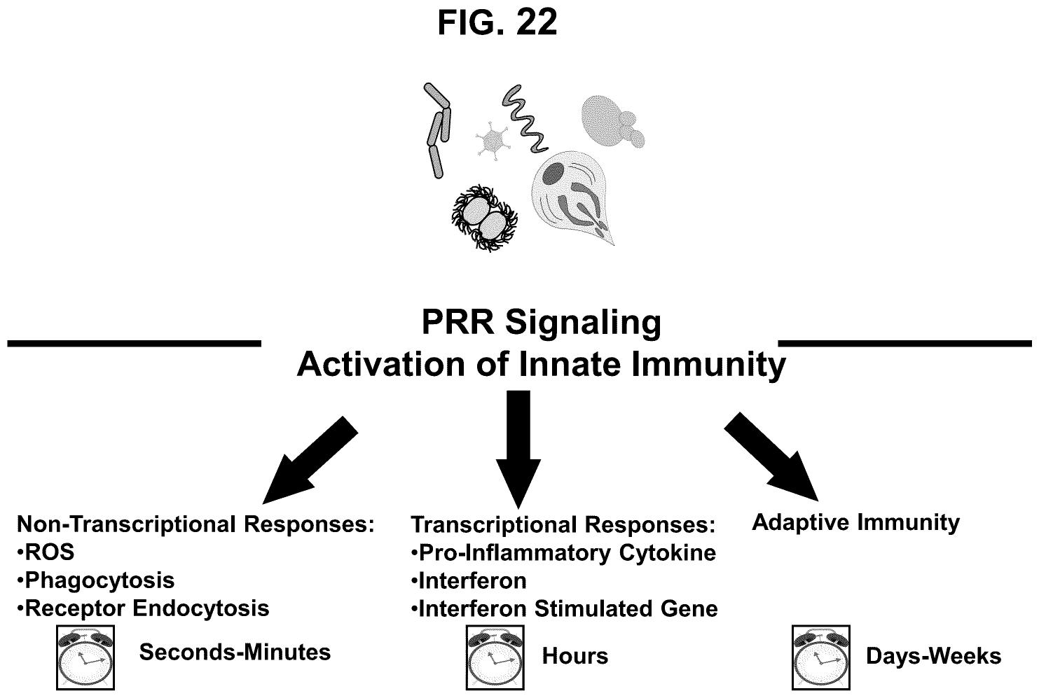

A variety of host responses are controlled by PRR signaling (FIG. 22) For example, within seconds to minutes, exemplary non-transcriptional responses are activated and may include ROS, phagocytosis and receptor endocytosis. Within hours, transcriptional responses may be activated, which include pro-inflammatory cytokine, interferon, or interferon stimulated gene responses. Adaptive immunity is activated within days to weeks.

Myddosome: Generally, the myddosome can be thought of a complex of signaling proteins with a role in immune response. The myddosome functions as a signaling platform to coordinate diverse cellular processes upon TLR activation. In addition to the well-characterized transcriptional responses, TLR pathway activation has also been implicated in diverse host responses such as metabolic reprogramming, autophagy ROS production cell death etc., which are non-transcriptional responses. Whereas myddosome formation is known to activate NF-kB activation, it is largely unclear whether myddosome formation induces these other responses. Therefore, the myddosome was used a signaling platform that coordinated diverse cellular processes upon TLR activation.

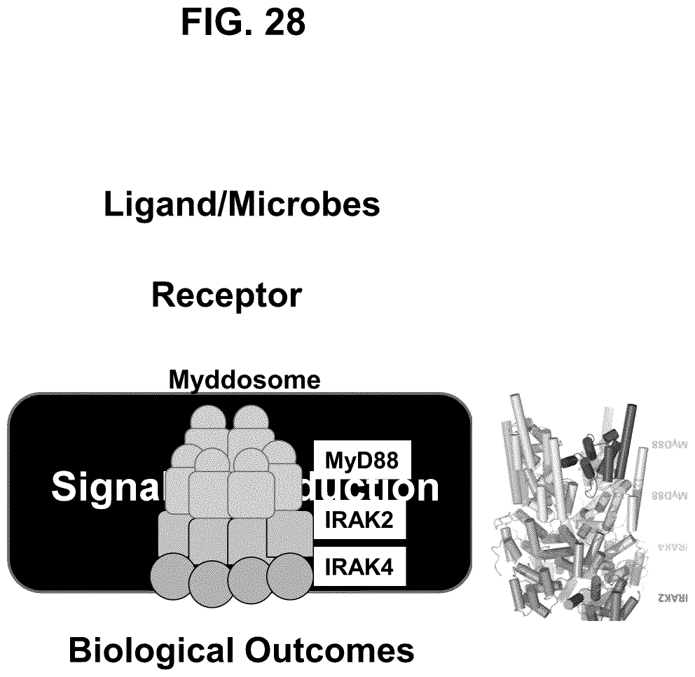

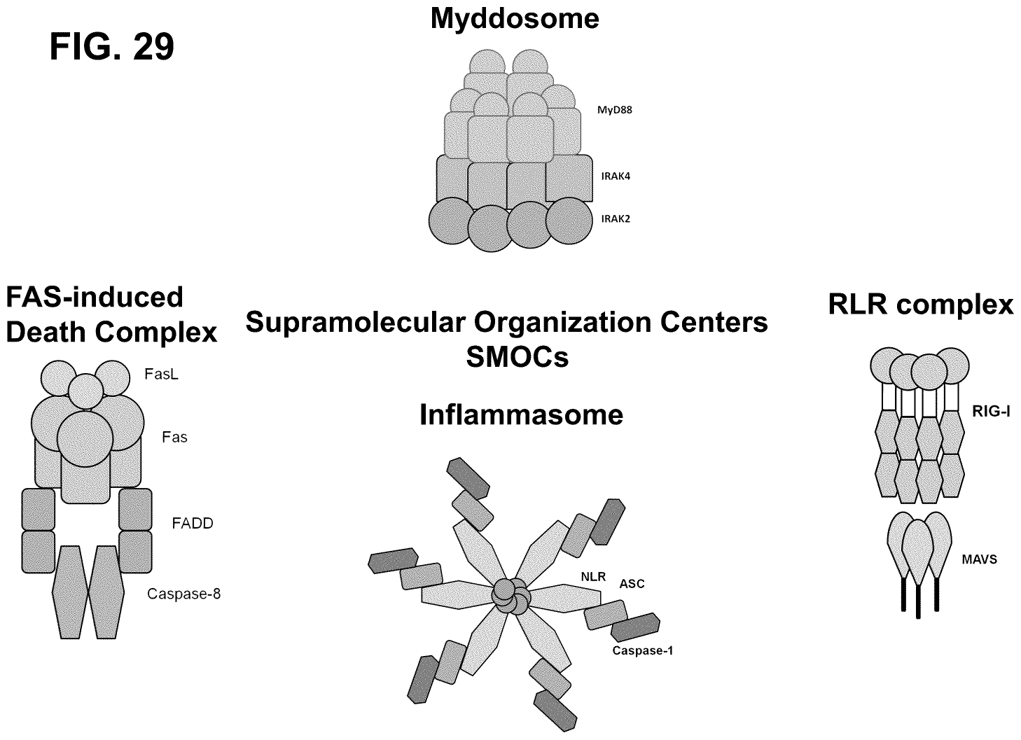

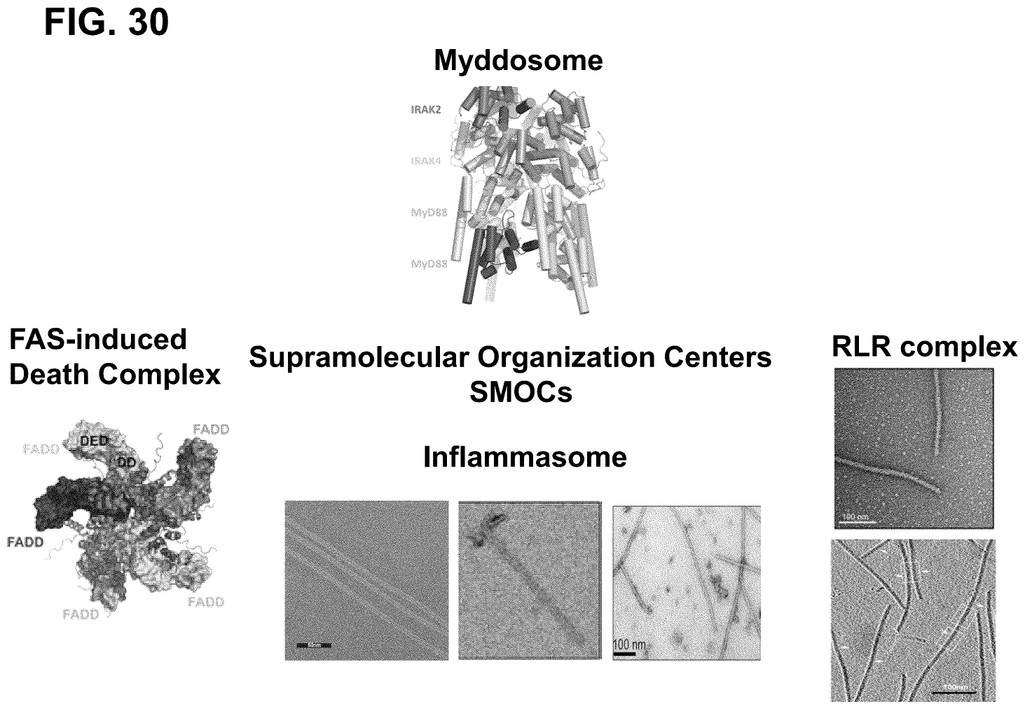

The structural study of myddosome, a protein complex composed of MyD88 and IRAK kinases described these findings. Myd88 form higher order helical structures with downstream IRAK family kinases which leads to a drastic increase of kinase concentration at a local area to propagate downstream signaling (FIG. 28-30). Note that Myd88 is the signaling adaptor for all the TLRs except TLR3. From a structural biology perspective, these higher-order helical structures serve as platforms to recruit effector molecules such as kinases to amply and propagate downstream signaling events. Furthermore, a signaling paradox facing the Toll like receptor family is that, they are not enzymes, they are without enzymatic activities, and therefore, how do these receptors induce robust host responses in the presence of limited ligands.

Toll-Like Receptors: Toll-like receptors (TLRs) are type I transmembrane receptors, evolutionarily conserved between insects and humans. Ten TLRs have so far been established (TLRs 1-10) (Sabroe, I. et al., (2003) Journal of Immunology 171(4): 1630-5). Members of the TLR family have similar extracellular and intracellular domains; their extracellular domains have been shown to have leucine-rich repeating sequences, and their intracellular domains are similar to the intracellular region of the interleukin-1 receptor (IL-1 R). TLR cells are expressed differentially among immune cells and other cells (including vascular epithelial cells, adipocytes, cardiac myocytes and intestinal epithelial cells). The intracellular domain of the TLRs can interact with the adaptor protein Myd88, which also possess the IL-1 R domain in its cytoplasmic region, leading to NF-KB activation of cytokines; this Myd88 pathway is one way by which cytokine release is affected by TLR activation. The main expression of TLRs is in cell types such as antigen presenting cells (e.g. dendritic cells, macrophages etc.). One such TLR is TLR4, which is responsible for activating the innate immune system and recognizes lipopolysaccharide (LPS), a component of gram-negative bacteria. TLR4 has been shown to interact with lymphocyte antigen 96, Myd88 (myeloid differentiation primary response gene 88), and TOLLIP (toll interacting protein).

Activation of dendritic cells by stimulation through the TLRs leads to maturation of dendritic cells, and production of inflammatory cytokines such as IL-12. Research carried out so far has found that TLRs recognize different types of agonists, although some agonists are common to several TLRs. TLR agonists are predominantly derived from bacteria or viruses, and include molecules such as flagellin or bacterial lipopolysaccharide (LPS).

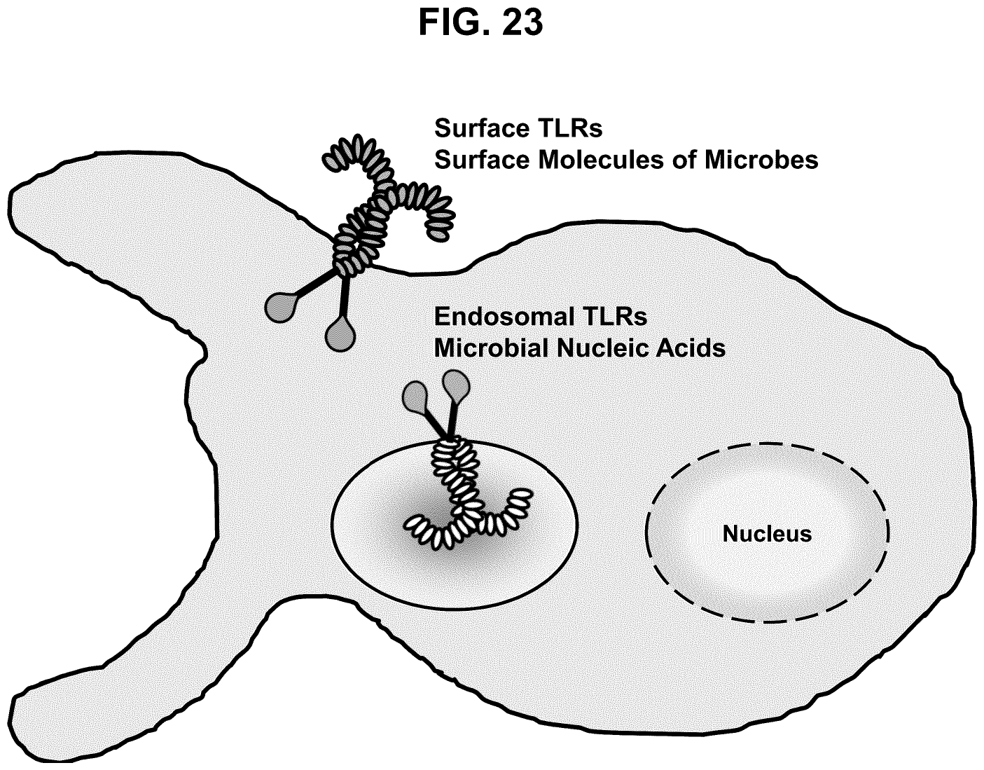

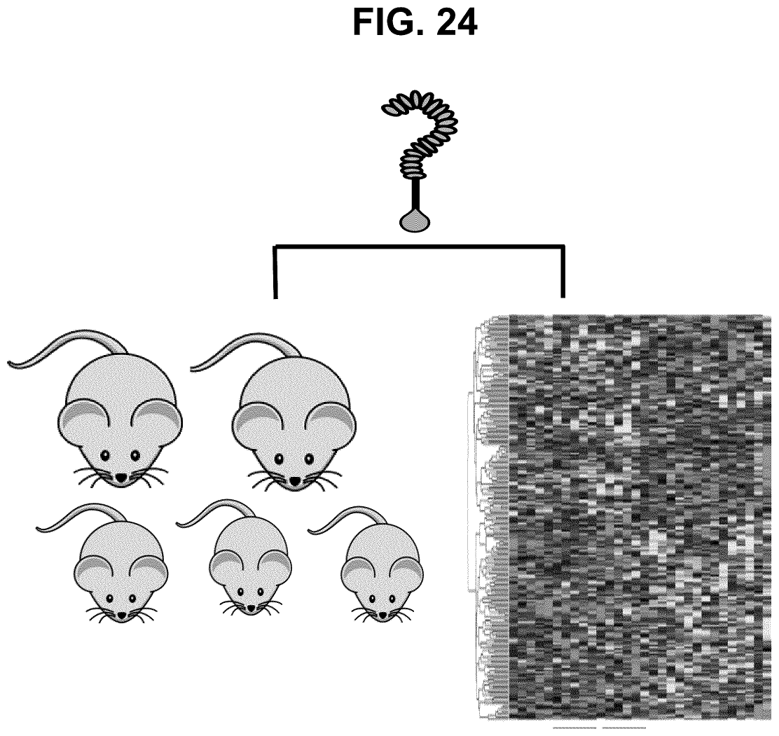

The TLR family is one of the best genetically defined PRR families (FIGS. 23-24). The technical advances in genetic sequencing, proteomic, and transcriptomic has led to the identification of hundreds of host proteins in the TLR pathway, and the knowledge of the host transcriptional responses has been refined to a single-cell level. This progress lays the foundation to study the cellular biological and biochemical functions of the host proteins in the pathway.

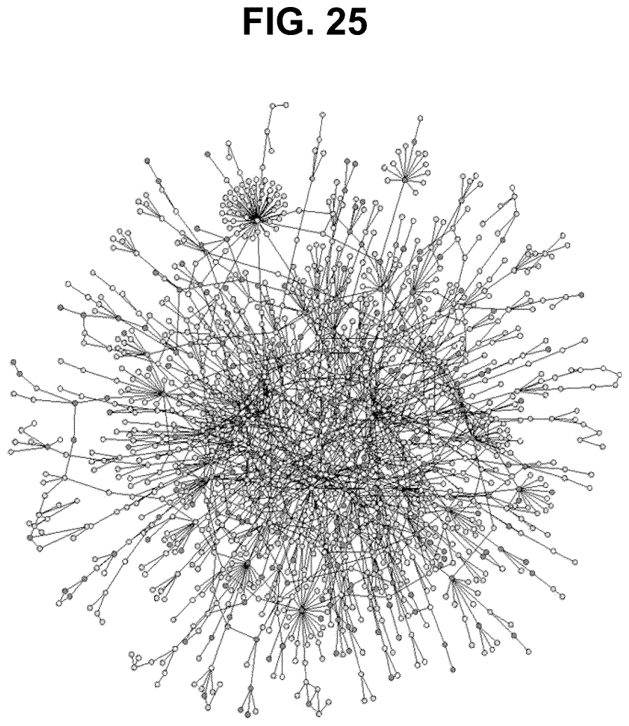

A common challenge is understanding how different host components work in space and time during microbial encounters (FIG. 25). This knowledge allowed for utilization of the power of the innate immune system. For example, the knowledge has allowed boosting of the pathway to fight infectious agents, to fine-tune the pathway for vaccination, or to cut the pathway to treat immunopathologies such as sepsis.

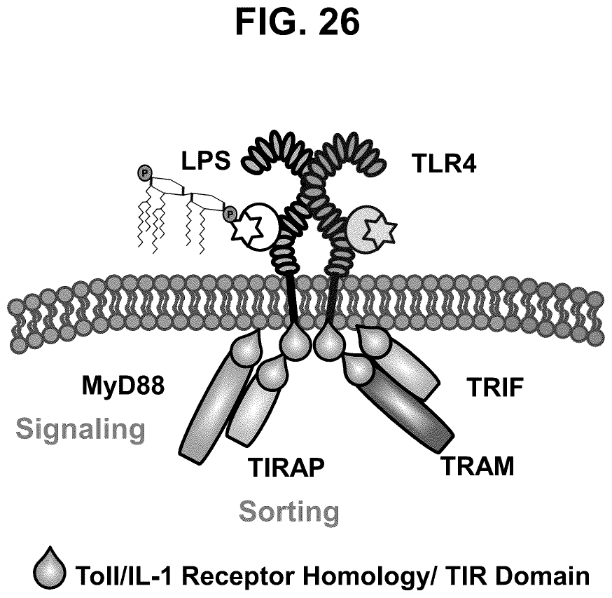

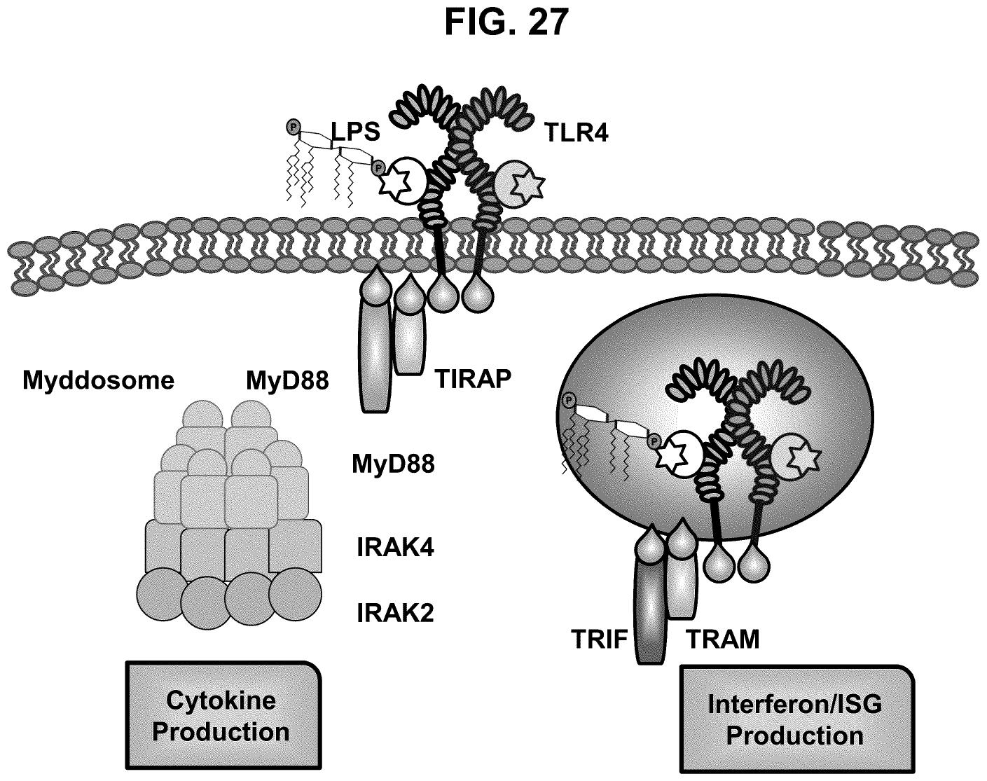

Proteins in the TLR pathway operate in a coordinated manner (FIG. 26). Upon activation, TLR4 engages four cytosolic adaptor proteins containing the Toll/IL-1 Receptor Homology domain (TIR). TIR domain containing adaptor proteins could be functionally categorized into sorting adaptors and signaling adaptors, by which sorting adaptors link the activated receptor to signaling adaptors to trigger host response (FIG. 27).

Immediately downstream of receptor trafficking and upstream of transcriptional responses within the nucleus, lies the process that is known as signal transduction. A signaling paradox facing by the Toll like receptor family is that they are not enzymes and without enzymatic activities, how these receptors induce robust host responses in the presence of limited ligands is questioned.

TLR superfamily and synthetic biology: The Toll-like Receptor (TLR) superfamily (which includes the IL-1 (IL-1) receptor family) is a strong inducer of inflammation and a common target for immuno-therapeutics. However, this pathway does not have the ability to robustly induce interferon or cell death responses. Similarly, the inflammasome pathways are strong inducers of cell death and inflammation, but cannot activate interferon responses. A specific domain within the innate immune regulatory proteins STING, MAVS and TRIF are recognized to be important for the activation of an interferon response, yet this domain is absent in the regulatory proteins that function most TLR and IL-1 signaling pathways. Three examples of this principle are described below to highlight the general strategy that is beneficial for immune-therapy.

By engineering a synthetic gene where the interferon inducing domain (known as pLxIS) is fused to the coding sequence of the TLR/IL-1 pathway regulator MyD88, a hybrid protein will be produced that has the unique ability to induce cytokines and interferons.

By engineering a synthetic gene where a death inducing domain (known as RHIM) from the kinase RIPK3 is fused to the coding sequence of MyD88, a hybrid protein will be produced that has the unique ability to link TLR/IL-1 activation to cell death.

By engineering a synthetic gene where the pLxIS is fused to the coding sequence of the inflammasome regulator ASC, a hybrid protein will be produced that has the unique ability to link inflammasome assembly to interferon expression.

Other analogous strategies are foreseen, but these examples provide an overview of the approach to create synthetic immune signaling pathways that combine beneficial activities into one.

MATVS: The terms “mitochondrial antiviral-signaling protein,” “MAVS,” “VISA,” “virus-induced signaling adapter,” “IPS-1,” and “Cardif” as used herein refer to an intracellular adaptor protein encoded by the MAVS gene. In embodiments, the terms refer to a polypeptide or fragment thereof having at least 85%, 90%, 95%, 99%, or more amino acid identity to NCBI Accession Nos. Q7Z434, Q7Z434.2, and NP 065797.2.

The exemplary sequence at NCBI Accession No. Q7Z434 is:

| | | | | | | |
|---|---|---|---|---|---|---|
| 1 | mpfaedktyk | yicrnfsnfc | nvdvveilpy | lpcltardqd | rlratctlsg | nrdtlwhlfn |
| 61 | tlqrrpgwve | yfiaalrgce | lvdladevas | vyqsyqprts | drppdplepp | slpaerpgpp |
| 121 | tpaaahsipy | nscrekepsy | pmpvetqap | espgenseqa | lqtlspraip | rnpdggples |
| 181 | ssdlaalspl | tssghqeqdt | elgsthtaga | tssltpsrgp | vspsysfqpl | arstprasrl |
| 241 | pgptgsvvst | gtsfsssspg | lasagaaegk | qgaesdgaep | iicssgaeap | anslpskvpt |
| 301 | tlmpvntval | kvpanpasvs | tvpsklptss | kppgavpsna | ltnpapsklp | instragmvp |
| 361 | skvptsmvlt | kvsastvptd | gssrneetpa | aptpagatgg | ssawldssse | nrglgselsk |
| 421 | pgvlasqvds | pfsgcfedla | isastslgmg | pchgpeeney | ksegtfgihv | aenpsiqlle |
| 481 | gnpgppadpd | ggprpqadrk | fgerevpchr | pspgalwlqv | avtgvlvvtl | lvvlyrrrlh |

The exemplary sequence at NCBI Accession No. Q7Z434.2 is:

| | | | | | | |
|---|---|---|---|---|---|---|
| 1 | mpfaedktyk | yicrnfsnfc | nvdvveilpy | lpcltardqd | rlratctlsg | nrdtlwhlfn |
| 61 | tlqrrpgwve | yfiaalrgce | lvdladevas | vyqsyqprts | drppdplepp | slpaerpgpp |
| 121 | tpaaahsipy | nscrekepsy | pmpvqetqap | espgenseqa | lqtlspraip | rnpdggples |
| 181 | ssdlaalspl | tssghqeqdt | elgsthtaga | tssltpsrgp | vspsysfqpl | arstprasrl |
| 241 | pgptgsvvst | gtsfsssspg | lasagaaegk | qgaesdgaep | iicssgaeap | anslpskvpt |
| 301 | tlmpvntval | kvpanpasvs | tvpsklptss | kppgavpsna | ltnpapsklp | instragmvp |
| 361 | skvptsmvlt | kvsastvptd | gssrneetpa | aptpagatgg | ssawldssse | nrglgselsk |
| 421 | pgvlasqvds | pfsgcfedla | isastslgmg | pchgpeeney | ksegtfgihv | aenpsiqlle |
| 481 | gnpgppadpd | ggprpqadrk | fqerevpchr | pspgalwlqv | avtgvlvvtl | lvvlyrrrlh |

The exemplary sequence at NCBI Accession No. NP-065797.2 is:

| | | | | | | |
|---|---|---|---|---|---|---|
| 1 | mpfaedktyk | yicrnfsnfc | nvdvveilpy | lpcltardqd | rlratctlsg | nrdtlwhlfn |
| 61 | tlqrrpgwve | yfiaalrgce | lvdladevas | vyqsyqprts | drppdplepp | slpaerpgpp |
| 121 | tpaaahsipy | nscrekepsy | pmpvqetqap | espgenseqa | lqtlspraip | rnpdggples |
| 181 | ssdlaalspl | tssghqeqdt | elgsthtaga | tssltpsrgp | vspsysfqpl | arstprasrl |
| 241 | pgptgsvvst | gtsfsssspg | lasagaaegk | qgaesdgaep | iicssgaeap | anslpskvpt |
| 301 | tlmpvntval | kvpanpasys | tvpsklptss | kppgavpsna | ltnpapsklp | instragmvp |
| 361 | skvptsmvlt | kvsastvptd | gssrneetpa | aptpagatgg | ssawldssse | nrglgselsk |
| 421 | pgvlasqvds | pfsgcfedla | isastslgmg | pchgpeeney | ksegtfgihv | aenpsiqlle |
| 481 | gnpgppadpd | ggprpqadrk | fqerevpchr | pspgalwlqv | avtgvlvvtl | lvvlyrrrlh |

By “MAVS,” “VISA,” “IPS-1,” “Cardif,” and the like are meant a polynucleotide encoding a MAVS polypeptide or fragment thereof (e.g., a polynucleotide encoding the amino acid sequence of NCBI Accession Nos. Q7Z434, Q7Z434.2, and NP-065797.2). An exemplary sequence is provided at NCBI Accession No. NC-000020 (Gene ID: 57506

| | |
|---|---|
| 1 | acatagccaa tggccgcgcg ctctgcccgc cccgcctcct cgctgcggga agggtcctgg |
| 61 | gccccgggcg gcggtcgcca ggtctcaggg ccgggggtac ccgaggtaag atcgcttccc |
| 121 | gggcgttggg tcctttcggc tcagcacgca cggacgcctt tagggaaggt ggctgcagcg |
| 181 | gcaggacgga gtccgccggg acgccctggg tctggggtgc gcggggggcc caggagggga |
| 241 | caggacgcgc ggggatccgg aagagcgggc cctgtcgcaa gagtttcggg aacactgagg |
| 301 | gctccaggcg cggcatccag gatccgggga aaagggggta ggtggcgctg ggcgtctgct |
| 361 | caggctgggg gaaaggtagg gccagaaggg gacgggcagc ggcggctgac ctcctcctgc |
| 421 | cgcccgcggg cccagggtga cgctaaggtg gggccgagcc tcgaccgggt gcgcctagag |
| 481 | gtcgagtgct gccgcgctcc gctgggtctg gacagttctc ggcggcgaca ccagctcaaa |
| 541 | acggcctccc cgccctccgc ggacctgggt cgcgcccagg aatccgatcc aaggctgtga |
| 601 | ggcctgtccc tttgggaagg gtgggtgttt atttccggga tgcactcaga gcgtctggac |
| 661 | agtcaggtcg gaaactttgc tgtattggga acactctgtc acctacttcc ttctcagttg |
| 721 | ggaaggaagt gccaagaaaa catgaaacaa accaaaaaca cgaaaaaggg attctctgta |
| 781 | tggaagccgt gaagcctcaa aaatatctag gaggacagcc agcgacctgg gacctgtggc |
| 841 | agccatgtga aagcagggtt aatgtctgga ctaaatgttg cttccaccta agtgcaccct |
| 901 | cagcctccct cccgccaagt gaccttgggt cctctttggg cttgaaggca ggtggctgtg |
| 961 | tgggtctcgc tgcaggggtc tctgtgccct gcaaggtgta tgaccagttg cagtgaggca |
| 1021 | gcaggttttg ggttcaaatc ctgtgctagc ctctggccag ctgtgcacct tggcaacact |
| 1081 | ttccctgtca cgggattggg gaaggattaa ctgaaagaac cttaggatgt gcctgtctac |
| 1141 | aatgggccct ccataaatgt gaacaaatgt gggcttcctt tccttttgtt tgggccacat |
| 1201 | catcccttcc cctccatctg tggctgaagc tggaatgcag aagagtgcct catctgactg |
| 1261 | ccttctggta cctggctgat gccatgagaa aggaaggaga aaggggtctt tttttttttg |
| 1321 | aaatggagtc tcactctctt gcccaggctg gagcgcagtg gtccaatctt ggctcactgc |
| 1381 | aacctctgcc tcctgggtct agagattctc ctgcctcagc ctcctgagta gctgggacta |
| 1441 | caggtgtgtg ccaccatgcc tggctaattt ttgcattttt agtagagacg gggtttcacc |
| 1501 | atgttggcca ggctggtctc aaactcctga cctgaagtga tctgcctgcc tcggcctccc |
| 1561 | aaagtgctgg gattccaggc gtgagccacc gggcccggcc gagaaaggga tctattaact |
| 1621 | cccatagagt tgttctttgc taatttcttg aaggctcaga ggacccccgc ctcaccttcc |
| 1681 | tgattctcct gacctgtcat tagtacttgc cccacgagga atgtagcagg gcctgctggc |
| 1741 | tggcaaagca actcatgcat gtgaggctct gaggccagtg acaggactgc ttcccctgtg |
| 1801 | aggaaggtct ggtggcccaa cagcttttag gtgctgtctg ctctacagca ctgcctcctg |
| 1861 | agagaggtct catgcctgcc tgatgcccac ttggtcctct cctgcctcct tccctccctg |
| 1921 | acaacccact tggaatccaa tagcatctca aacttcactt gttccgaact gagttctgga |
| 1981 | gtcccttctg agccactgct cctcccctgg cttctctggc ctggtaaaag ggcaccttcc |
| 2041 | atccacccag tgcccaagga gtcatgcttc ttttctctcc cttatctcct acaccctcaa |
| 2101 | aagaccagga atctggctgc ctcctgccat ctctgtggtt cccatcctga ccatagtcat |
| 2161 | cctgtctcct gggctgtggc ctccttactg gtctcccagt tttcatcctg gcccctccaa |
| 2221 | agtcctcaca accaccagag aagtctttaa tgtaaatcag atcctcttct ttccctgccg |
| 2281 | gaaccttcca gtggttccct gtttcactcc aactaaaacc cagagtcctt tcctacagca |
| 2341 | ctctacatga gtggcccctg ccacctcctt gaccttgaca atctctgccc ctttccctag |
| 2401 | cttgcttgct tttttttttt tttttcctat gatggagttt tgatcttgtc acccaggctg |
| 2461 | gagtgcaatg ggatgatttc agctcactgc aacctctgcc tcctgggttc aagcgattct |
| 2521 | cctgcctcag cctcccgagt agctgggatt acaggcgccc accaccatgc taatttttgt |
| 2581 | atttttagta gagaaagggt ttcaccatgt tggcgaggct ggtctcaacc tcctgagctc |
| 2641 | aggtgatcca cccgcctagg cctcccaaag tgctgggatt ataggcgtga gtcacgcagc |
| 2701 | cagccatccc tagctttctt gacctagacc acactgacct gctttctatt cttcaaacat |
| 2761 | gccaagctca ttcttgtttt aggacttttg catttaccat gctctctgcc taaaacacca |
| 2821 | atcttctcag agcctgagaa cagctcagct gtgttctgca cgctagttca gaaaggcttc |
| 2881 | tttgaccccc tagttcaagt agcatgcctg tgtccagggg ctctgtctca ttacctgctt |
| 2941 | tactttcttt agagccttta ttgctatctg gaactcttat ttgggtatta atttactgat |
| 3001 | ctgttatttg tcccacccca ttagaatata aattggatgt ggcatagacc ttgtctcttt |
| 3061 | tattccctgc agcactccct gatgggggcg gcagaaaagt aacgagcaaa tacatctata |
| 3121 | actgcaaatt gtggtaactc ctacataaac aactggcagc aattgcttat atttgaggca |
| 3181 | cttaaaaatt tttaagctgg ctgggcgcgg tggctcatgc ctgtaatccc agcactttgg |
| 3241 | gaggccgagg cgggcagatc acgaggtcag gagatcgaga ccatcctggc taacacggtg |
| 3301 | aaacctgtct ctactaaaaa tacaaaaaat tagccgagcg tggtagcagg cgcctgtagt |
| 3361 | cccagctact tgggaggctg aggcaggaga atggtgtgaa cccgggaggc ggagcttgca |
| 3421 | gtgagccgag attgcaccac tgcactccag cctgggtgaa ggagcgagac tgtctcaaaa |
| 3481 | aaaaaaaaaa aaaaaaaaaa agaaattttt ttaagctgct gggcgcggtg gctcacgcct |
| 3541 | gtaaatctca gcactttggg aggccgaggt gagcggatca cctgaggtcg ggagttcgag |
| 3601 | accggaacat ggtgaaaccc tgtctctact aaaaatacaa aattagcggg gcgtggtggc |
| 3661 | tcatgcctgt aatcccagct acttgggagg ctgaggcagg agaatcgctt gaacccagga |
| 3721 | ggcagaggtt gcagtgagcc gagatcgcgc cattgtactc cagcctgggc aaaaagagtg |
| 3781 | aactccattt caaaaaaaaa aaaaaaggcc aggcgcagtg gctcacgcct gtaatcccag |
| 3841 | cactttggga ggccgaggca ggcggatcac gagttcagga gattgagacc atcctggcta |
| 3901 | acacggtgaa accctatctc tactaaaaat acaaaaaatt agccgggtgt ggtggcgggc |
| 3961 | gcctgtggtc ccagctactc gggaggctga ggcaggagaa tggtgtgaac ccaggaggtg |
| 4021 | gagcttgcag tgagccgaga ttgcaccaca gcactccagc tttggtgaca gagcgaaact |
| 4081 | ccgtctcaaa aaaaaaaaaa aaaaaaattt aagcttagag gccggccaca gtggctcagc |
| 4141 | actgtgcagg ccaaggcaag aggatcactt gaggtcaaga gttcgagacc agcctggcca |
| 4201 | acatggtgaa accctgtctc tactaaaaaa tacaaaaatt ggccaggcgc gttggctggc |
| 4261 | gcctgtaatc ctagcaactt gggagaccaa ggcaggcaga tcacctgggg tcaggagttc |
| 4321 | aggaccggcc tggccaacat taaaacatat aaaaccccgt ctctactaaa aatataaaaa |
| 4381 | ttatccaggc atggtggcgt gtacccgtaa tcccagctac tcgggaggct gaggtaggag |
| 4441 | aattgcttaa acccgagaag cagaggttgc agtgaaccga gattacgcca ctgcactcca |
| 4501 | gcctgggcaa cagagcgaga ctctttctca aaaacaacaa caacaacaaa caaacaaatt |
| 4561 | agccaggcat gatggtgggc acgtgtaatc ccagctactc gggaggctga ggcaggagaa |
| 4621 | ttgcttgaat gtgggagatg gaggctgcag tgagccgaga tcacaccact gcactccagc |
| 4681 | ctgggcgaca gggagactct gtctcaaaaa aaaaaaaaaa aaaaaaagtt tataaggctg |
| 4741 | aattaccgta ctgtcaaaac aagctgctat ctgagccgtt ttaagggtga ggaagtctgg |
| 4801 | aaactgataa cttgcccagg acacacagtg agttcaaggc atggaactca gtctcctatc |
| 4861 | ttaagaatgt atgtgggccg ggcatggtgg gtcacgcctg taatcccagc gctttgggag |
| 4921 | gccaaggcag gcagatcatc tgaggtcagg agttcaagac cagcctgacc aacatggaga |
| 4981 | aaccctgtct ctactaaaaa tacaaaatta accaggtgtg gtggtgcatg tctgtaattc |
| 5041 | cagctactca ggaggctgag gcagaagaat cacttgaacc cggaaggcag aggttgcgat |
| 5101 | gagccgagat tgtgccattg tactccagcc tgggcaacaa gagtctggaa ctctgtctca |
| 5161 | aaaaagaaaa aaagaatgta tgtgtagcag gctttttttt tttttttttc ccccgagacg |
| 5221 | gaatctggct ctgtcgccca ggctggagtg gagtggcgca atgttggctc actgcaagct |
| 5281 | ccgcctccca ggttcacgcc attctcctgc ctcagcctcc cgagtagctg ggactacagg |
| 5341 | cacccgccag tacgccgggc taattttttg tatttttagt agagacgggg tttcaccgtg |
| 5401 | ttagccagga tggtcttgat ctcctgacct cgtgatccac ccgcctcggc ctcccaaagt |
| 5461 | gctgggatta caggcgtgag ccaccgtgcc cggcctatgt gtagcaggct ttaatggtgg |
| 5521 | gcctgcagcc atgtcatgga aagaagctga cctgaagatc tcagttcttt cttcttctac |
| 5581 | taactagcaa gcatacctca gtttcttctt taaagcggga tgatccgatt attatcatgt |
| 5641 | tggggttcac tttttatttt ttcagtgtgt cccaaagcag cagcacgttt aggtatagcc |
| 5701 | ctcttgctat cagcttgagg gccttagagc caggaaggga gccaggacat ttataggcac |
| 5761 | agaaactagg gtcacataca gatcccccca ccgcatgtgc taggggtaca tgcagacctt |
| 5821 | cccagtgctg accaacctgc agagaagaaa tgggccctag gtattctgga tctgattctt |
| 5881 | tttggtcttc aattattttt atttttattt ttttagagac agggtctcgc tgtgttgccc |
| 5941 | aggctggcct cgaacagctg ggctcaagcg atcctcctgc cctagcttct tgagtagctg |
| 6001 | gtggtcatca attcattttt agcaaattct gcagaatttt tttttttttt tttttttttg |
| 6061 | agacggagtc tcactctgcc gcccaggctg gagtgcagtg gcgtgatctc ggctcactac |
| 6121 | aacctccgcc tcttgggttc aagcaattct ctgtctcagc ttcctgaata gctgggactg |
| 6181 | caggcgcccg ccaccatgct tggctaattt ttttgtattt tcagtagaga cggggtttca |
| 6241 | ccatcttggc caagttggta ttgaactcct gacctcgtga tccatccgcc tcggcctccc |
| 6301 | aacgtgctgg ggttacaggc gtgagccacc gcgcccgggt tctgcaggaa ttttggagag |
| 6361 | actcaggcag taataaaata ggatgtttac agaaattaaa gatggcggcc gggcgcggtg |
| 6421 | gctcacgcct gtaatcccag cactttggga ggccgaggcg ggcgcatcac gaggtcagta |
| 6481 | aatcgagacc atcctggcta accccgtgaa accccgtctc tactaaaata caaaaaaatt |
| 6541 | agccgggcgt ggtggcgggc gcctgtcgtc ccagctactc aggaggctga ggcaggagaa |
| 6601 | tggcgtgaac ccgagaggcg gagcttgcag tgagccgaga tcgcgccacc gcactccagc |
| 6661 | ctgggcgaca gagaaagact ccgtctcaaa aaaaaaaaaa agaaattaaa ggtggctgga |
| 6721 | cacattggct ggtgcttgtc atccgagcta cttgacaggc ggaggcaggg ggatcgcttg |
| 6781 | aggccaggcg tttgagacca gcctgggcag catcatgaga ccctgtctct agaaaaaata |
| 6841 | aaaaaattag ctgggcatag tggcgcaggt ttgtagttcc agctaccggg gatgctgagg |
| 6901 | cgggaggatt gcttgagccc acgagttcga ggctgcagtg aactattatt gcaccactgc |
| 6961 | acccaacttg ggtgacagag accccatctg tttgtttgtt tgtttttgag acagagtttc |
| 7021 | gctcttgttg cccaggctgg agtgcaatgg tgcaatcttg gctcaccgca acctctgccc |
| 7081 | ccaggttcaa gcaattctcc tgcctcaacc tcccgagtag ctgggattac aggcatgcgc |
| 7141 | caccatgccc agctattttt tttttttttt tgtattttta gtagagacgg gattttctcc |
| 7201 | atgttggtca gtctggtctc caactcccga cctcagttaa tcccccaaat tggcctccca |
| 7261 | aagtgctggg attataggcg tgaaccactg tgcccagccc gagaccccat ctcttaaaaa |
| 7321 | caaaataaaa caaaacaaaa acggccaggt gtggtggctc acacctgtaa tccccaaact |
| 7381 | tgggaggccg aggcgggtgg accacttgag gtcaggagtc tgtgaccagc ttgccaacat |
| 7441 | ggtgaaaccc catctctact aaaaatacaa aaattagctg ggcatggtgg tgcgcacctg |
| 7501 | taatcccagc tactcagaag ggaggctgag gcaagagact caattgaacc caggaggcgg |
| 7561 | aggttgcagt gagccgagat tgccccactg cactccagcc tgggtgacaa agtgagactc |
| 7621 | gctctgaaaa aaaaaaaaaa gaagaaatta aagatgaaag aaaacaaaca ttccaaaaag |
| 7681 | ttgagaaaga attgcctttt gtccagcccc actcccaacg ccccaaccct gttgtaatgt |
| 7741 | gtgatctgtt ttcttccagt ctcgtttcct ctcagtccat ccacccttca tggggccaga |
| 7801 | gccctctctc cagaatctga gcagcaatgc cgtttgctga agacaagacc tataagtata |
| 7861 | tctgccgcaa tttcagcaat ttttgcaatg tggatgttgt agagattctg ccttacctgc |
| 7921 | cctgcctcac agcaagagac caggtgagca agggaagtga cagcccgaca ctggcctggg |
| 7981 | ggcagggctg tggaattcaa agctcagccc catcctagtt cctcacccaa gcctgggctg |
| 5041 | gctccttcct tcttcctctt gctgtgtctt gctccttgtc cttgctgctt ttcttttttt |
| 8101 | tttttttttt tgagattgag tctcgttctg tcgccaggct ggagtgcagt ggcacgatct |
| 8161 | tggctcattg caacctccgc ctcctgggtt caagtgattc tcctgcctca gcctcctgag |
| 8221 | tagctgggat tacaggtgcg tgccaccacg cccagctaat ttttttgttt ttaatagaga |
| 8281 | cggggtttca ccatgttggc caggatggtc ttgatctctt gaccttgtga tccgcctgcc |
| 5341 | tcggcctccc aaagtgctgg gattacaggc gtgagccacc gcacccttgc tgcttttcta |
| 8401 | acttttggat ggagtgtggc tcagggtggc gttgctgact tcgccgagct cccccttgtg |
| 8461 | ttgcttttgt gcactgctca aaaatatggc gctggctctc tgagatttcc tggctctggt |
| 8521 | ccacttgccc actttttttg gaacctccta tttccttcat ctctcttgcc cttccttgtc |
| 8581 | ctgctcagtt ttgattccat tctccttgtc atggggccct gtcctggcac ggagctggga |
| 8641 | ctcaggtttg agagctggca ggatcagggt cgctctagcc ccaacagaac ttgctgcagg |
| 8701 | cccctggcac tcactagctg gtgaaacggg cacaacccct ccccgttgta gctgctgttc |
| 8761 | tcagattgga cccctgtgct ccagagggta cctgttggct cttttggggc ctcctgtcct |
| 8821 | cagatttctc aggagcccca ttgttgtctc cgctgtcctc ccacacagat cgcattagta |
| 8881 | tgcaggtctg tttggagttt gctcctccct cttgtatttt ggggtttata gggatatctt |
| 8941 | gttttatagt aaatattttc tgtgggtttt ctttattttc tttaaaaaat ttttttttga |
| 9001 | gacggagtct cgctgtgttg cccaggctgc agtgcaatgg catgatctca gctcactgca |
| 9061 | acctctgcct cctgggttca agtgattctc gcgcctcagc ctcctgagta gctggggtta |
| 9121 | caggcgcatg ccaccacacc tggctgattt tgtatttgta gtagagatgg agtttcacca |
| 9181 | tgttggccag gctggtcttt atttttattt ttgagacaga gtcttgctct gtcactgagg |
| 9241 | ctggagtgca gtggcacgat tttttttttt ttttgagacg gagtctcact ctgtcgccca |
| 9301 | ggctggagtg cagtggtgtg atctcggctc actgcaagct ctgcctcctg ggttcacgcc |
| 9361 | attctcctgc ctcagcctct tgagtagatg ggactacagg cgcctgccac catgcccggc |
| 9421 | taattttttg tatttttaat agagacgggg tttcactgtg ttagccagga ttgtctcgat |
| 9481 | ctcctgacct catgatccac ccgcctcggc ctcccaaagt gctgggatta caggcgtgag |
| 9541 | ccactgcgcc cagcattttt tttttttttt tttttgagat ggagtctcgc tgtgtcttcc |
| 9601 | aggctggagt tgcagtggtg ccatcttggg tcaacctctg cctcctgggt tcaagcaatt |
| 9661 | ctcctgcttc agcctcctga gtagctggga ttacaggtat atgctaccac acccggctaa |
| 9721 | tttttgtgtt tttagtagag acggactttc accatgttgg tcaggctggt cttgaactcc |
| 9781 | tgaccttgtg atcctcggcc ttccaaagtg ctgggattac gggtgtgagc taccgcacct |
| 9841 | ggctattttc ctttttctaa aaatctagct cctgcaggat tctgtgggtt tttgtttctg |
| 9901 | ctgtctggtt gcttgttttt atgtgagaat tcaggtagac ataaaaactc tagggctggg |
| 9961 | cacggtggct cacgcctgta atcccagcgc tttgggaggc caaggcgggt ggatcacctg |
| 10021 | aggtcaggag ttcgagacca gcctggccaa catggcgaaa ccatgtctct actaaaaata |
| 10081 | caaaaaaatt agccgggtgt ggtggtgggc tcctgtaatc ccagctactc gggaggctga |
| 10141 | ggcaggagaa tcgcttgaac tcaggaggca gaggttgcag taagctgata tcacggcact |
| 10201 | gcactccagc ctgggcgacg gagtgggact ccgtctgaaa aaaaaaaaaa aaaaagaaac |
| 10261 | aaaaaaactc tgcagccact gtcatctgcc cacaatctcc ccagcattct cagcttcctt |
| 10321 | gtttgttatt gtcggccccc tctctttccg tcttttgccc ctttcatcat acttttgcta |
| 10381 | tctacctttt ccttctctcc taatccaaac ctttattttt gccctggggg ccatattaat |
| 10442 | ccaaggcttt tgtatcagat taactgggtt tggattcctg ccccactgtt ttaggatctt |
| 10501 | tgctagagta ctttgcttct gctaagcctg agtttcctca ttagtaaagt ggagataata |
| 10561 | atggcattaa ataaagatga tacatgcaaa gcccttaatg gagagcccag gacatagtta |
| 10621 | attgccagtt tccggcaggt gcctttattg atgtggctgc taattgctct tcctgactgc |
| 10681 | atacctggcc ctgtcctggg ctccgatcca gtttcacgtg gctgccttgc ccttgtggct |
| 10741 | ttcttggcac ccctcccccc gctgtggctt cattctgggt ggggaagtgg caggggccac |
| 10801 | ctggcttgag caggacagtg gcattgtgtc ttccaggatc gactgcgggc cacctgcaca |
| 10861 | ctctcaggga acggggacac cctctggcat ctcttcaata cccttcagcg gcggcccggc |
| 10921 | tgggtggagt acttcattgc ggcagtgagg ggctgtgagc tagttgatct cgcggacgaa |
| 10981 | gtggcctctg tctaccagag ctaccagcct cgtgagcgtc ctgcccttgc cctcctggac |
| 11041 | ccccagcctg ctccctggcc tccgctctcc ttttctctct ccctgtactt cctgcctttc |
| 11101 | tctgtcatcc tctttcttgt cactgtgaag cgatgaataa acctgggtgt agatccaggc |
| 11161 | tgagccactt accagctgtg tccctttggc caagtccctt aatttccctg agcctcaggc |
| 11221 | ctctcttctg taaaatgaag ctcatggcag catctgccgc ggggagctgc agtgggtgat |
| 11281 | actgcgggac gatgcgtgtt gagtattgag ctgggctggg cacttcctgt atgcccagca |
| 11341 | catggagtct cccctaactt tcacggctgt agcattcgcc tcccaccctt cctcatttct |
| 11401 | tctcccccac ctactcattc accctccctc tctcctcctt ctcttcccct cccctggttt |
| 11461 | accctgagag ccttcgacgc cctctatcag ctgcccagtt attctttaag tccctctcag |
| 11521 | tgtccctgcc actctgagtg ctcggaggcg atttgatgag attgagtttg atcctgagtg |
| 11581 | agatcaagac atgggaggag gctgggcgcg gtgtttcaca cctgtaatcc cagcactttg |
| 11641 | ggaggccgag gcaggcggat catgaggtca ggagatggag accaccgtgg ctaaaacagt |
| 11701 | gaaaccccgt ctctactaaa aatacagaaa attagccggg catgttgtcc cagctactca |
| 11761 | ggaggccgag gcaggagaat cacttgaacc agggaggcag aggttgcagt gagctgagat |
| 11821 | cgcgccactg cactccagcc tgggcgacag agtgggattc catctcaaaa aaaaaaaaaa |
| 11881 | aaagacatgg gaaaaaaaat caagccagcc ctatttatat ttcaaactag aggtaacccc |
| 11941 | cgagaccctg gtcacattta tagctgtggg acatccatgt ttttcttttc tttctctctc |
| 12001 | tttttttttt ttccttttag agacagagtc ttgctgcgcc acccaggctg cagtgcagtg |
| 12061 | gtgcaatcat agctcactgc agccttgacc tcctggactc aagtgatcct tctacctcag |
| 12121 | cctccagagt agctgggact acaggcatgg acaactacac ctggctaatt tttaaatttt |
| 12181 | ttgtagagat gacatctcac tatgttgccc aggctggtct caaactcctg ggctgaagcg |
| 12241 | atcggcctcc cagagtgctg ggatcatagg tgtgagccac cgcgtctggc tctcatgctt |
| 12301 | gcttttctct cctttttccc ttccttgctt ttcctccctc cctccctccc ttcctctctt |
| 12361 | ccttgctttt tttccttcct tctttttaaa tatgtctctt catgtgtgga gattaatagt |
| 12421 | gatccctggc tgggcacggt ggctcacgcc tgtaatccca gcactttggg aggccgaggc |
| 12481 | gggcggatca caaggtcagg agttcgagac cagcctggcc aatatggtga aaccctgtct |
| 12541 | gtaccaaaaa tacaaaaaaa ttagctgcgc atggtggtgc aagcctgtaa tcccagctac |
| 12601 | ttgggaggct gaggcaggag aattgcttga accggggagg tggaggttgc agtgagccga |
| 12661 | gattgcgcca ctgcactcca gcctggatga cagagtgaga ctccgtctcc aaaaaaaaaa |
| 12721 | aacccaaaaa tagtgatccc ctgaatacaa tggctgtggt agggcctgat gaggggtggg |
| 12781 | ggcaaagggg aggggctcag gtggcagcat cagggcaggg gtcagtgagc aatgatagtc |
| 12841 | atatggagga gaaagccact gggtcctagg atgcctgggg acagagaaga gtgactgctg |
| 12901 | acacggcgtg ggtgactaga gaccgacgag gcccccccat agtccccttc ctcccttgct |
| 12961 | accttgtcct ccatctgctc tcaccctccc actcctgccc ccttgccaag tgatgcttgt |
| 13021 | cactcctttt ttttttgaaa tggagtttcg ctctgtcgcc caggctggag tgcagtggtg |
| 13081 | ccatctcagc tcactgcaag ctccgcctcc cgggttcacg ccattctcct gcctcagcct |
| 13141 | cccgagtagc tgggactaca ggcgcgtgca accatgcccg gctaactttt tgtatttttt |
| 13201 | agtagagatg gggtttcacc gtgttagcca ggatggtctc gatctcctga cctcgtgatc |
| 13261 | cacccgcctc ggcctcccaa agtgctggga ttacaggcgt gagccaccaa gcccagccct |
| 13321 | gcttgtcact cttgaggagt gggcccacat cagaacagct tttggaccta tgggtggggc |
| 13381 | ggggggtgta cccaagagca cccaagcctc tttaatcatg aggagaaccc ccaattcctt |
| 13441 | tttttttgag acagagtctt gctcagtcgc ccaggctgga gtgcagtggc atgacttcgg |
| 13501 | ctcaccacaa cctctgcctc ccgggttcaa gtggttctcc ttcctcagcc tccctatagt |
| 13561 | ccctgattcc ttctattttt tttttttttt tttgagacgg agtctcgctc ttgttgccca |
| 13621 | ggctggagtg caatggtgca atctcaggtc atggcaacct tcacttccca ggttcaagca |
| 13681 | attctcctgc ctcagcctct cgagtagctg ggattacagg catgcgcctc caggcctggc |
| 13741 | taattttgtt atttttagta gagacaaggt ttctccatgt tggtcaggct ggtctcgaac |
| 13801 | tcacgacctc aggtgatcca cccacttcgg cctcccaaag tgctgggatt acaggcgtga |
| 13861 | gccaccacgt ctggcttctt tttctttttt tcccccgaga cggagtcttg ctctgttgcc |
| 13921 | caggctggag tgcagtggcg cgatctcagc tcactgcaac ctccgtctcc caggttcaag |
| 13981 | caattcttct gcctcagcct cctgagtagc tgggattaca ggtgcttgcc agcacgcctg |
| 14041 | gctaattttt gtatttttag tagagacggg gtttcactat gttggccagg ctggtcttga |
| 14101 | actcctgacc tcctaatcca cctgccttgg cctccccaaa tcctgggatt acaggcatga |
| 14161 | gccatcgtgc ccagcccctg attccttctt tttttttctt tctttttttt tttagacgga |
| 14221 | gtctcgctct gtcgcccagg ctggagtgca gtggcgcgat cttggcttac tgcaagctcc |
| 14281 | gcctcccggg ttcacgccat tctcctgcct cagcctcctg agtagctggg actacagggg |
| 14341 | cccgccacca tgcccggcta ataataatgt tgtattttta gtagagatgg ggtttcactg |
| 14401 | tgttagccag ggtggtctcg atctgacctc gtgatctgcc tgccttggcc tcccaaagtg |
| 14461 | ctgagattac aggcatgagc cactgtgccc agccctgatt ccttcttgat atcactacat |
| 14521 | ctttgtcctc tagggacctc ggaccgtccc ccagacccac tggagccacc gtcacttcct |
| 14581 | gctgagaggc cagggccccc cacacctgct gcggcccaca gcatccccta caacagctgc |
| 14641 | agagagaagg agccaagtta ccccatgcct gtccaggaga cccaggcgcc agagtcccca |
| 14701 | ggagaggtct gtcctcatag tctaccttga gccaccactt ttgtgttcct atctgcccac |
| 14761 | ttctgcccat tgagccttcc agaaaccctc tcccgtcccc tataaatcac gcctaatctc |
| 14821 | tgctcagaac cctagggctt cctcagtggg gatctgcccc agaccagctt ccaggctgct |
| 14881 | gaccaggtct tcaccctgtg gcagccctaa tcctctgtca gcaaccagct gggagaccac |
| 14941 | agttttgtgt gtgtgtgtgt gtgtgtgtgt gacagtgtct cattctgtca cccaggctgg |
| 15001 | agtgcagtgg agtgatcttg gctcactgca acctctgcct cctgggttca ggtcattctc |
| 15061 | ctgcctcagc ctcctgagta gctgggatta caggcaccca ccaccacgcc cagctaattt |
| 15121 | ttgtattttt agtagagatg gggttttgcc gtgtcagcca ggctggtctc gaactcctga |
| 15181 | cctcaggtga tctgcccacc tttgcctccc aaagtgctgg gattacaggc gtgagccacc |
| 15241 | gcacctggca atgctgtgtg ttttctgtga ggtagacgta aggacacctg tggacagagg |
| 15301 | gtctgggaat taccagaacc caggcaaggg ctcccctggc tcctgtgctc catggtgtgg |
| 15361 | gctgaggcct ataggagatg ccccaagagc acaagctgcc ctttgtgagc tcttgggaga |
| 15421 | ggcaactgcc ttattcatat tttccctcat tgcagaattc agagcaagcc ctgcagacgc |
| 15481 | tcagccccag agccatccca aggaatccag atggtggccc cctggagtcc tcctctgacc |
| 15541 | tggcagccct cagccctctg acctccagcg ggcatcagga gcaggacaca gaactgggca |
| 15601 | gtacccacac agcaggtatg catggaatct ggaattatag ggtccttctg atctctcaag |
| 15661 | tgagggtaag aattagagtt gccccatctg gcttccttga acaggagaca aggtgggaat |
| 15721 | aaagggagtt caacccagga agcaaaccag ttccttagtg ggtgtatcag ttagcatttg |
| 15781 | ctgtgtaaca aatagtccaa tccagttttc caaatttttt ttttagtagc ttaaaataca |
| 15841 | gccatttatt tagcatatga tcctgtgggt caggcatttg ggctacctac atgggcattt |
| 15901 | cttctggtct tggctgaatt tcctctcaag tactcaccgg tatatacata agttctgcct |
| 15961 | ctggctgttt gctgagcacc ttggttctct tctatgtagt ctctcatcct ccagcacaca |
| 16021 | aacccatcat ggcagctggg cagagttctc agagagggct caaaactggc acagtgtccc |
| 16081 | ctgtgctcca ttctgtgggc aaaagcaagt tataaggcca gcctagattc aaggagtagg |
| 16141 | gaaatagact ccctccctag acgggaggac tgacaggcac agtgcagtgg ggctgggtgg |
| 16201 | agatgagcga gataagtagg gccatttttg cgctctgcca aagggactgt agggaacagc |
| 16261 | cagggcctat agggcagtgg gagagggaca gtgaagggct gcatcagctg ttggcagggg |
| 16321 | aacctttagg cactgtctta ccgcagagat ctccagttcc cagtgaatca tgaaaacttc |
| 16381 | tcagtcccca gaggaagtaa ggtcttcatc atccagtggc ctggactcaa ctccagatgt |
| 16441 | cagtgctccc cctcagaaat atatagttgt ccatctggac ctctcaggcc agcatgtctc |
| 16501 | tttcctactt cccaaactat tccacatgac gctggtgccc agtcagccct cagtgccctg |
| 16561 | ggacagccac aagacacatg agcagttaga ggctgggaga cgtcatctta gtacttttgt |
| 16621 | catccccaaa ctgctccaag cacctgtctg ctttgcagtg tcacctggcc acgggatgcc |
| 16681 | tttcaggagt tgctgtagac cacagaggca gagggcgctt aggtttcagt acgtttgtag |
| 16741 | acacaggtcc catgagattc tgtggtatta gattgtggtg ggggagctgt acatcagaat |
| 16801 | caccctgact tttgccagct gtggggcttg gcatgtgcat tccgagttcc gtggagagtc |
| 16861 | ctgctgcaac tgcctttaca gaccatcacc acctgctatc ctctgcttcc cccacccagg |
| 16921 | tcaggcagcc tcccaggggt ggctttgtcc ttgtcccctc tcttcccaag cctccgggat |
| 16981 | ggccaggcct ctcggctggt gtgagctgtt ctgcatgagc catcctgcca ccccttgccc |
| 17041 | tgatccatgg ctgctcccac tcatggtggt aggagaggga cagcagtggg ggaagtgtcc |
| 17101 | aggattgcat gaggctaagg tcaaagtaga aaaggtagac acaggagagg agaggtttcc |
| 17161 | caggtgggag aggaaaaagc ggagagaata attaataatg gtcttcaggc tcctaggtac |
| 17221 | catttcactg tgtgccagga cagacctggg gctacaggtc aaggactgag ggcagctgtt |
| 17281 | gggctttcag gccaggaagc agtgaccaaa gggactgtgg catctcctcc aagggcagga |
| 17341 | gatttggagg cctagacaca gtagggacca tgagatctgg gccagaggga cccttctcca |
| 17401 | ggcctcaagg taatggtctt tgggtctgtg tttccacttg tgtttttcca ccggcaggtg |
| 17461 | cgacctccag cctcacacca tcccgtgggc ctgtgtctcc atctgtctcc ttccagcccc |
| 17521 | tggcccgttc cacccccagg gcaagccgct tgcctggacc cacagggtca gttgtatcta |
| 17581 | ctggcacctc cttctcctcc tcatcccctg gcttggcctc tgcaggggct gcagagggta |
| 17641 | aacagggtgc agagagtgac caggccgagc ctatcatctg ctccagtggg gcagaggcac |
| 17701 | ctgccaactc tctgccctcc aaagtgccta ccaccttgat gcctgtgaac acagtggccc |
| 17761 | tgaaagtgcc tgccaaccca gcatctgtca gcacagtgcc ctccaagttg ccaactagct |
| 17821 | caaagccccc tggtgcagtg ccttctaatg cgctcaccaa tccagcacca tccaaattgc |
| 17881 | ccatcaactc aacccgtgct ggcatggtgc catccaaagt gcctactagc atggtgctca |
| 17941 | ccaaggtgtc tgccagcaca gtccccactg acgggagcag cagaaatgag gtgagtcctc |
| 18001 | gcccttcctg gcagggatcc tggccccttc ccccgggaca gcttgcccac ctggccctgg |
| 18061 | ccttggcccc ttcccagtct gcattctgtg tccagcctgt gctgctctgt ggcctctcct |
| 18121 | tgagggcata cagacagttg agaaccagcc tcatgcaggc cccacaccat gttctccagg |
| 18181 | aggaacagtc attgagcttc taagtctgga cacctcagga gggtcagcca cagggggcac |
| 18241 | ccactggtca ggtgtataag ttcatttagg gctcgtagtt cctagtgaag ccgagcggtg |
| 18301 | ccgttttgca cataaggaag cagtgacggg gacagcacag tggcccatct gcctcttgcc |
| 18361 | ttgctcttca ccaggatgcc tggtgtgtcc ctccatggcc aggctttaca gaacgcagtc |
| 18421 | ccacctggag cagccactcg gacccagcag ccccccattg ttgcctgctc caagcctcac |
| 18481 | atctaaccct agctgcggct gtctgctggg aagagccaag tccatagggc cctttgggca |
| 18541 | catggccagg cctctgaccc tgtggctgct ctctagttct caggcccagg caggatgtca |
| 18601 | gtgcaggatg gagccccgcc ctaccaaagg cttccaggtg ggcatgagct cacaggcagg |
| 18661 | ccagggagta gggaaaggct gccctggagg aggccaccat tggtgcagat tcttggtccc |
| 18721 | ctctaccccc actgctccaa gaaaaggtgg cctaggggca ttatagattg ggaattgagg |
| 18781 | ggttggagtg ttagttcatg ccctggcctg ggaatgggac cgccctacca ggttcgtctc |
| 18841 | cctgccaacc ccagtccctt ccagtgctct cctttctttc ccaggagacc ccagcagctc |
| 18901 | caacacccgc cggcgccact ggaggcagct cagcctggct agacagcagc tctgagaata |
| 18961 | ggggccttgg gtcggagctg agtaagcctg gcgtgctggc atcccaggta gacagcccgt |
| 19021 | tctcgggctg cttcgaggat cttgccatca gtgccagcac ctccttgggc atggggccct |
| 19081 | gccatggccc agaggagaat gagtataagt ccgagggcac ctttgggatc cacgtggctg |
| 19141 | agaaccccag catccagctc ctggagggca accctgggcc acctgcggac ccggatggcg |
| 19201 | gccccaggcc acaagccgac cggaagttcc aggagaggga ggtgccatgc cacaggccct |
| 19261 | cacctggggc tctgtggctc caggtggctg tgacaggggt gctggtagtc acactcctgg |
| 19321 | tggtgctgta ccggcggcgt ctgcactagt gaagccctgg gctcttccca ccacccatct |
| 19381 | gttccgttcc tgcagtacac ctggcccctc tccgaagccc cttgtccctt tcttggggat |
| 19441 | tgtggaggct gggtcagagg ggagttaagg gactgcaggc ctggcagcag gacatgcctt |
| 19501 | ggctgaacca agtcctgaga gcagcatctc tgtccccacg gtgccttgtg tgggtccccg |
| 19561 | tccttggctt tctgggtcct gggctgcccc cagtgctcca gaccttcccc actggcaatc |
| 19621 | caggttatca tccatgtcct ccagaggagc ttcctcctcc aggcctcagc cctgttggcc |
| 19681 | caggtggagc aggagggacc actggaacat gtggtgcttg ggaatgcctc tcctgttgca |
| 19741 | ttggtccctg aaggcctcag ggcaggtatg tggtgtgtgg gcgactccac aagacctgcc |
| 19801 | tcccatcctg gcagcccagc ctgagaccgt tgcattgagg caggcaggag cggcagggtg |
| 19861 | gctgctctcc aggagcccaa ctgccttgag ttcctgcccc actgggcccc ctcccctgct |
| 19921 | gggcaatcct gggaaggtct ggaggttcct gtggacctca gggaagccag gggcagctgt |
| 19981 | caggcctgag gaagacctgt ggagctcctc tccagcctcc tctttccctc ccctctggtc |
| 20041 | tccattctct tcagctccct acatgggctg gggaggagac acctggtggg cagagctcag |
| 20101 | gcagaggttt ggatttcagc tccctcactt ccggggctgt gtggctttgg cagatgtcag |
| 20161 | acttctggtc ttgcttctcc acgtggacag tgagtatctg gctcattctt cactgggttc |
| 20221 | ttctgagatt gaacctacag gtgtttgcca agtgcctggc ccagagcaag tggccactgc |
| 20281 | ttctcccatc tctctcctgc ccaacctggt agagctgagg gcatgagagg cagagtgcac |
| 20341 | agtggtcaag ggtgcagctc tgcagcacag gcagcctagg cctgcgtccc aacctgcctc |
| 20401 | tcaccagctc tgtgaccttg ggcaagggat ttatctgtct gtcccttagt tttctcacct |
| 20461 | gtaaaaggag gataagtata tatatatatt tcccagtgtt gtgaagatta aaggagttta |
| 20521 | tcgatgtagg tcttaggatg agtcctggca tttaccaagg gttggatata tgttattatc |
| 20581 | actattaagt gttgagggtc caggcatgct gggcaacagg gacaccatct ctacaaaaaa |
| 20641 | gtttaaaaaa ttagccaggc gtggtggtgc acctgtcgtc ttagctactt gggaggctga |
| 20701 | ggtgggagga tcacttgagc ccagaagctt gaagctgcag tgagctagga tcgtgccact |
| 20761 | gcactccaac ctgggtgaga gagcgagacc ctgtctcaag aaaaagaaaa atgcagagaa |
| 20821 | acaggagtct tggctactcc tttagaggca gactcagacc ctcctgcctc acagctttat |
| 20881 | ctttgtattt gccccttact ttatcttgtg ccttgagaaa ttgctgggga gcgaggtatg |
| 20941 | tccactgggc agctgtacag gatggaggat atagggcgtt tccactccca ggagccaggt |
| 21001 | tccctcaccc caagctcacc cactgttggg gagattatct acaataacac cagaaacaca |
| 21061 | ttggggtgga ttgggggtat ccttatgggt tcttttcagg gaaccattgc tggacaaggc |
| 21121 | acaggagcca cttccatttc tgagctctgc aagggacaag aactagaggc atcaggggct |
| 21181 | gggctcactg tggccccacc ccaagccgtc aggctccagg gatctacacc ctgccttggc |
| 21241 | tgctacagct ttttcactcc actgccctag gggagttcag caacctaatg atctctatct |
| 21301 | ctgaacatct cttcatccca tgctcgaagt ccagcaacct gcaccctgga accaggagtg |
| 21361 | gaccctaccc gagctgtctg tattaatccc catcccccac caccaatctt aaaaagccct |
| 21421 | ctgtccccct accctaaacc ccagttaggt acccatgctg ggcaggtcag ttaacaattt |
| 21481 | atgcacaggt actagtttta ttgtattacc gttccagggt agctttgaaa aaagtatctc |
| 21541 | aaaaaggcaa catgggccga gcgcagtggc tcacgcttgt aatcccagca ctttgggagg |
| 21601 | ccaaggtggg cagatcgcct gaggtctgga gttcaagacc agcctggcca acagggtgaa |
| 21661 | accccgtctc tacaaaaata agaagattag ccaggtgtag tggcagacgt ctgtaatccc |
| 21721 | agctattcag gaggctgagg cacgagaatt ccatgaaccc aggatgcgga ggttgcagtg |
| 21781 | agccgagatt gtgccactgc gctccagctt gggcgacaga gtggtattct gtttcaaaaa |
| 21841 | aaaaaaaaaa ggcagtatgt agccccgaag actgttgccc aagtggtaga atgttagcac |
| 21901 | actaccagcc taggtaaaaa atacaaaaag taactgggca tggcggcgcc catctatagt |
| 21961 | cccagctaca tgggaggctg aggtgggaag ataagtcact tgagcccgcc aggaggcgga |
| 22021 | ggttgtagtg agctgagatc gcaccactgc agtccagcct gggtgaccga gtgatactct |
| 22081 | gtctcaaaga caaaaaatta taattttagc acagtaacca gccatgatgg gagataccct |
| 22141 | gggtaaggca tgtagaaagg gttgagggac cttcccagtc ccctagcccc gcctgccatc |
| 22201 | ctcccatctt tttctttttt ctttttttta gagaatcacc cagcctggag cgaagtggtg |
| 22261 | caatcataac tcactgtatc cttaaactcc cgggcttaag cgatcctcct gcctcagcct |
| 22321 | tctgagtaac taggacttca ggtacctgtc accatgcctg gctaattaaa tttttttttc |
| 22381 | tttttttttt ttgagatgga gtcttgctct gtcaccgagg ctggagtgca gtggcgcgat |
| 22441 | ctcagctcac tgcgacctcc agcctccggg ttcaggctat tctcccgcct cagcctccag |
| 22501 | agtagctggg actacaggcg cctgccacca cgcctggcta atttttttgc acttttagta |
| 22561 | gagacggggt ttcactgtgt tagccaggat ggtctcgatc tcctgacctt gtgatccgcc |
| 22621 | cgcctcggcc tgccaaagtg ctgggattac aggcgtgagc caccgggccc agccaaatta |
| 22681 | aattttttat agagatgagg tcatgctgtt atgttggcca ggttggcccg atgagatctt |
| 22741 | gccttagcct cccaaagtgc tgggattaca gatgtgagac actgcaccca aaccccacca |
| 22801 | cttttttttt tcctttttct ttttttgaga cagtcttact ccgttgccca ggctggagtg |
| 22861 | tagtggcatg atctcagctc actgcaacct ccgcctcccg ggttcaagca attctcctgc |
| 22921 | ctcagcctcc cgagtagctg ggattacaga ggcctgccac cacacccgac taattttcgt |
| 22981 | atttttagta gagacggggt ttctccatgt tggccaggct gttcttgaac tcctgacctc |
| 23041 | aagtgctcca cctgcgttgg cttgccaaag tgctgggata caggagtgag ccactgcgcc |
| 23101 | tggctgatcc cagcactttt caaatgatgc cgctcaaagc cgtgacttgg cctactttga |
| 23161 | acagcaaact tgttggtgct gttgtcaacc tgaaggcctc tcaaatgcca gcttcaagca |
| 23221 | gggtgtgaat tggccagtgt cagatctcag gagtcctgtg ttgagagtgt ggctttcagc |
| 23281 | tgcggggagc tgcacttggt ggggaaagcc aggcaggtca ccctcacagc cagataatgt |
| 23341 | ggaggtcaga acccaaggaa gggagtgaga cgtccactcc cagtggggga cctggccacc |
| 23401 | catccttggg gacctgagaa agcgtacttc accttggggt gaaggctggg tggggccaga |
| 23461 | gggaccagtg ccctccttag tgcttagggg cagagccacc tgcagcaatg gtatctgcat |
| 23521 | attagcccct ctccaccttc tttctcccgc tgaatcattt ccctcaaagc ccaagagctg |
| 23581 | tcactgcttc tttctccctg ggaagaatgc gtggactctg cctggtgata gactgaagcc |
| 23641 | agaacagtgc cacaccctcg ccttaattcc ttgctaggtg ttctcagatt tatgagactt |
| 23701 | cttagtcaaa tatgagggag gttggatgtg gtggcttgtg cctgtaatcc cagcattttg |
| 23761 | ggaagccgag gtgggaggat cccttgaagc caggagtttg agacaagcct gggcaagaaa |
| 23821 | gcaaaaccct atctctaaaa aaaaaaaaaa aaaaaaaaaa aaaaaaaaaa aaaaaaaatc |
| 23881 | taggagatgc tctttaccct gcctggcctc aaactattaa tagcttcctt tgagcaacat |
| 23941 | tatttattat gaactttcaa acacaaaaaa gtagagagag tagaataaca aatccccatg |
| 24001 | agcccatcac ccaacttcag taattatcaa ttcatggcca tcttgttcac ccctgcctgc |
| 24061 | ttccctgctt cccctcattc tgcagaggtt cttttctttt gagacagagt gttgctctgt |
| 24121 | tgcccaggct ggagtgcagt ggtgcaactt cggctcactg caacctccgc ctcccaggtt |
| 24181 | caagtgattc tcctgcttca gcctctcaag tagctgggat tacagatgcc cgccaccaca |
| 24241 | cctggctaat tttcgtattt ttgttagaga tggggtttca ccatgttggc caggctggtc |
| 24301 | tcgaactcct gacctcaagt gatccgcccg ccttggcctc ccaaaatgct gggattacag |
| 24361 | gtgtgaacca cggtgcctgg ccactgtaca ggttatttat agaagttgga gagtgaaggg |
| 24421 | ttgagaaagc caaggggcag atgcgggtct ggaggatttt gtgcctaagg ccctctcttt |
| 24481 | gctcccagac agcatgaagt aacaatgagg catccacctc ttggttttgt ggcctctgtg |
| 24541 | gatgacgtct ctcaccttga accagttcag agttggagta gcgcaggatc ctgtcttcag |
| 24601 | aggaggggcc gaagcgggtt cctctgttgt caagctcttt ggaggtgcct ggctgctact |
| 24661 | actgtcccag agaggtgatg atgaatgatg ggtgtgtcca gtggcagttt gcccaactga |
| 24721 | ggcaagggct tccactaggc cctgacagag cccttccagc aggcagaaat ccctgtgcta |
| 24781 | ggcaagattc aaactccgta gcatgtctcc tgctcccatc tcttaggaat ggagtccttc |
| 24841 | aggccttgag tcccacattt tccatgatgc tccattaagc agctgatagc accccaacct |
| 24901 | ccagggaaag tgagttcaga gtccttggtc taatgcatct gtgttgaaat tgaggccttc |
| 24961 | ccctgtgttc acctttctgc tctttttctt ttagcccaag gctatgaagg cctcattcgg |
| 25021 | tgctgggcat ggtcactcct agcattcctc actctgttgc taacagcaac agcaataata |
| 25081 | ataagggtta caacttactc cataccttac tgtctgccag gcattaagct aagtgcttta |
| 25141 | catatattaa gtcatttaat cctcataatg accctatgaa agagatacca tctcaaccca |
| 25201 | attgacagct gatttgcaag attaggaggg atgaaggacc caggggacaa tgcgagggaa |
| 25261 | aactctgacc ccggggcccc aggctggatg ttctttatgc ctgtgaacca cagcttatca |
| 25321 | catgtctgga gttagggacc ccacttaaag tgagattttg gctggaggtg gtggatcata |
| 25381 | cctataatcc cagcactttg ggagaccaag gcagaaggac tgcttgaggc caggagttca |
| 25441 | aaaccagtgt aggtaacagc tagaccctat ctctacaaaa aatttaaaaa ttagctgggt |
| 25501 | gtggtggtat gtgcctcaag ttccagctac tcaggaggct gaggtgagag gatcacttga |
| 25561 | gcacaggagt ttgaagttac agtgagctat gatggcacca ctgcacttca gcctaggcaa |
| 25621 | cagagggaga ccctgtcttt aaagtacata gaggtttttc acaccaacac atctctgccc |
| 25681 | agtgtgccaa catctgccac ctactataat agtactataa cactcaatat gtaattaatg |
| 25741 | tagtctcagg gatgttatga caatatgatt acaactatca cgtgtgtgcc cagccaggct |
| 25801 | caatgcccca ggctgggcga ggtggggcag gggacacagc ctaaaatgcc aggcctcagg |
| 25861 | aagccatttg gtttagcaga cattgtttat taaaggagtt acctatgcca gatcgaaggc |
| 25921 | ctaagatgat taagacacta tgagtgcctt caagtggttg gggacgttca tgattgtggt |
| 25981 | acagacaaat aggctttcac atcattcttt tatgtaatca tacaacagat atttgcacct |
| 26041 | acatgtgcag agcactgtga taggcctcag tgacacagaa taatacggca aagaccccac |
| 26101 | ccgatgagcc ccctcccacc acccaccagt acagtagggg gtggtttaat ggagtgttcc |
| 26161 | tggaatatga agtgggggca ggcattaggg gtggcaaagg gacaagtgtt tatctgatca |
| 26221 | gttatgtact gtttataata agtaaatcag cagaggggga ataatactta gaacctatag |
| 26281 | agagtaaatc tgacaagatg aaatgctgat gaaaatatgg aggaaatgaa actctcatgg |
| 26341 | gttttgcagg gaatctaagt cagtgctgtg ttgtgaatgt aggtgtaccc tttgaattca |
| 26401 | tatgttgaat cctaaccccc aaagcaatgg cattaagagg tggggccttt ggggctgggt |
| 26461 | atggtggctc atgactgtaa taccagcact ttgggatgct ggcagggggc agatcacttg |
| 26521 | aagccaggag tctgagatca gcctggccaa catggtgaaa ccccatctgt actaaaaata |
| 26581 | caaaaattag ccaggtgtga tggcgtacat ctgtaatttc agccactcgg gaggctgaga |
| 26641 | caggagaata gcttgaaccc agtaggtgga gatttcagtg agccgagatc gtgccactgc |
| 26701 | actccagcct gggtgacaga gcgagactcc atctcaaaaa aataataaag atgtggggcc |
| 26761 | tgtgggaggt ggttaggtca tgagggtgga gatcatgaat ggggttagca ccttataaaa |
| 26821 | caggcttgag ggagcccttc tgtcccttct accatgtgtg gatgcagtga gaaggcaccg |
| 26881 | tatctctgaa gcagagagcc cgccctggac actggatctg ctggcacctt gatcttggac |
| 26941 | ttcccagcct ctagaactgt gagaaataat tttttgttgt ttacaaatta cccaggctaa |
| 27001 | ggtgtttcat tgtaacctga atggaccaag ctggtgtgac cctgttggaa aactggcagt |
| 27061 | atctaccaaa agccgaacat acgtataaac tgatccagca gttccactcc tgggtatgta |
| 27121 | caccacagaa agctatgtcc accgagacat tggcaagaat gtttctaacc acacgctgac |
| 27181 | tgtagcccca aacctgaaac aacccaaatg tccatccacc aacccaaatg tccatccaca |
| 27241 | gttgaagcta cagtgaagtc acagggtcga atagtactgc acagcaacga atatgaatga |
| 27301 | aaatatcgct atgcacagca acatggataa atttcacaga catgaggtca agcaaaagag |
| 27361 | gtcagagtcc tcatcatcaa gagagaattc attgtatgat tctcttccta caaaaagtac |
| 27421 | agaaataagc aaaactgatc catggtgtta gaagccaggg gaacagttaa caggggaggg |
| 27481 | atactgggga ggggcatcct ggagtgctgg tctacctcat ctgggtgttg atttcacgag |
| 27541 | tattgtcagt ttgtttccag actccctgtt ggagatgtgg aaataaaaac cacctaaaca |
| 27601 | agagcagaga ggccatttgg tcaaagtttg caaaggagtc agccatgatt gcttgtattt |
| 27661 | ggcaggggtc aaaggcaggc agggactgtg aaatgttata gtggaaaaaa agggaaggct |
| 27721 | ctgggtgtgc tgtgattgga gattgttggc atggggacag agcggactaa ctggaggggc |
| 27781 | atctttggtt ggttgggggg gtatatttgg ctttctctgg ttggtctgga gttggaagag |
| 27841 | ggggtgtggt ggctggggat tgggaagaag ctggcagcca ctaagttcag actgttctgg |
| 27901 | gtccgattgc tgctgaggct gtggtttggc ttccttggct tcccaggctg gtcatgggtt |
| 27961 | tctggccaga gtctattgtc atatgtggcc tggccattgt ccagttgtat gttcagtctc |
| 28021 | ttggaaggaa gggtattgac tctgagaggg gccaccatcg ctggaatggg ggacacacag |
| 28081 | tacttcctcc agctgcctac acccccctag ggtcagtggc gcctgcctgt gagggtgagc |
| 28141 | ccaatggcta gagggctctg ctccaagtca ttgcttacta cacccacaaa cattcttcgt |
| 28201 | tctttaaggc ctaacttaaa gcccagatcc tacaggaaac cttgattaga cccctctctt |
| 28261 | tattaagctt cctaagatca aaccctgctt ttgtgtaaat gctgacctcc ttgcctacat |
| 28321 | tttaaaaacc tagagctggg catgatggcc ccagcctgta atcccagtga ttcaggagac |
| 28381 | tgaggtggga ggattgctag aagccaggag ttcgagacca gcctgggtaa catagctaga |
| 28441 | ccacatctct taaaataaaa tagttaattt agccaggcat gatgatatat gcctgtagtc |
| 28501 | ccaactactt ggaaggctga ggtgtgagga tctttgagcc cgggaggtcg aggctacagt |
| 28561 | aagctatgat ctcaccactg tactccagcc tgggtgacag agcgagaccc agactcaaaa |
| 28621 | aataaaaata aaaaccctga atatcttcct tctacttctt cagtgctgtt tttatttaaa |
| 28681 | aaaaaaaaaa accagccaaa accacaactt tttactgaag tgtaatgtaa atgctgtaaa |
| 28741 | aggcagtgaa aggcacaagg gaggtggagg ggtaggaagg gtggaagtgg cgggaggaag |
| 28801 | tggcagggca ggcaaaatga agggaagccc tgggttcttg tcctgcatcc gcagccagct |
| 28861 | cccactttcc tcaccctcca ggacctgtaa actgtgaggc tggaccagtt atgtcaaatc |
| 28921 | tgtcctcccc cagagctcag tccctctgcc cttgggtgtc cttggcacaa ggcaggctag |
| 28981 | gctgcaccag cttcctccat ctccgtcctg cctcccccat ccccaggtgc cattcccaca |
| 29041 | ccatctgaat cactgatttc ctcgcaatca gacgctatct tccagttaat cacttcgctt |
| 29101 | gtatttaaca taagaaagaa aaaccctttc attatcacat acagctggaa atcggcttct |
| 29161 | tgcaggaggc gtatccaaag gaattggaga agagataaac tggtaattgg tgaaagaatt |
| 29221 | actttaattt tttttcctac ttgctgtcat gatgatgtcc ttagaattgt gagcccgtgg |
| 29281 | acacttctgt acaataaatc tgctattatt acttctagaa ctaca |

STING: By “STING,” “TMEM173,” “stimulator of interferon genes,” and the like are meant a polynucleotide encoding a STING polypeptide or fragment thereof (e.g., a human STING) e.g., a polynucleotide encoding the amino acid sequence of NCBI Accession No. Q86WV6.1. An exemplary amino acid sequence is provided at NCBI Accession No. Q86WV6:

| 1 | mphsslhpsi | pcprghgaqk | aalvllsacl | vtlwglgepp | ehtlrylvlh | laslqlglll |
|---|---|---|---|---|---|---|
| 61 | ngvcslaeel | rhihsryrgs | ywrtvraclg | cplrrgalll | lsiyfyyslp | navgppftwm |
| 121 | lallglsqal | nillglkgla | paeisavcek | gnfnvahgla | wsyyigylrl | ilpelqarir |
| 181 | tynqhynnll | rgavsqrlyi | llpldcgvpd | nlsmadpnir | fldklpqqtg | dhagikdrvy |
| 241 | snsiyellen | gqragtcvle | yatplqtlfa | msqysqagfs | redrleqakl | fortledila |
| 301 | dapesqnner | liayqepadd | ssfslsqevl | rhlrqeekee | vtvgslktsa | vpststmsqe |
| 361 | pellisgmek | plplrtdfs | | | | |
| An exemplary nucleotide sequence is provided at NCBI Accession No. NM 198282.3: | | | | | | |
| 1 | tataaaaata | gctcttgtta | ccggaaataa | ctgttcattt | ttcactcctc | cctcctaggt |
| 61 | cacacttttc | agaaaaagaa | tctgcatcct | ggaaaccaga | agaaaaatat | gagacgggga |
| 121 | atcatcgtgt | gatgtgtgtg | ctgcctttgg | ctgagtgtgt | ggagtcctgc | tcaggtgtta |
| 181 | ggtacagtgt | gtttgatcgt | ggtggcttga | ggggaacccg | ctgttcagag | ctgtgactgc |
| 241 | ggctgcactc | agagaagctg | cccttggctg | ctcgtagcgc | cgggccttct | ctcctcgtca |
| 301 | tcatccagag | cagccagtgt | ccgggaggca | gaagatgccc | cactccagcc | tgcatccatc |
| 361 | catcccgtgt | cccaggggtc | acggggccca | gaaggcagcc | ttggttctgc | tgagtgcctg |
| 421 | cctggtgacc | ctttgggggc | taggagagcc | accagagcac | actctccggt | acctggtgct |
| 481 | ccacctagcc | tccctgcagc | tgggactgct | gttaaacggg | gtctgcagcc | tggctgagga |
| 541 | gctgcgccac | atccactcca | ggtaccgggg | cagctactgg | aggactgtgc | gggcctgcct |
| 601 | gggctgcccc | ctccgccgtg | gggccctgtt | gctgctgtcc | atctatttct | actactccct |
| 661 | cccaaatgcg | gtcggcccgc | ccttcacttg | gatgcttgcc | ctcctgggcc | tctcgcaggc |
| 721 | actgaacatc | ctcctgggcc | tcaagggcct | ggccccagct | gagatctctg | cagtgtgtga |
| 781 | aaaagggaat | ttcaacgtgg | cccatgggct | ggcatggtca | tattacatcg | gatatctgcg |
| 841 | gctgatcctg | ccagagctcc | aggcccggat | togaacttac | aatcagcatt | acaacaacct |
| 901 | gctacggggt | gcagtgagcc | agcggctgta | tattctcctc | ccattggact | gtggggtgcc |
| 961 | tgataacctg | agtatggctg | accccaacat | tcgcttcctg | gataaactgc | cccagcagac |
| 1021 | cggtgaccat | gctggcatca | aggatcgggt | ttacagcaac | agcatctatg | agcttctgga |
| 1081 | gaacgggcag | cgggcgggca | cctgtgtcct | ggagtacgcc | acccccttgc | agactttgtt |
| 1141 | tgccatgtca | caatacagtc | aagctggctt | tagccgggag | gataggcttg | agcaggccaa |
| 1201 | actcttctgc | cggacacttg | aggacatcct | ggcagatgcc | cctgagtctc | agaacaactg |
| 1261 | ccgcctcatt | gcctaccagg | aacctgcaga | tgacagcagc | ttctcgctgt | cccaggaggt |
| 1321 | tctccggcac | ctgcggcagg | aggaaaagga | agaggttact | gtgggcagct | tgaagacctc |
| 1381 | agcggtgccc | agtacctcca | cgatgtccca | agagcctgag | ctcctcatca | gtggaatgga |
| 1441 | aaagcccctc | cctctccgca | cggatttctc | ttgagaccca | gggtcaccag | gccagagcct |
| 1501 | ccagtggtct | ccaagcctct | ggactggggg | ctctcttcag | tggctgaatg | tccagcagag |
| 1561 | ctatttcctt | ccacaggggg | ccttgcaggg | aagggtccag | gacttgacat | cttaagatgc |
| 1621 | gtcttgtccc | cttgggccag | tcatttcccc | tctctgagcc | tcggtgtctt | caacctgtga |
| 1681 | aatgggatca | taatcactgc | cttacctccc | tcacggttgt | tgtgaggact | gagtgtgtgg |
| 1741 | aagtttttca | taaactttgg | atgctagtgt | acttaggggg | tgtgccaggt | gtctttcatg |
| 1801 | gggccttcca | gacccactcc | ccacccttct | ccccttcctt | tgcccgggga | cgccgaactc |
| 1861 | tctcaatggt | atcaacaggc | tccttcgccc | tctggctcct | ggtcatgttc | cattattggg |
| 1921 | gagccccagc | agaagaatgg | agaggaggag | gaggctgagt | ttggggtatt | gaatcccccg |
| 1981 | gctcccaccc | tgcagcatca | aggttgctat | ggactctcct | gccgggcaac | tcttgcgtaa |
| 2041 | tcatgactat | ctctaggatt | ctggcaccac | ttccttccct | ggccccttaa | gcctagctgt |
| 2101 | gtatcggcac | ccccacccca | ctagagtact | ccctctcact | tgcggtttcc | ttatactcca |
| 2161 | cccctttctc | aacggtcctt | ttttaaagca | catctcagat | tacccaaaaa | aaaaaaaaaa |
| 2221 | aaa | | | | | |

In certain embodiments, the compositions embodied herein comprising a stimulator of interferon genes (STING) molecule modulates expression, function or activity of one or more innate immune response genes and/or STING-dependent genes comprising IFN, TREX1, CXCL11, IFITI, SNPH, DDX58, CUL4A, HERC5, IFIT, IFIT3, PMAIP1, OASL, CH25H, NFLBIZ, RSAD2, GBP4, IFNB1, ZC3HAV1, CCL5, ATF3, KLF4, ZFP36L2, ARL4A, PTGER4, OASLI, LOC667370, IFIT2, CXCL10, HMGA1, CCL4, GBP2, SAMD9L, COX7A2L, CCK, NNMT, TYK1, MX2, CD274, IFI205, CXCL9, LIGP2, IGTP, USP18, LOC100048346, CCL7, 1133, GBP3, OASL2, IRFI, GBP1, MT-ND4L OR TAFID.

TRIF (TIR-Domain Containing Adaptor-Inducing Interferon-β)

By “TRIF,” “TIR-domain containing adaptor-inducing interferon-β,” and the like are meant a polynucleotide encoding a TRIF polypeptide or fragment thereof, e.g., a human TRIF (e.g., a polynucleotide encoding the amino acid sequence of NCBI Accession No. BAC44839.1 or AB093555.1 or NP_891549.1). An exemplary amino acid sequence is provided at NCBI Accession No. BAC44839.1:

| 1 | mactgpslps | afdilgaagq | dkllylkhkl | ktprpgcqgq | dllhamvllk | lgqetearis |
|---|---|---|---|---|---|---|
| 61 | lealkadava | rlvarqwagv | dstedpeepp | dvswavarly | hllaeeklcp | aslrdvayqe |
| 121 | avrtlssrdd | hrlgelqdea | rnrcgwdiag | dpgsirtlqs | nlgclppssa | lpsgtrslpr |
| 181 | pidgvsdwsq | gcslrstgsp | aslasnleis | qsptmpflsl | hrsphgpskl | cddpqaslvp |
| 241 | epvpggcqep | eemswppsge | iasppelpss | pppglpevap | datstglpdt | paapetstny |
| 301 | pvectegsag | pqslplpile | pvknpcsvkd | qtplqlsved | ttspntkpcp | ptpttpetsp |
| 361 | pppppppsst | pcsahltpss | lfpsslesss | eqkfynfvil | haradehial | rvreklealg |
| 421 | vpdgatfced | fqvpgrgels | clqdaidhsa | fiillltsnf | dcrlslhqvn | qammsnltrq |
| 481 | gspdcvipfl | plesspaqls | sdtasllsgl | vrldehsqif | arkvantfkp | hrlqarkamw |
| 541 | rkeqdtralr | eqsqhldger | mqaaalnaay | saylqsylsy | qaqmeqlqva | fgshmsfgtg |
| 601 | apygarmpfg | gqvplgappp | fptwpgcpqp | pplhawqagt | ppppspqpaa | fpqslpfpqs |
| 661 | pafptaspap | pqspglqpli | ihhaqmvqlg | lnnhmwnqrg | sqapedktqe | ae |
| An exemplary nucleic acid sequence is provided at NCBI Accession No. NM 182919.3: | | | | | | |
| 1 | ttcccagggc | gcgggccgcg | gggtggagcc | agcgccctca | gegcgctacg | gtccgcgggc |
| 61 | aactccgcag | aagccccagc | ccccaggacc | ccaggaccca | gtggcgcagc | cggcagcccc |
| 121 | ggatccctga | tctgcttggg | cagctcctgc | agaacctgga | acagtgaatg | ggtaggggac |
| 181 | actgggcgtg | cagaaggcgg | ggggcagtgt | ggaacatgcc | ttcaccacct | ccagcttctg |
| 241 | ctgccggagg | ctgcacccac | ctgtgcccat | ggcctgcaca | ggcccatcac | ttcctagcgc |
| 301 | cttcgacatt | ctaggtgcag | caggccagga | caagctcttg | tatctgaagc | acaaactgaa |
| 361 | gaccccacgc | ccaggctgcc | aggggcagga | cctcctgcat | gccatggttc | tcctgaagct |
| 421 | gggccaggaa | actgaggcca | ggatctctct | agaggcattg | aaggccgatg | cggtggcccg |
| 481 | gctggtggcc | cgccagtggg | ctggcgtgga | cagcaccgag | gacccagagg | agcccccaga |
| 541 | tgtgtcctgg | gctgtggccc | gcttgtacca | cctgctggct | gaggagaagc | tgtgccccgc |
| 601 | ctcgctgcgg | gacgtggcct | accaggaagc | cgtccgcacc | ctcagctcca | gggacgacca |
| 661 | ccggctgggg | gaacttcagg | atgaggcccg | aaaccggtgt | gggtgggaca | ttgctgggga |
| 721 | tccagggagc | atccggacgc | tccagtccaa | tctgggctgc | ctcccaccat | cctcggcttt |
| 781 | gccctctggg | accaggagcc | tcccacgccc | cattgacggt | gtttcggact | ggagccaagg |
| 841 | gtgctccctg | cgatccactg | gcagccctgc | ctccctggcc | agcaacttgg | aaatcagcca |
| 901 | gtcccctacc | atgcccttcc | tcagcctgca | ccgcagccca | catgggccca | gcaagctctg |
| 961 | tgacgacccc | caggccagct | tggtgcccga | gcctgtcccc | ggtggctgcc | aggagcctga |
| 1021 | ggagatgagc | tggccgccat | cgggggagat | tgccagccca | ccagagctgc | caagcagccc |
| 1081 | acctcctggg | cttcccgaag | tggccccaga | tgcaacctcc | actggcctcc | ctgatacccc |
| 1141 | cgcagctcca | gaaaccagca | ccaactaccc | agtggagtgc | accgaggggt | ctgcaggccc |
| 1201 | ccagtctctc | cccttgccta | ttctggagcc | ggtcaaaaac | ccctgctctg | tcaaagacca |
| 1261 | gacgccactc | caactttctg | tagaagatac | cacctctcca | aataccaagc | cgtgcccacc |
| 1321 | tactcccacc | accccagaaa | catcccctcc | tcctcctcct | cctcctcctt | catctactcc |
| 1381 | ttgttcagct | cacctgaccc | cctcctccct | gttcccttcc | tccctggaat | catcatcgga |
| 1441 | acagaaattc | tataactttg | tgatcctcca | cgccagggca | gacgaacaca | tcgccctgcg |
| 1501 | ggttcgggag | aagctggagg | cccttggcgt | gcccgacggg | gccaccttct | gcgaggattt |
| 1561 | ccaggtgccg | gggcgcgggg | agctgagctg | cctgcaggac | gccatagacc | actcagcttt |
| 1621 | catcatccta | cttctcacct | ccaacttcga | ctgtcgcctg | agcctgcacc | aggtgaacca |
| 1681 | agccatgatg | agcaacctca | cgcgacaggg | gtcgccagac | tgtgtcatcc | ccttcctgcc |
| 1741 | cctggagagc | tccccggccc | agctcagctc | cgacacggcc | agcctgctct | ccgggctggt |
| 1801 | gcggctggac | gaacactccc | agatcttcgc | caggaaggtg | gccaacacct | tcaagcccca |
| 1861 | caggcttcag | gcccgaaagg | ccatgtggag | gaaggaacag | gacacccgag | ccctgcggga |
| 1921 | acagagccaa | cacctggacg | gtgagcggat | gcaggcggcg | gcactgaacg | cagcctactc |
| 1981 | agcctacctc | cagagctact | tgtcctacca | ggcacagatg | gagcagctcc | aggtggcttt |
| 2041 | tgggagccac | atgtcatttg | ggactggggc | gccctatggg | gctcgaatgc | cctttggggg |
| 2101 | ccaggtgccc | ctgggagccc | cgccaccctt | tcccacttgg | ccggggtgcc | cgcagccgcc |
| 2161 | acccctgcac | gcatggcagg | ctggcacccc | cccaccgccc | tccccacage | cagcagcctt |
| 2221 | tccacagtca | ctgcccttcc | cgcagtcccc | agccttccct | acggcctcac | ccgcaccccc |
| 2281 | tcagagccca | gggctgcaac | ccctcattat | ccaccacgca | cagatggtac | agctggggct |
| 2341 | gaacaaccac | atgtggaacc | agagagggtc | ccaggcgccc | gaggacaaga | cgcaggaggc |
| 2401 | agaatgaccg | cgtgtccttg | cctgaccacc | tggggaacac | ccctggaccc | aggcatcggc |
| 2461 | caggacccca | tagagcaccc | cggtctgccc | tgtgccctgt | ggacagtgga | agatgaggtc |
| 2521 | atctgccact | ttcaggacat | tgtccgggag | cccttcattt | aggacaaaac | gggcgcgatg |
| 2581 | atgccctggc | tttcagggtg | gtcagaactg | gatacggtgt | ttacaattcc | aatctctcta |
| 2641 | tttctgggtg | aagggtcttg | gtggtggggg | tattgctacg | gtcttttaat | tataataaat |
| 2701 | atttattgaa | tgcttccgca | gcaaaaa | | | |

pLxIS Motif The adaptor proteins (i.e., MAVS, STING and TRIF) are phosphorylated in response to stimulation at their respective C-terminal consensus motif-pLxIS (wherein p: hydrophilic residue, x: any residue, S, Phosphorylation site). Phosphorylation of the serine residue recruits IRF3 to the active adaptor protein and is required for IRF3 activation (Liu et al., Science 347: 6227, 2015). Research has shown that fragments of the adaptors containing the charged pLxIS motif appeared to be sufficient for activity.

In aspects, the motif (e.g., pLxIS motif) is a polypeptide fragment of the MAVS protein (e.g., human MAVS protein), STING protein (e.g., human STING protein), or the TRIF protein (e.g. human TRIF protein). The pLxIS motif described herein is found in adaptor proteins MAVS, STING and TRIF, each that activate the downstream protein kinase TBK1, which in turn phosphorylates the transcription factor interferon regulatory factor IRF3 (IRF3). The phosphorylation of IRF3 drives type I IFN production. In aspects, any portion or fragment of the pLxIS motif may be contemplated.

By way of example, the amino acid sequence of the pLxIS motif comprises residues sequence:

(SEQ ID NO: 1)
VTMNAPMTSVAPPPSVLSQEPRLLISGMDQPLPLRTDLI

### Pharmaceutical Compositions

In certain embodiments, the present invention provides for a pharmaceutical composition comprising a synthetic gene as identified herein. The composition can be suitably formulated and introduced into a subject or the environment of a cell (e.g., a neoplasia, a cancer cell or a tumor) by any means recognized for such delivery.

Such compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

The composition may be administered directly into the cancerous tumor, or in some embodiments can be administered to the immune cell.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

As defined herein, a therapeutically effective amount of an adjuvant-containing composition of the invention targeting a disease or disorder (i.e., an effective dosage) depends on the immunogen and target disease or disorder selected. For instance, single dose amounts of an immunogen of an immunogen-adjuvant composition of the invention targeting a disease or disorder in the range of approximately 1 μg to 1000 mg may be administered; in some embodiments, 10, 30, 100, or 1000 μg, or 10, 30, 100, or 1000 ng, or 10, 30, 100, or 1000 μg, or 10, 30, 100, or 1000 mg may be administered. In some embodiments, 1-5 g of the compositions can be administered.

A therapeutically effective amount of the compound of the present invention can be determined by methods known in the art. In addition to depending on the immunogen used, the therapeutically effective quantities of a pharmaceutical composition of the invention will depend on the age and on the general physiological condition of the patient and the route of administration. In certain embodiments, the therapeutic doses will generally be between about 10 and 2000 mg/day and preferably between about 30 and 1500 mg/day. Other ranges may be used, including, for example, 50-500 mg/day, 50-300 mg/day and 100-200 mg/day.

Administration may be a single dose, multiple doses spaced at intervals to allow for an immunogenic response to occur, once a day, twice a day, or more often, and may be decreased during a maintenance phase of a disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of an immunogenic, adjuvant-containing composition targeting a disease, disorder or infectious agent can include a single treatment or, optionally, can include a series of treatments.

### Methods of Treatment

The invention includes methods for treating or preventing cancer with the synthetic genes described herein.

In aspects, the invention describes a composition for manipulating an pathway (e.g., immune pathways, inflammation, cell death pathways, interferon expression, inflammasome pathway, induce or suppress cell division, differentiation, and cell-cell communication, and migration, phagocytosis, and the like), the composition comprises a synthetic gene, and the synthetic gene comprises a motif encoded from a signaling or targeting protein which stimulates a response from the pathway, and is appended to a protein that does not induce the response. The composition may be administered directly into the cancerous tumor, or in some embodiments can be administered to the immune cell.

In aspects, the invention provides methods for manipulating an immune pathway in a cell. In embodiments, the methods involve a composition comprising a synthetic gene including a motif of an adaptor protein (i.e., pLxIS of STING, MAVS, or TRIF) in the cell. In related embodiments, the methods involve contacting the cell with the synthetic gene described herein.

In embodiments, the cell is in a subject. In related embodiments, contacting occurs by therapeutic administration of the inhibitor to the subject in the form of a pharmaceutical composition.

In aspects, the invention provides methods for treating or preventing cancer in a subject. In embodiments, the method involves administering to the subject a composition comprising a synthetic gene including a motif of an adaptor protein (i.e., pLxIS of STING, MAVS, or TRIF) in the cell as described herein.

In any of the above aspects and embodiments, the methods further involve contacting the cell with or administering to the subject an immunotherapeutic agent.

In any of the above aspects and embodiments, the subject is a mammal (e.g., human) or the cell is from a mammal (e.g., human).

Methods for evaluating the therapeutic efficacy of the methods of the invention are standard in the art. For example, efficacy of treatment can be evaluated by assessing viral levels (antigenic levels, RNA levels, and the like), patient symptoms, autoantibody levels, and the like.

### Combination Therapies

The agents and pharmaceutical compositions described herein can also be administered in combination with another therapeutic molecule. The therapeutic molecule can be any compound used to treat viral infection, autoimmune disease, or symptoms thereof. Examples of such compounds include, but are not limited to, anti-viral agents, immunosuppressants, anti-inflammatories, and the like.

The synthetic gene composition can be administered before, during, or after administration of the additional therapeutic agent. In embodiments, the synthetic gene composition is administered before the first administration of the additional therapeutic agent. In embodiments, the synthetic gene composition is administered after the first administration of the additional therapeutic agent (e.g., 1, 2, 3, 4, 5, 6, 7, 8, 9, 10, 11, 12, 13, 14 days or more). In embodiments, synthetic gene composition is administered simultaneously with the first administration of the additional therapeutic agent.

The amount of therapeutic agent administered to a subject can readily be determined by the attending physician or veterinarian. Generally, an efficacious or effective amount of a synthetic gene composition and an additional therapeutic is determined by first administering a low dose of one or both active agents and then incrementally increasing the administered dose or dosages until a desired effect is observed (e.g., reduced symptoms associated with viral infection or autoimmune disease), with minimal or no toxic side effects. Applicable methods for determining an appropriate dose and dosing schedule for administration of a combination of the present invention are described, for example, in Goodman and Gilman's The Pharmacological Basis of Therapeutics, 11th Edition., supra, and in Remington: The Science and Practice of Pharmacy, 20th and 21st Editions, supra.

### Kits

The invention also includes kits that include a composition of the invention, optionally also including a synthetic gene (e.g. a gene that alters the TLR and IL-1R signaling pathways), and instructions for use thereof. The composition can be included in a kit, container, pack, or dispenser together with instructions for administration.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. It is to be understood and expected that variations in the principles of invention herein disclosed may be made by one skilled in the art and it is intended that such modifications are to be included within the scope of the present invention

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself, and, each of these documents or references (“herein-cited references”), as well as each document or reference cited in each of the herein-cited references (including any manufacturer's specifications, instructions, etc.), is hereby expressly incorporated herein by reference. In case of conflict, the present specification, including definitions, will control. In addition, the materials, methods, and examples are illustrative only and not intended to be limiting.

### EXAMPLES

### Example 1: Generation of MYD88 Alleles Containing the pLxIS Motif

On a broad scale, scrutinizing other PRR signaling pathways beyond the TLR pathway, obvious commonalities are identified showing that TBK1 is engaged by the signaling adaptors MAVS and STING which converge at the activation of IRF3 dependent IFN responses. In contrast, MyD88 dependent TBK1 activation does not activate IRF3 or induce IFN.

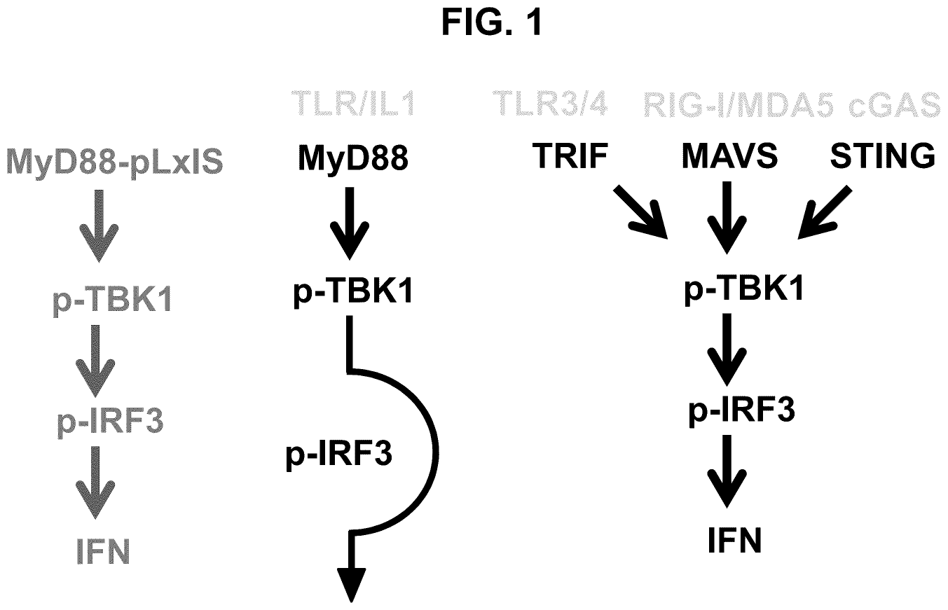

A study showing that TRIF, MAVS, and STING all share a pLxIS motif that serves as a platform to recruit and activate IRF3 was recently reported (Liu, et al., Science 2015) (FIG. 1). MyD88 did not contain a pLxIS motif Accordingly, it was hypothesized that there was selective pressure that eliminated such a motif from MyD88 due to the diverse microbial ligands that induce signaling through this adaptor. Therefore, the addition of the pLxIS motif to MyD88 via a synthetic biology approach was tested to determine whether IRF3 activation and IFN production by the Myddosome was achieved.

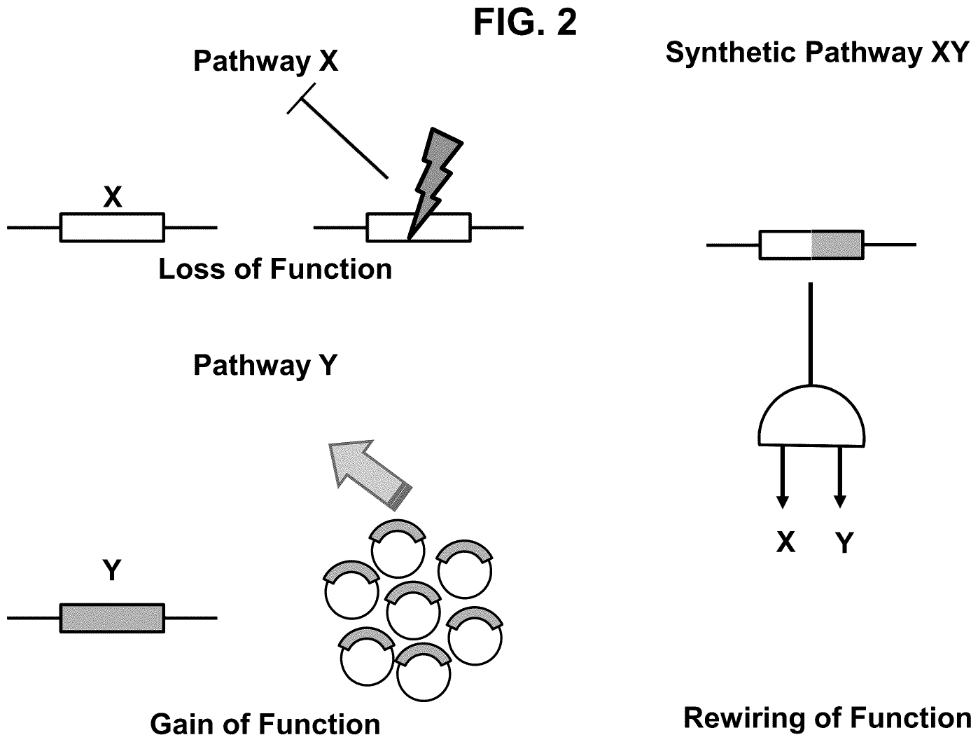

Classic studies of innate immune signaling have greatly benefited from either a loss of function analysis (e.g., chemical mutagenesis, tn, small molecule perturbation, gene knockdown, ko etc, i.e. TLR4, Gasdermin) and gain of function analyses (cDNA overexpression: ie. TLR/STING). Based on the knowledge obtained from these previous approaches, synthetic biology allowed for the rewiring signaling process and created novel signaling circuits (FIG. 2).

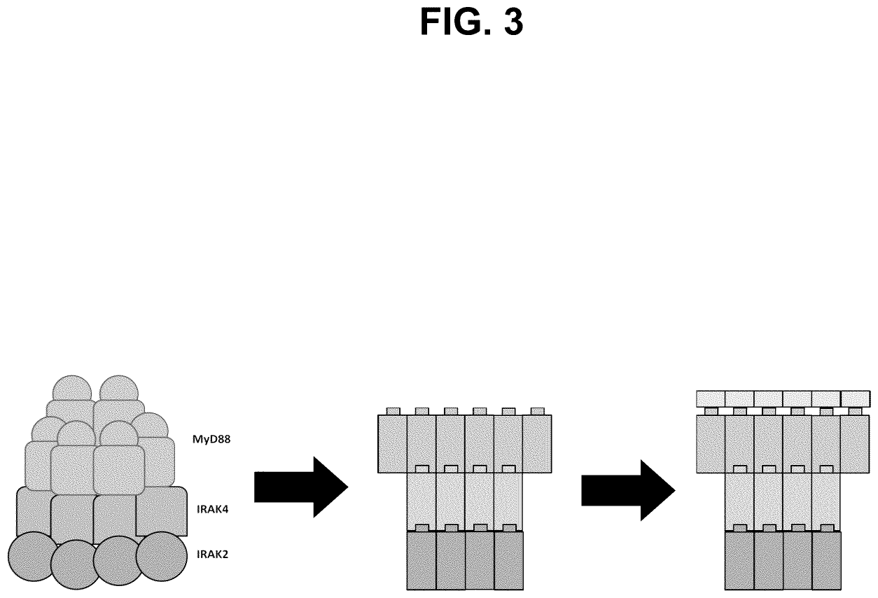

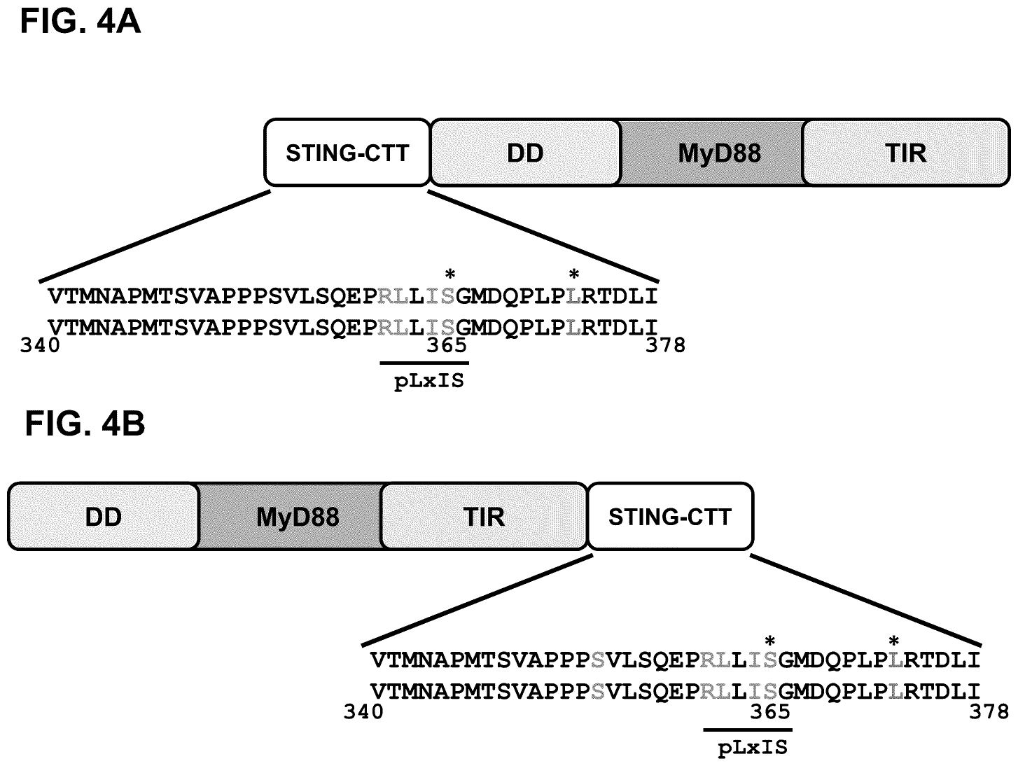

The functionality of signaling proteins can be segregated into small domains and motifs. This modularity enabled the engineering of novel signaling platforms with a unique signal transduction outcome (FIG. 3). The MyD88 allele containing the pLxIS motif was generated as depicted in (FIG. 4). The MyD88 allele contains a death domain (DD), a linker (MyD88) and a TIR domain. The pLxIS motif was added at either the N or C-terminus of the MyD88 gene, either to the DD or the TIR domain. TBK1 phosphorylated STING-CTT at 5365, which further recruited IRF3. L373 was also directly involved in IRF3 recruitment. Neither S365 nor L373 was required for STING-TBK1 interaction.

### Example 2: MyD88-pLxIS Studies

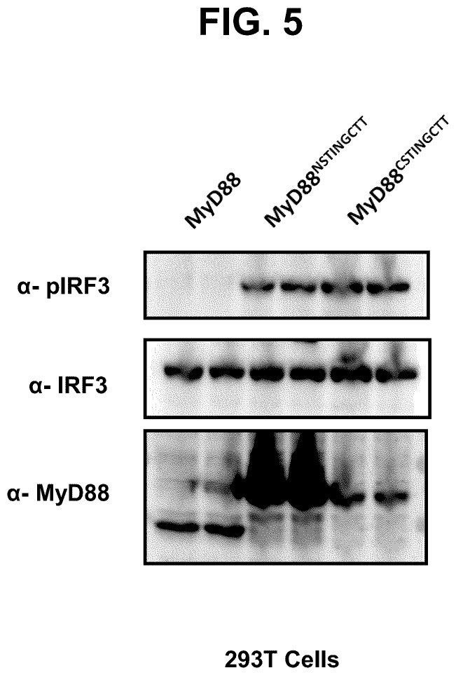

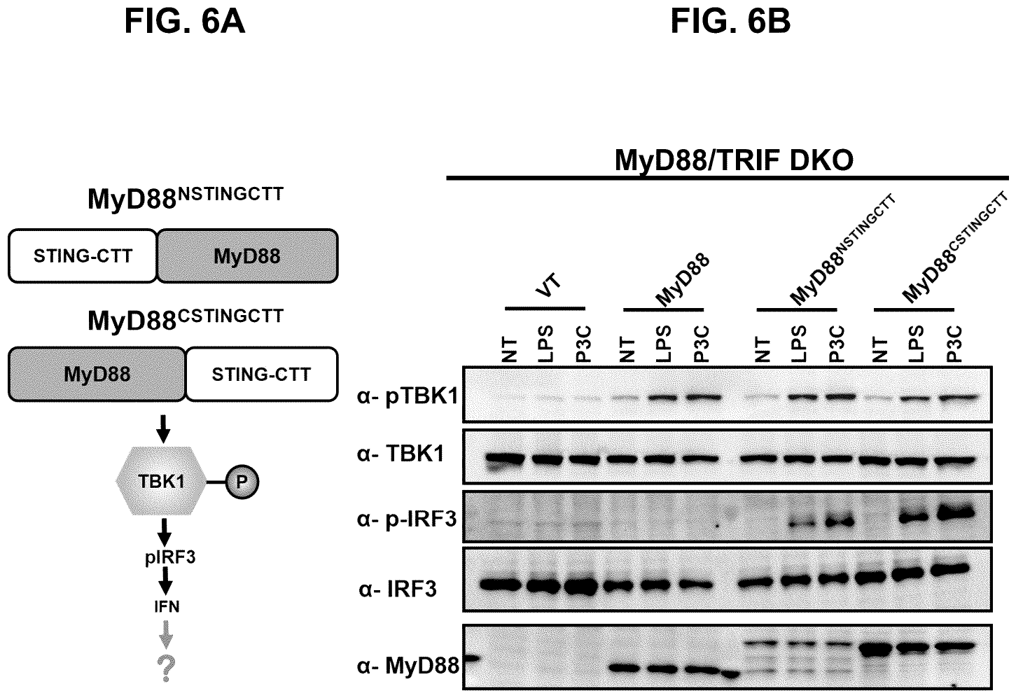

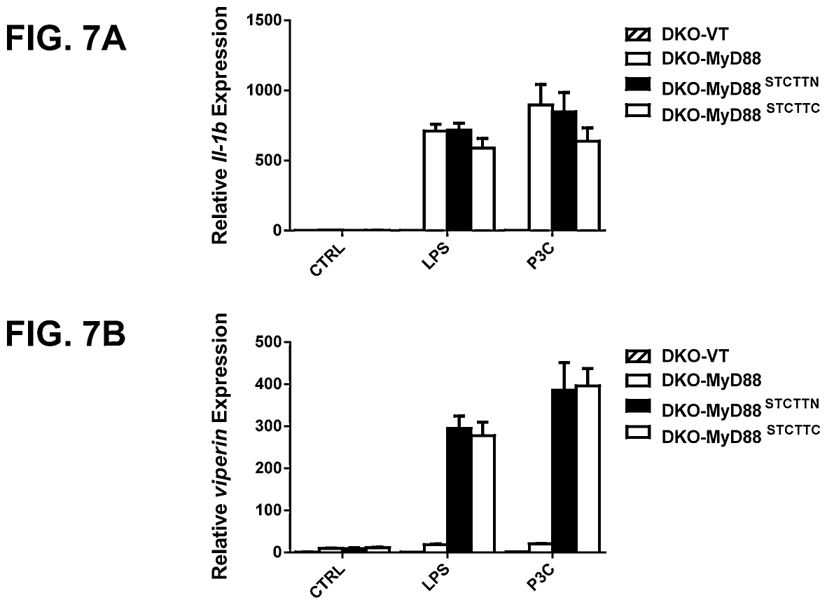

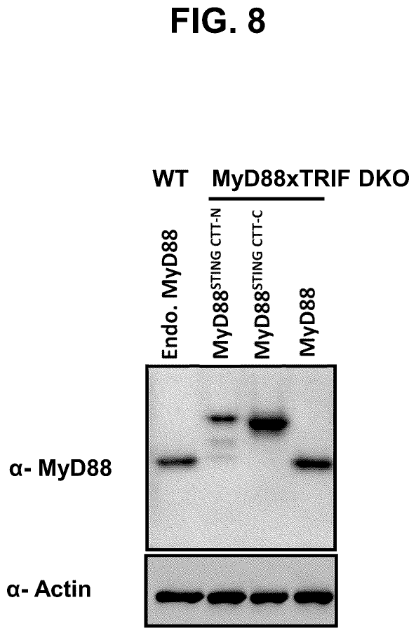

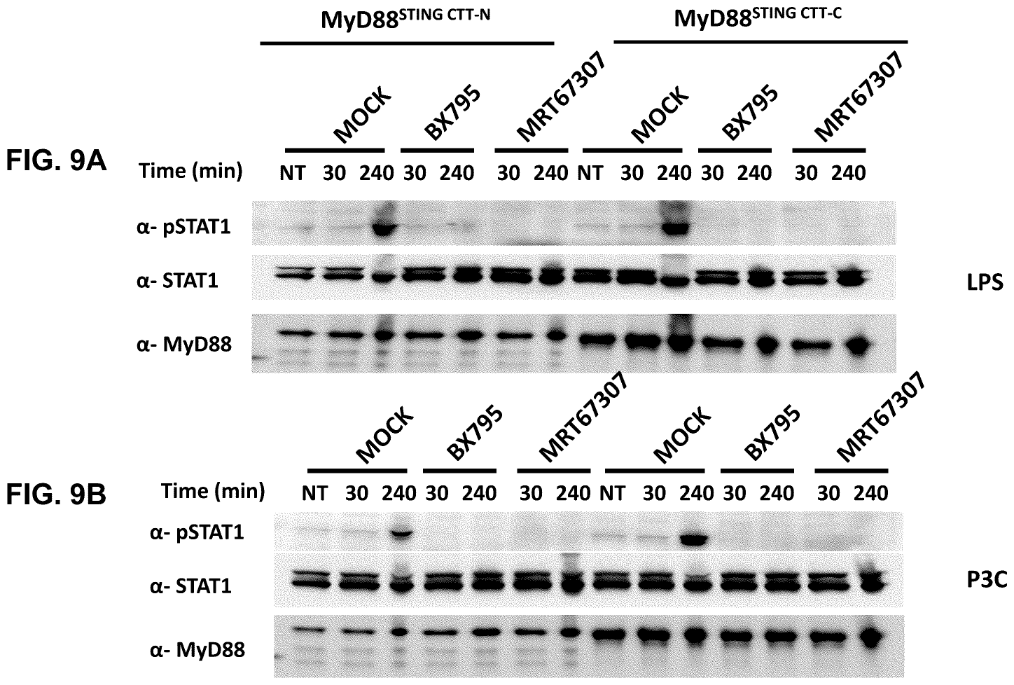

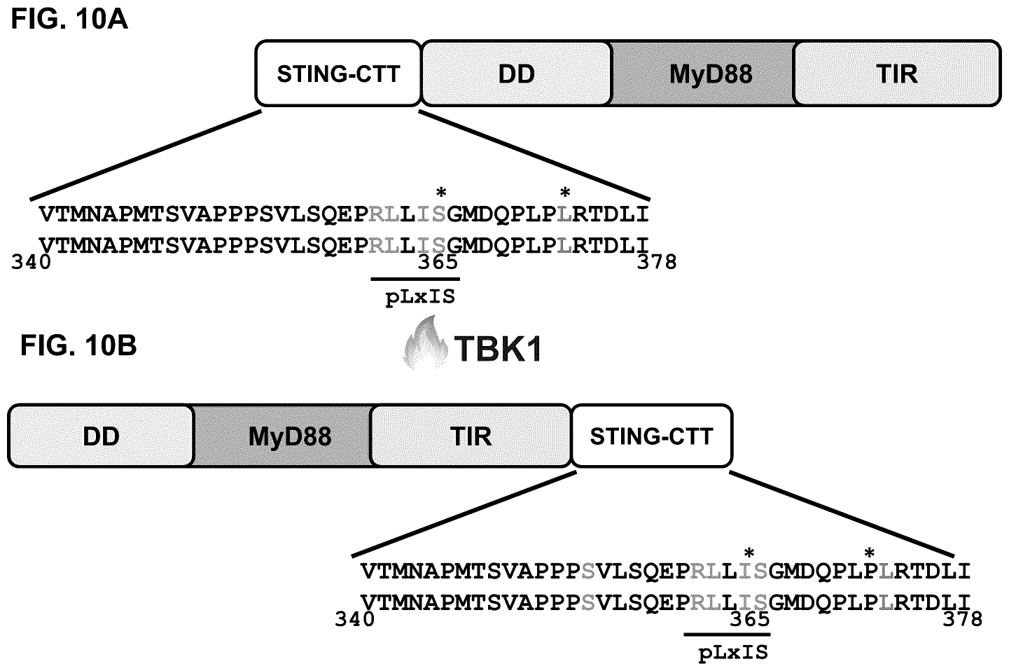

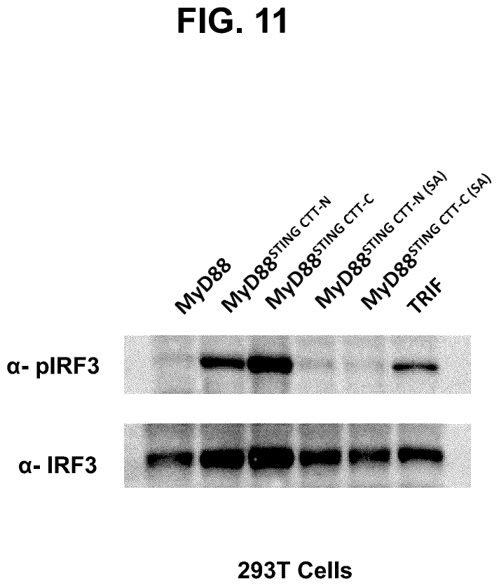

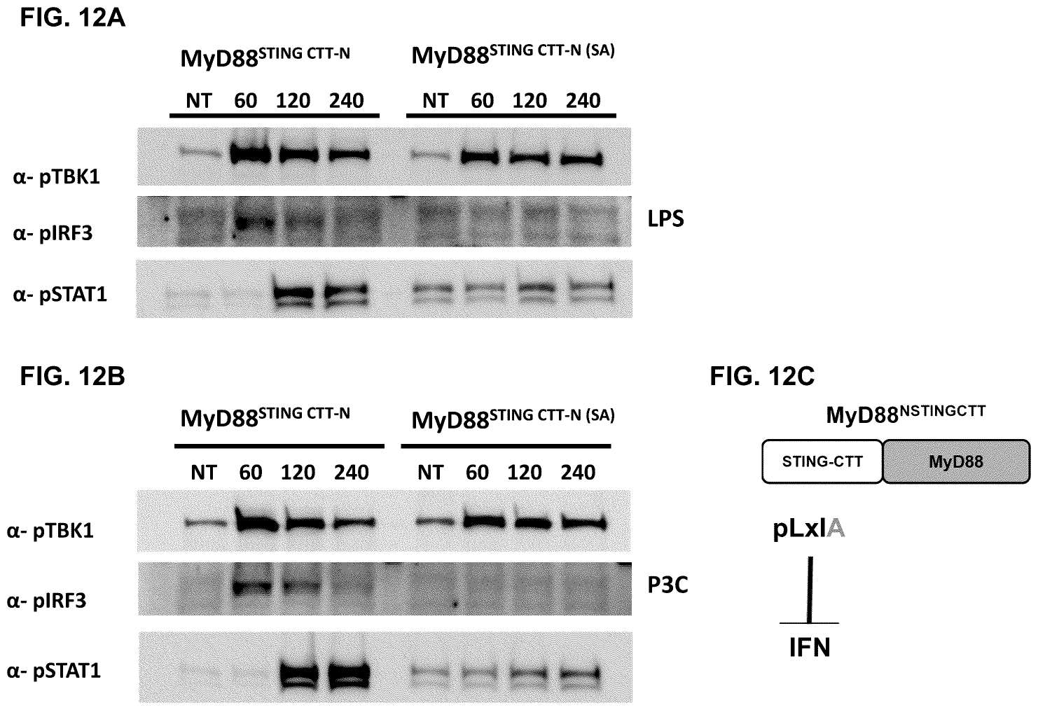

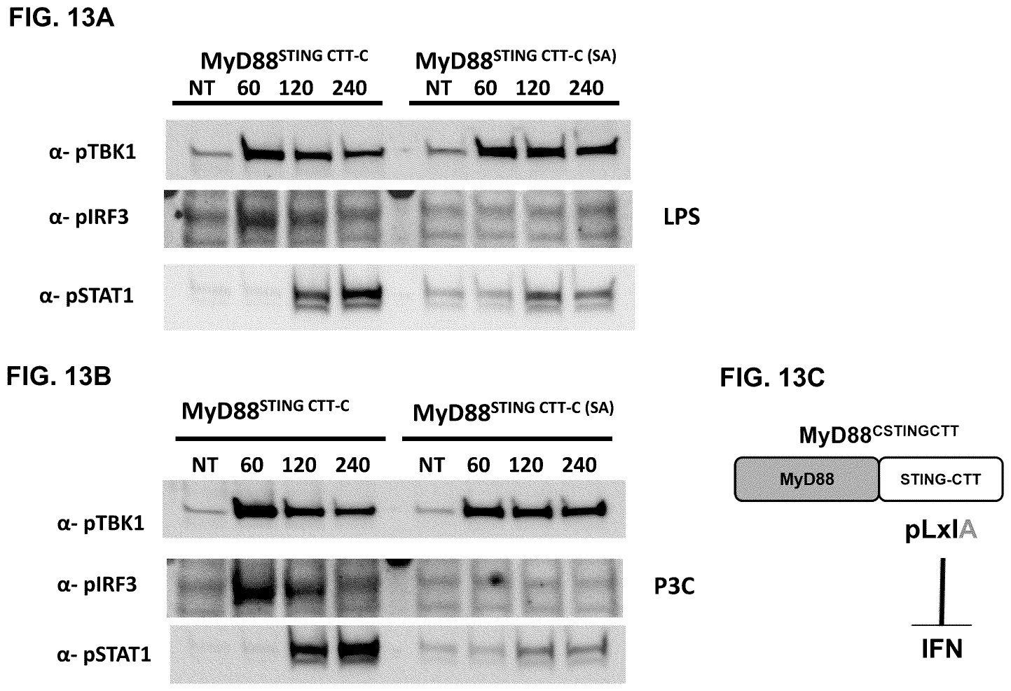

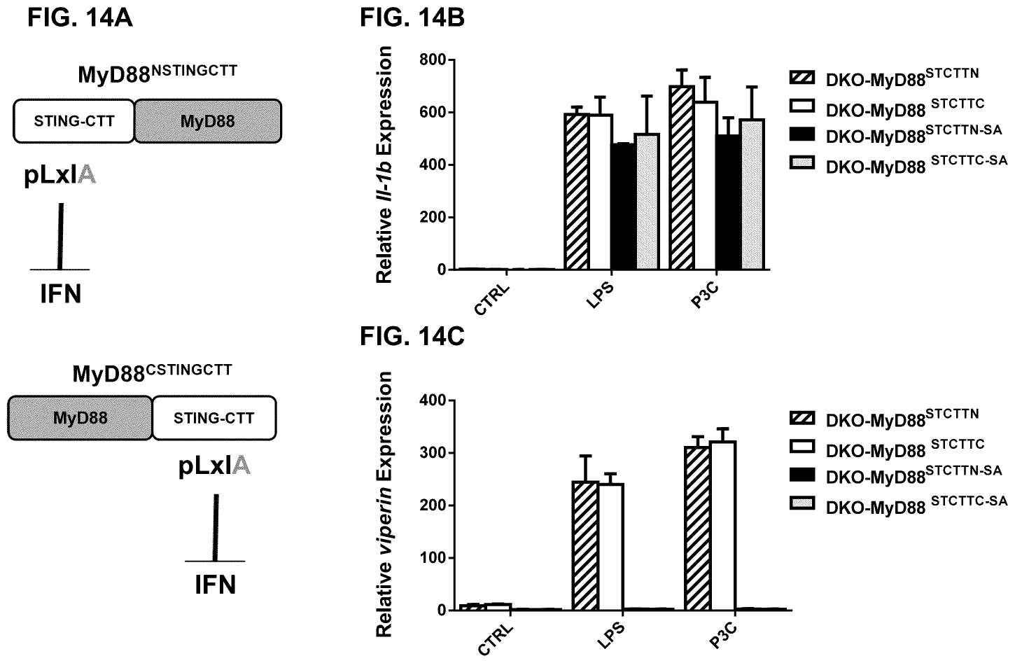

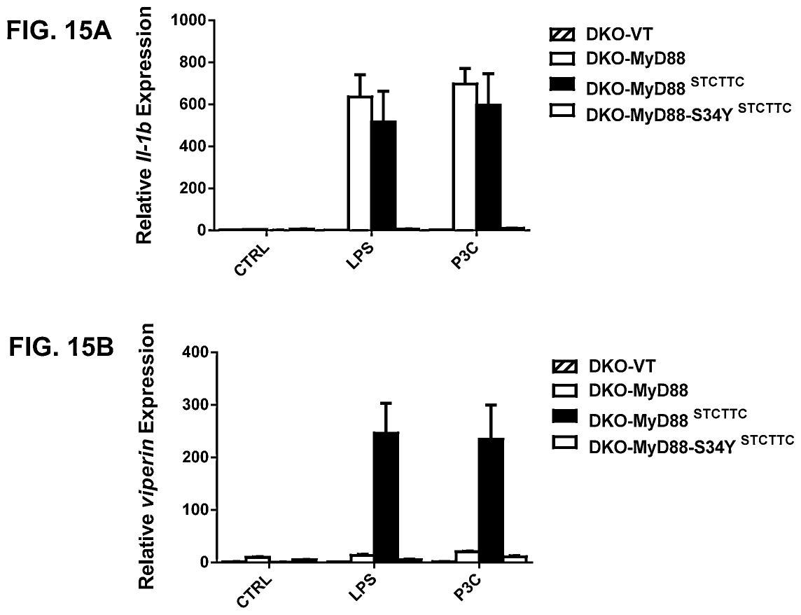

Overexpression of MyD88-pLxIS alleles induced IRF3 phosphorylation (FIG. 5). MyD88-pLxIS alleles restored TBK1 phosphorylation and induced IRF3 phosphorylation in response to LPS and P3C (FIG. 6). MyD88-pLxIS alleles rescued Ill-b and viperin expression in response to LPS and P3C (FIG. 7). Expression levels of MyD88 alleles were comparable to that of endogenous MyD88 in WT iBMDMs (FIG. 8). TBK1 activity was required for IRF3 phosphorylation induced by MyD88-pLxIS alleles (FIG. 9). Phosphorylation of the Ser residue in the pLxIS motif by TBK1 was essential for IRF3 activation (FIG. 10). TBK1 phosphorylated STING-CTT at 5365, which further recruited IRF3 (FIG. 11). L373 was also directly involved in IRF3 recruitment, and neither S365 nor L373 was required for STING-TBK1 interaction (FIGS. 12-14). MyD88 oligomerization/myddosome formation was required for IFN responses (FIG. 15).

### Example 3: The Myddosome can be Rewired to Triger Distinct Forms of Cell Death

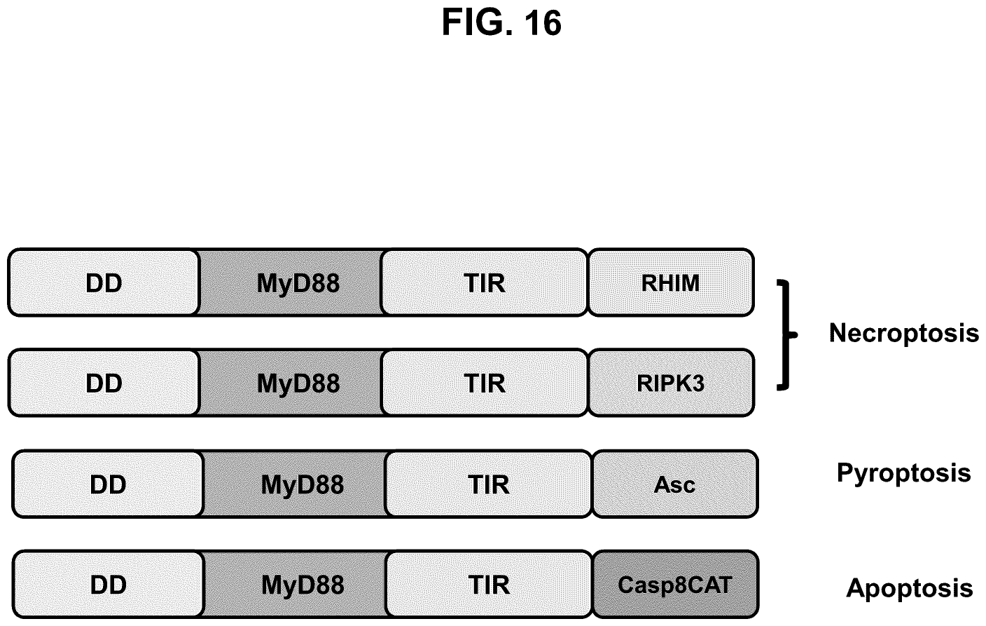

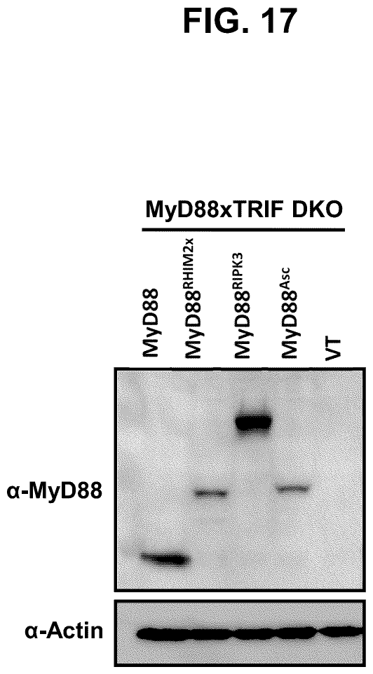

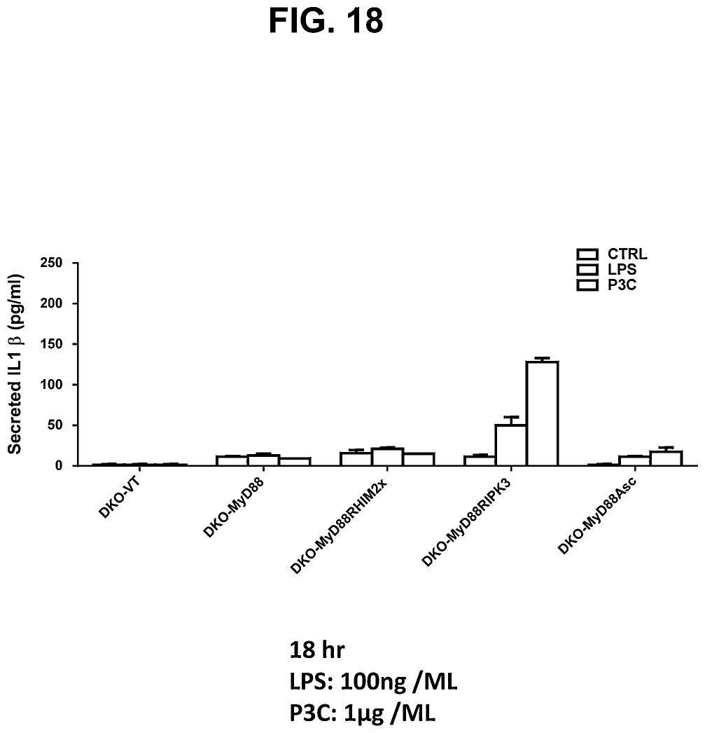

The myddosome can be rewired to trigger distinct forms of cell death, i.e., during necroptosis, pyroptosis or apoptosis (FIG. 16). Expression levels of distinct MyD88-death alleles in MyD88×TRIF DKO iBMDMs was observed (FIG. 17). MyD88-RIPK3 allele in MyD88×TRIF DKO iBMDMs led to IL-10 Release upon LPS and P3C Stimulation (FIG. 18).

### Example 4: Summary

By engineering a synthetic gene where the interferon inducing domain (known as pLxIS) is fused to the coding sequence of the TLR/IL-1 pathway regulator MyD88, a hybrid protein was produced that has the unique ability to induce cytokines and interferons.

By engineering a synthetic gene where a death inducing domain (known as RHIM) from the kinase RIPK3 is fused to the coding sequence of MyD88, a hybrid protein was produced that has the unique ability to link TLR/IL-1 activation to cell death.

By engineering a synthetic gene where the pLxIS is fused to the coding sequence of the inflammasome regulator ASC, a hybrid protein was produced that has the unique ability to link inflammasome assembly to interferon expression.

Any analogous strategies are foreseen, and these examples provide an overview of the breadth and capabilities this approach—to create synthetic immune signaling pathways that combine beneficial activities into one.

### Example 5: Alternative Embodiments

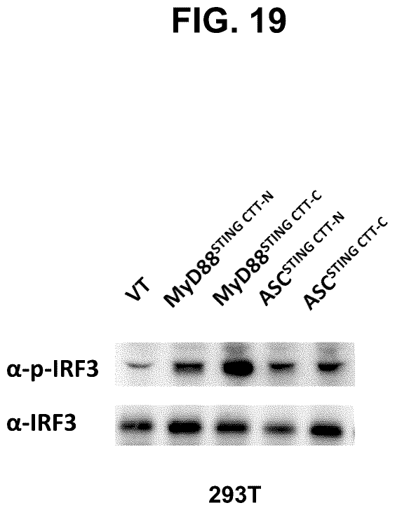

Adaptor proteins of SMOCs were versatile platforms for rewiring signaling circuits. ASC-STING chimeric constructs induced IRF3 Phosphorylation when overexpressed in 293T cells (FIG. 19).

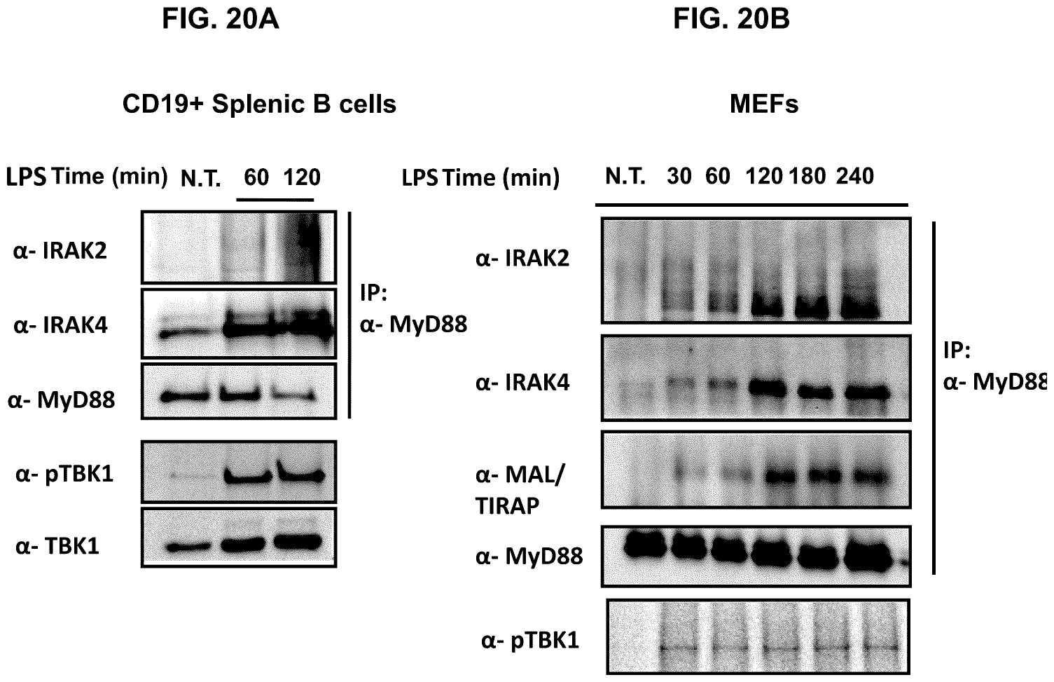

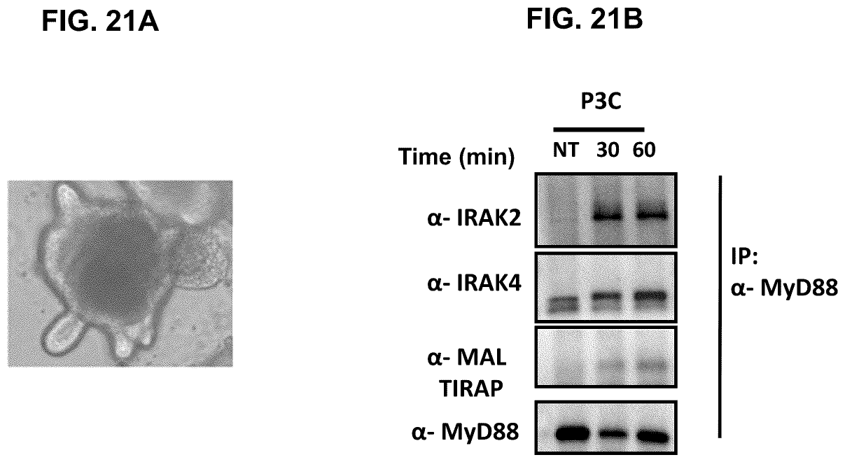

Diverse cell types form myddosome upon TLR activation (FIGS. 20 and 21). Mouse colon crypts were extracted using 5 mM EDTA (tissue washed 10× in cold PBS before extraction in cold EDTA). LGR5+ stem cells were then cultured from the crypts in matrigel in the presence of 50% L-WRN sups (20% FBS Advanced DMEM F12 plus glut and p/s) and 10 μM ROCK inhibitor. 3D stem cell cultures (grown for 3 days from individual LGR5+ cells) were differentiated into organoids over 3 to 5 days in the absence of L-WRN. Organoids (Colonoids) were extracted from matrigel and stimulated in DMEM F12 with ligands.

The data described herein indicated that signal transduction via SMOCs could be reprogrammed. Furthermore, the myddosome is not restricted to professional phagocytes, and is likely to have cell-type specific functions.

In further examples, the synthetic genes described herein can manipulate (alternatively, rewire) immune pathways, induce inflammation, induce cell death pathways, induce interferon expression, induce the inflammasome pathway, and the like. In further examples, the synthetic genes described herein can induce or suppress cell division, differentiation, and cell-cell communication, and migration, phagocytosis, and the like.

### Example 6: TLR Signaling Induced Aerobic Glycolysis, MyD88-Dependent TBK1 Activation Promoted Akt-Mediated Glycolytic Burst Independent of IFN Production, and TBK1 was a Novel Component of My Myddosome and Diversified the Functional Outcomes of this SMOC Beyond NF-kB Activation

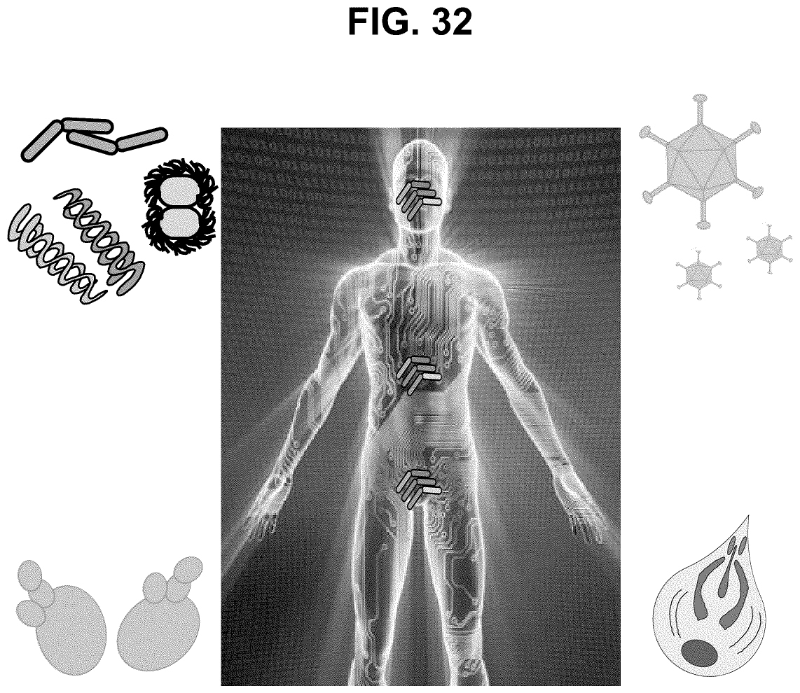

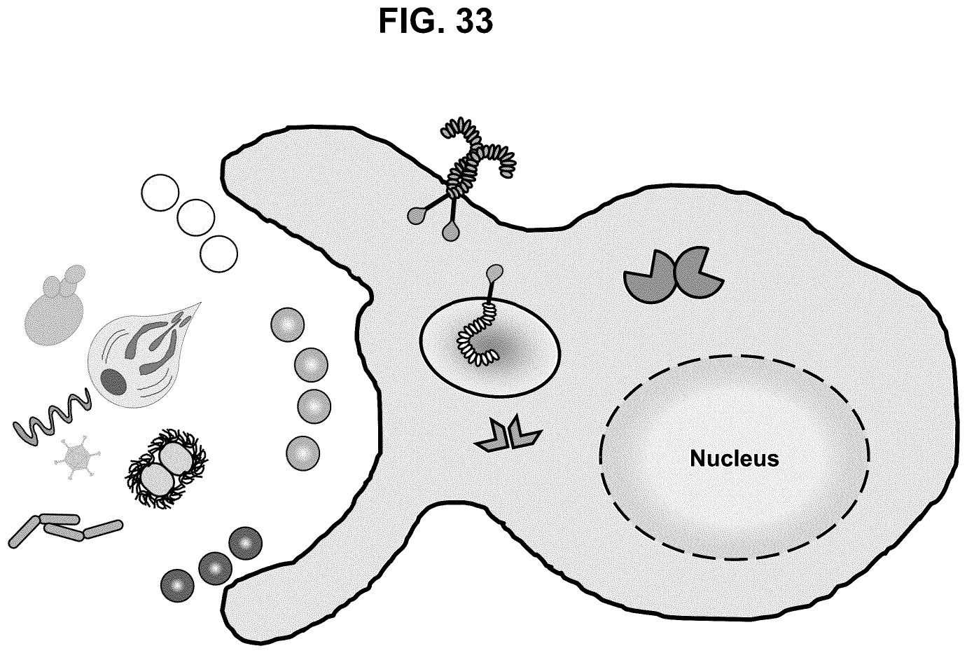

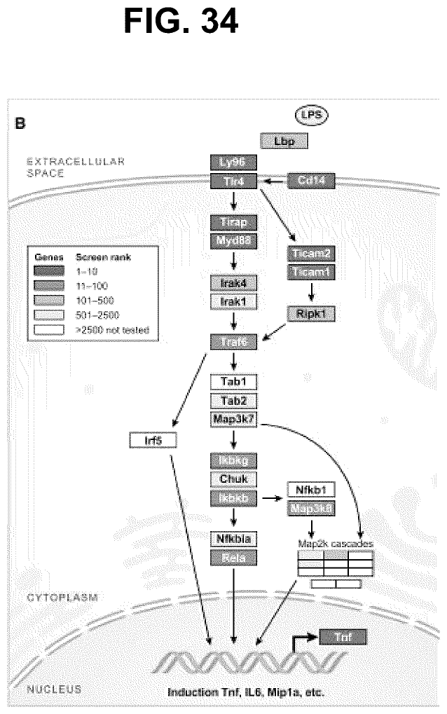

Pattern recognition receptors recognize microbe associated molecular patterns (FIG. 33). Toll-like Receptor (TLR) Family is one of the best genetically defined PRR families; for example, a genome-wide CRISPR screen in primary immune cells to dissect regulatory networks (FIG. 34).

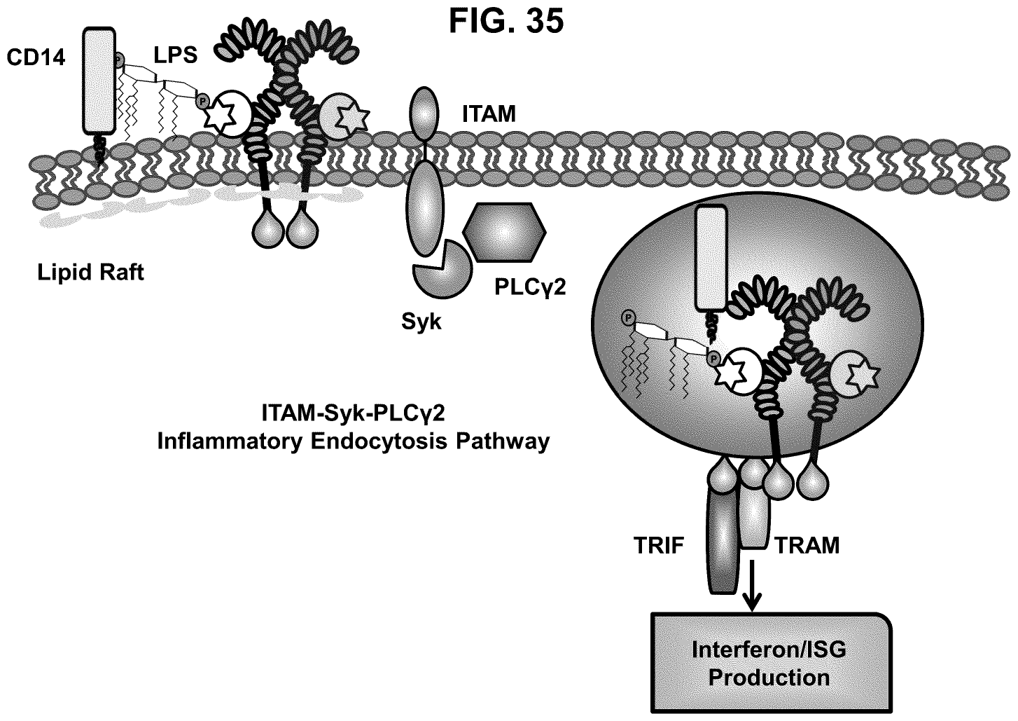

CD14 controls TLR4 endocytosis and MD-2 selects TLR4 as cargo. GPI anchor protein CD14 was identified to activate an endocytosis pathway composed of ITAM adaptors, Syk kinase, and phospholipase Cr2 to bring TLR4 in to the cell. Strikingly, TLR4 signaling was not required for this endocytosis event (FIG. 35).

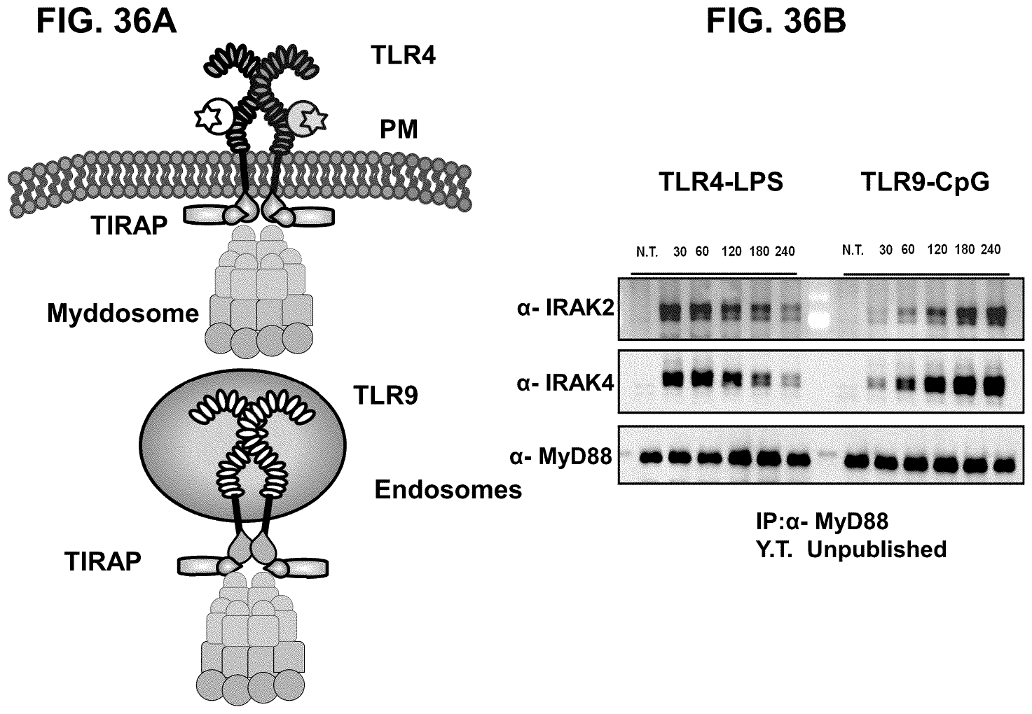

TIRAP was the first cellular regulator of the myddosome (FIG. 36).

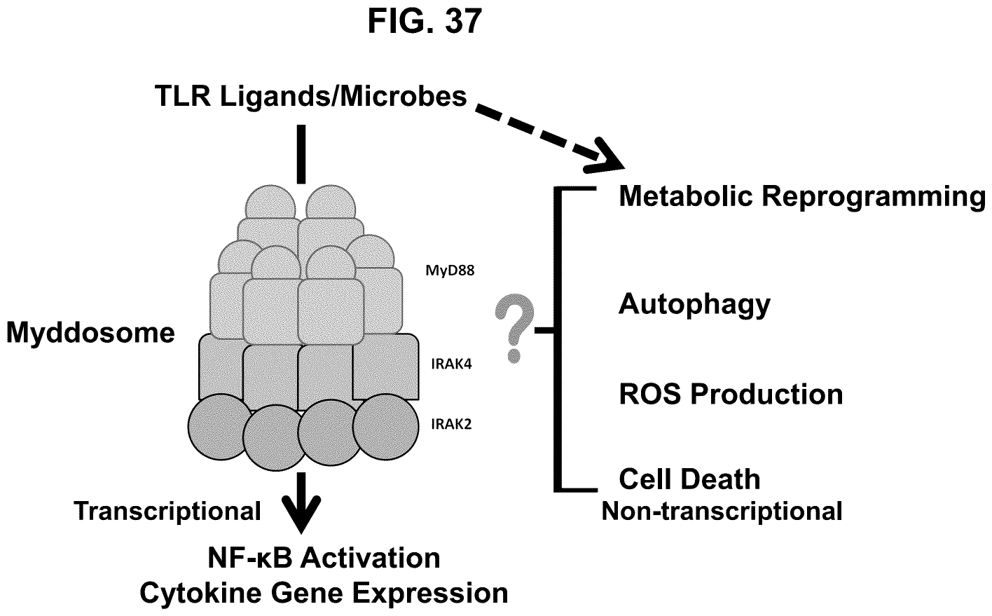

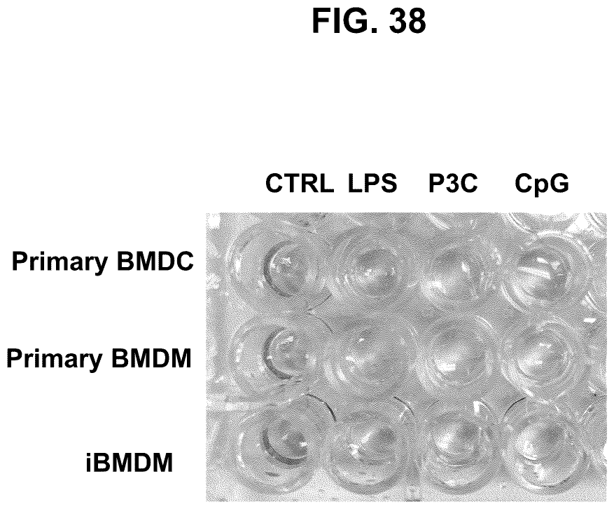

The Myddosome Functions as a Signaling Platform to Coordinate Diverse Cellular Processes Upon TLR Activation; in addition to the well-characterized transcriptional responses, TLR pathway activation has also been implicated in diverse host responses such as metabolic reprogramming, autophagy ROS production cell death etc, which are non-transcriptional responses. Whereas myddosome formation is known to activate NF-kB activation, it is largely unclear whether Myddosome formation induces these other responses. Therefore, the Myddosome is a signaling platform that coordinate diverse cellular processes upon TLR activation was hypothesized (FIG. 37). It was shown that TLR activation induces media acidification in primary and immortalized cells (FIG. 38).

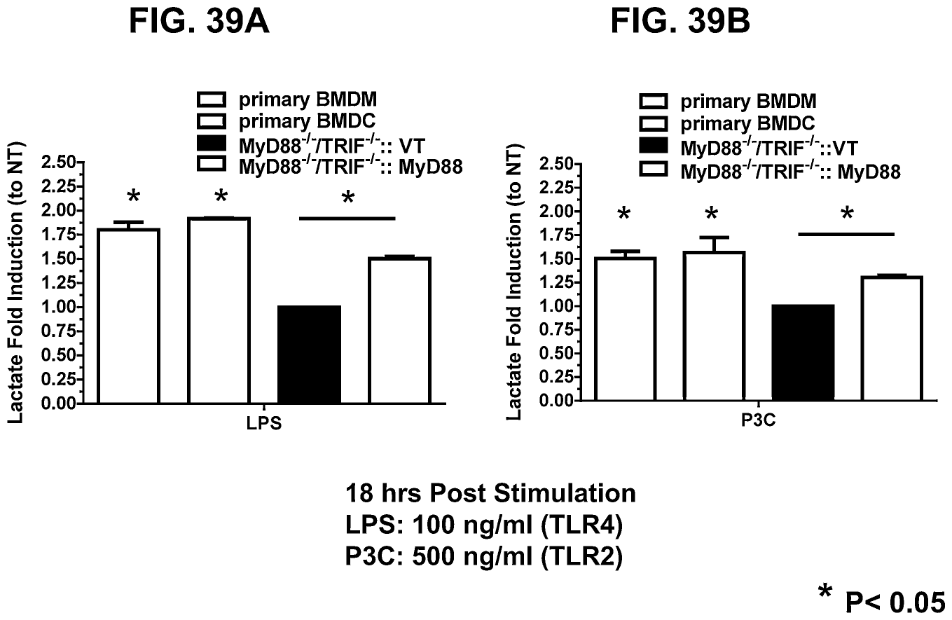

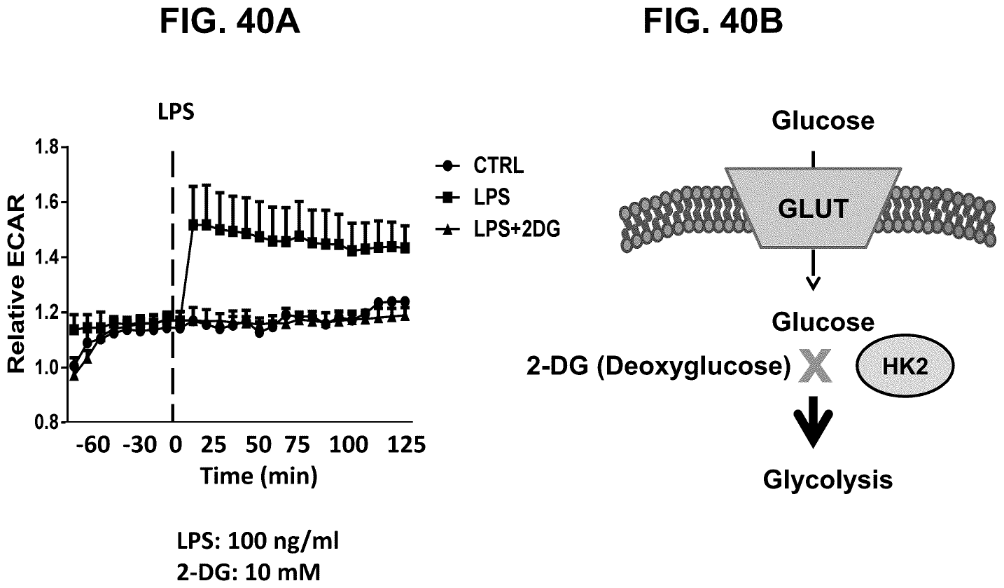

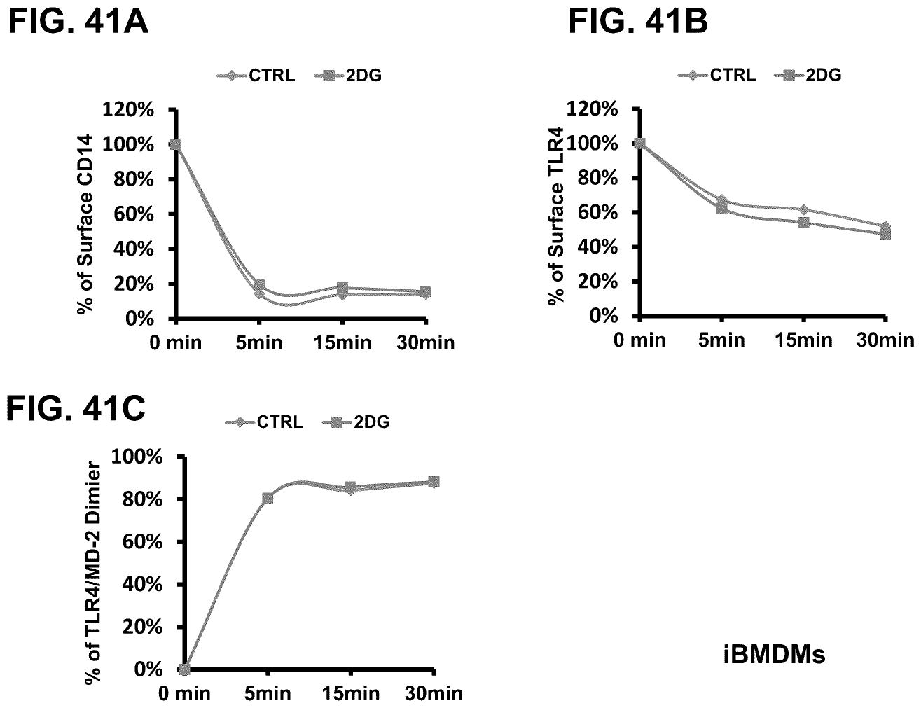

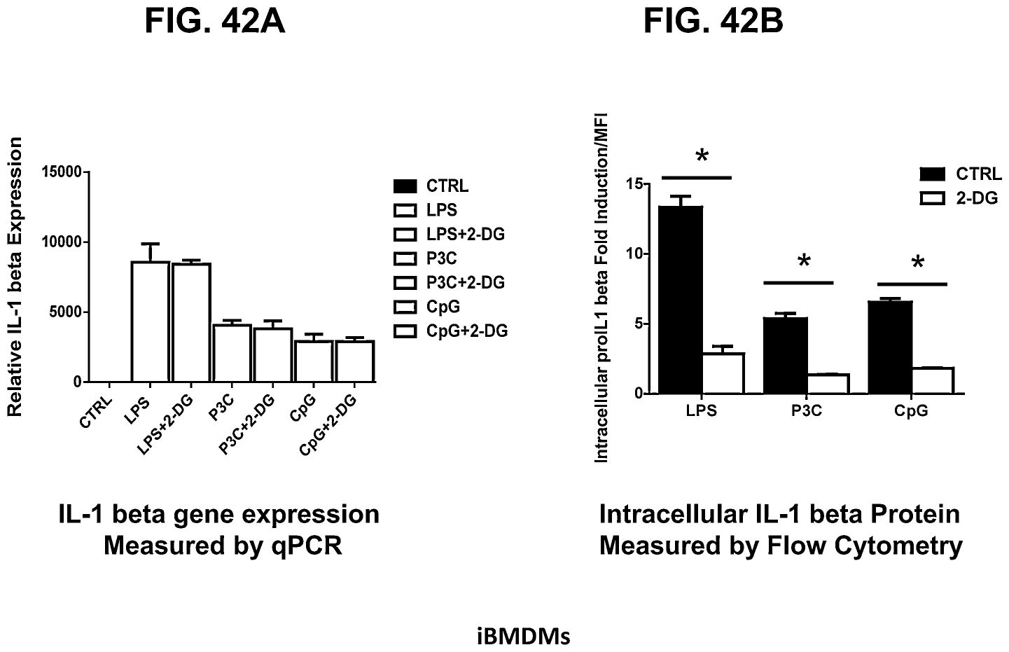

TLR activation promoted glycolysis in primary and immortalized cells (FIGS. 39A-39B), and TLR activation promoted rapid glycolytic burst in iBMDMs (FIG. 40). Also, 2-DG treatment did not affect host responses at the receptor proximal (FIGS. 41A-41C). Inhibition of glycolysis by 2-DG uncoupled cytokine gene transcription from translation (FIGS. 42A-42B).

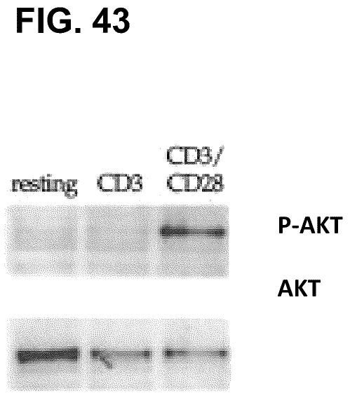

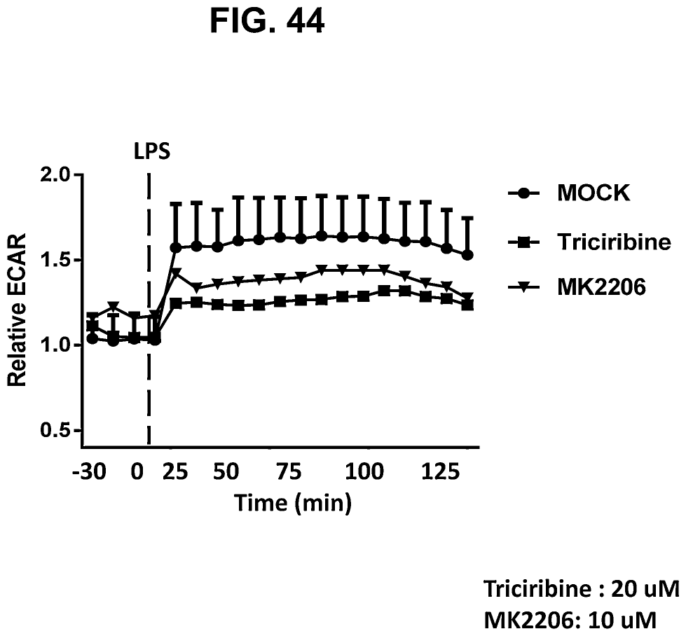

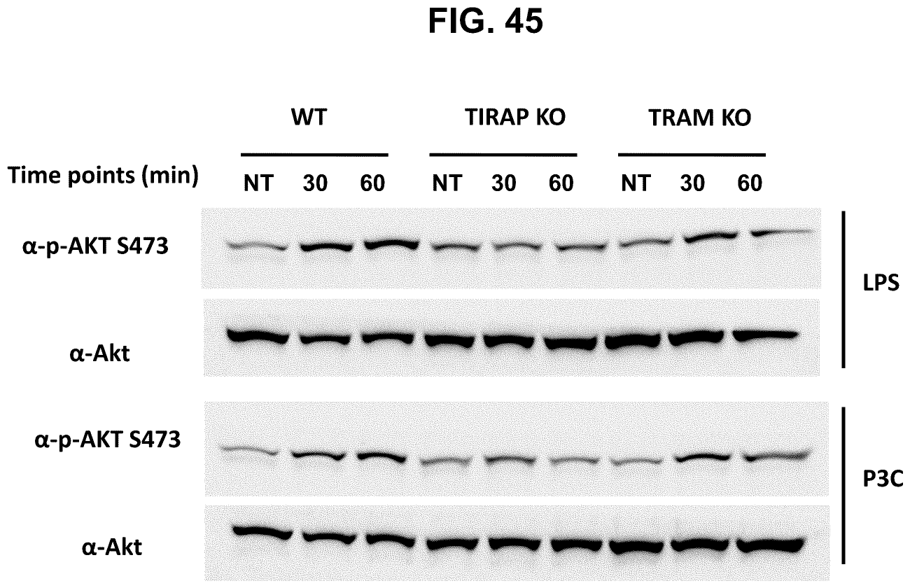

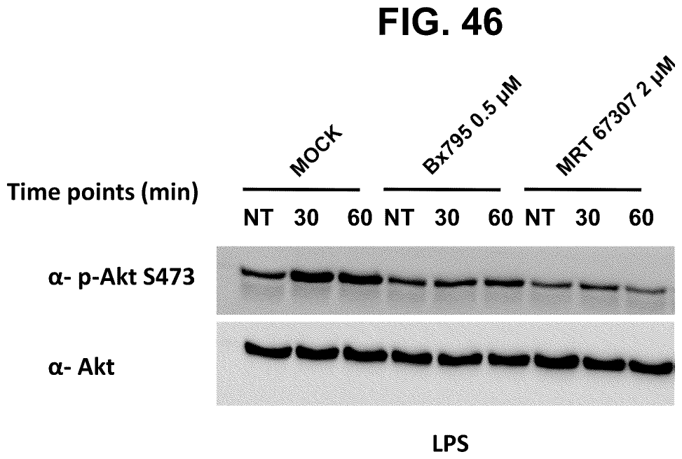

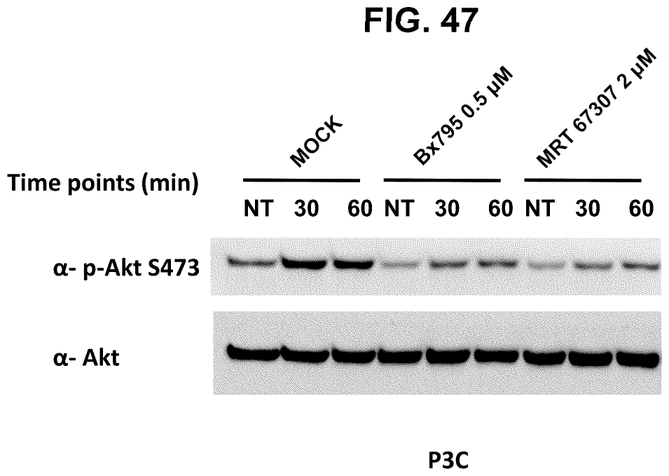

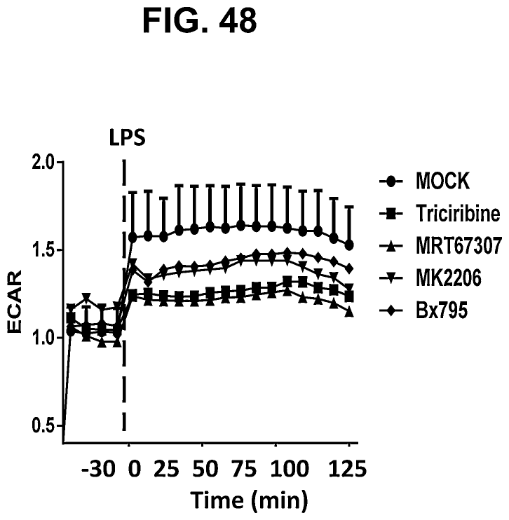

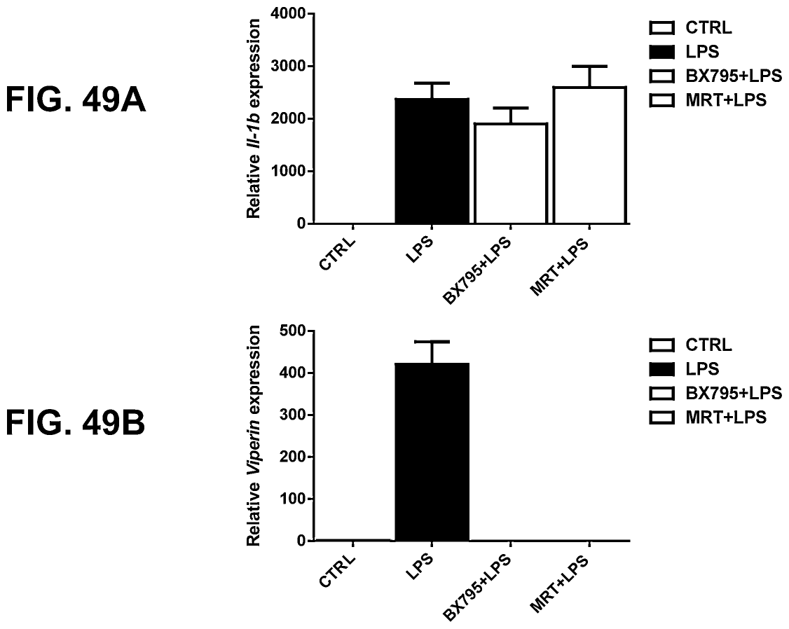

TLR activation induced glycolysis, i.e. that the protein kinase Akt might be critical for early phase TLR-mediated glycolysis (FIG. 43). Inhibition of Akt activation dampened TLR-mediated glycolytic burst (FIG. 44). MyD88 signaling primarily drove Akt phosphorylation (FIG. 45). Chemical inhibitors targeting TBK1 activity reduced Akt phosphorylation in WT iBMDMs (FIG. 46 and FIG. 47). Chemical inhibitors of TBK1/IKKe and AKT dampened TLR dependent glycolysis activation (FIG. 48). TBK1 inhibitors did not affect NF-kB activation at the transcriptional level (FIGS. 49A-49B).

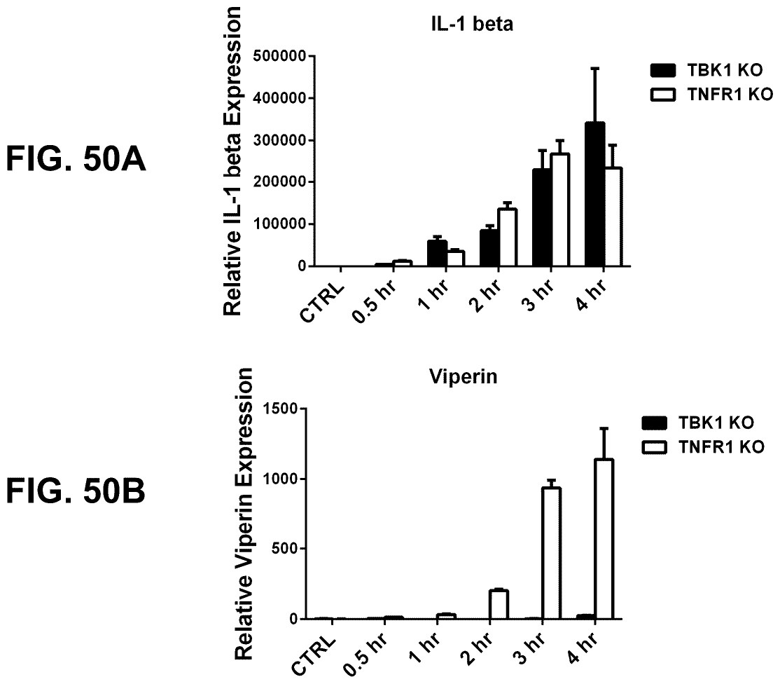

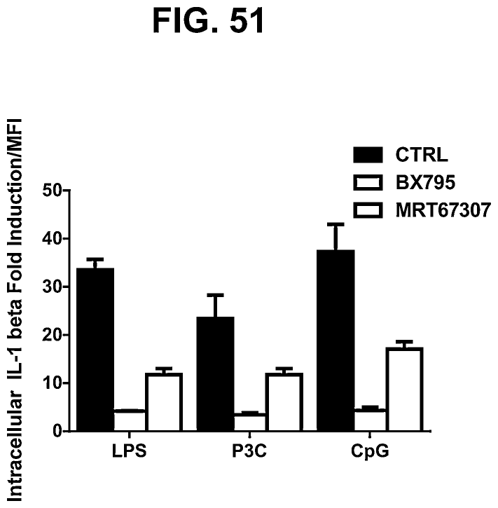

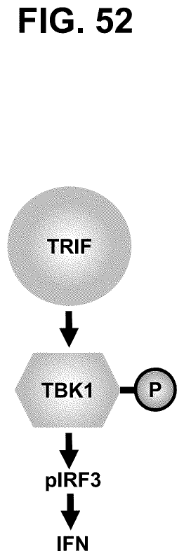

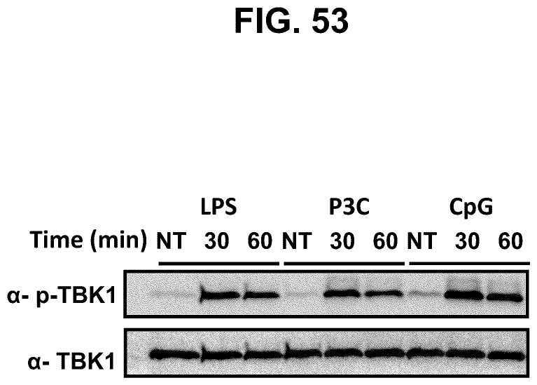

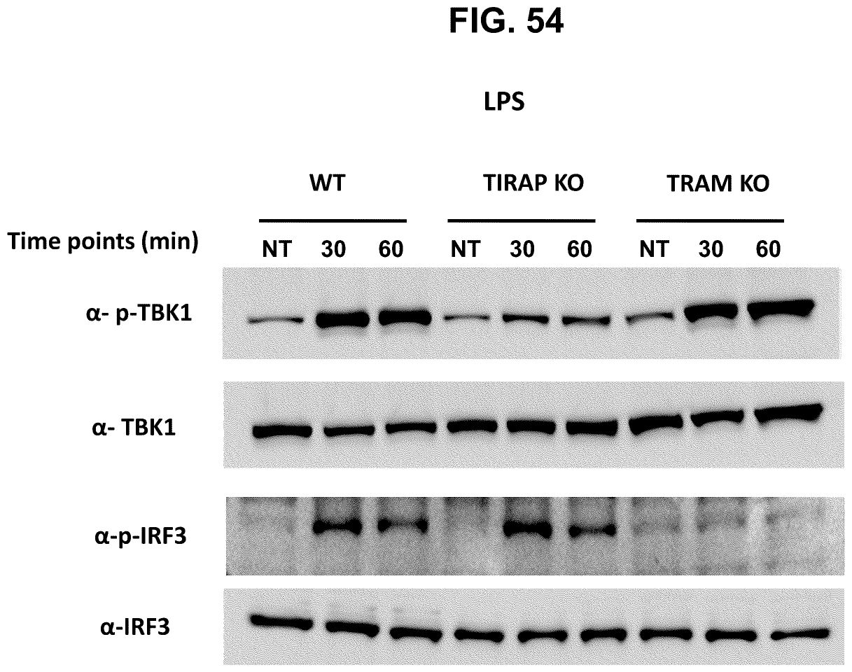

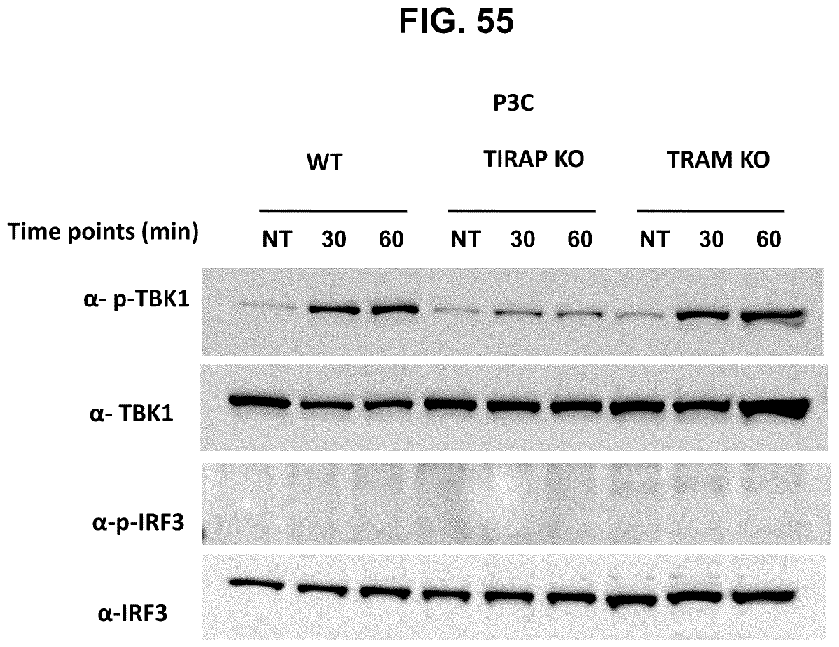

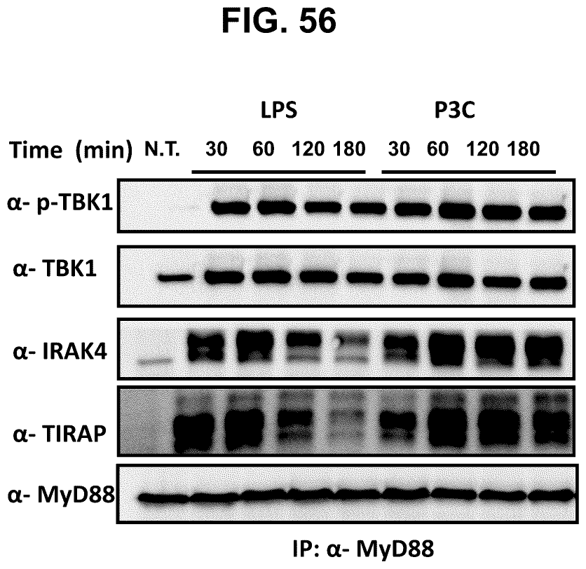

It was shown that TBK1 was dispensable for pro-inflammatory cytokine gene expression (FIGS. 50A-50B). Pro-inflammatory cytokine production was inhibited by chemical inhibitors of TBK1/IKKe (FIG. 51). TLR signaling promoted TBK1 phosphorylation independent of TRIF-IRF3 signaling axis (FIG. 53). MyD88 signaling promoted efficient TBK1 phosphorylation in the TLR4 and TLR2 pathway (FIG. 54 and FIG. 55, respectively). TBK1 was associated with the Myddosome in responses to surface TLR ligands (FIG. 56).

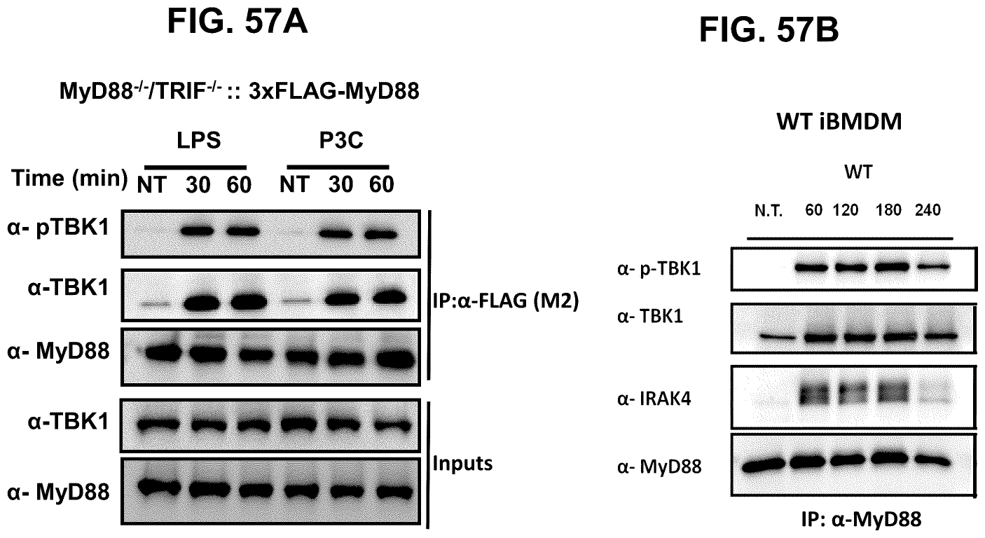

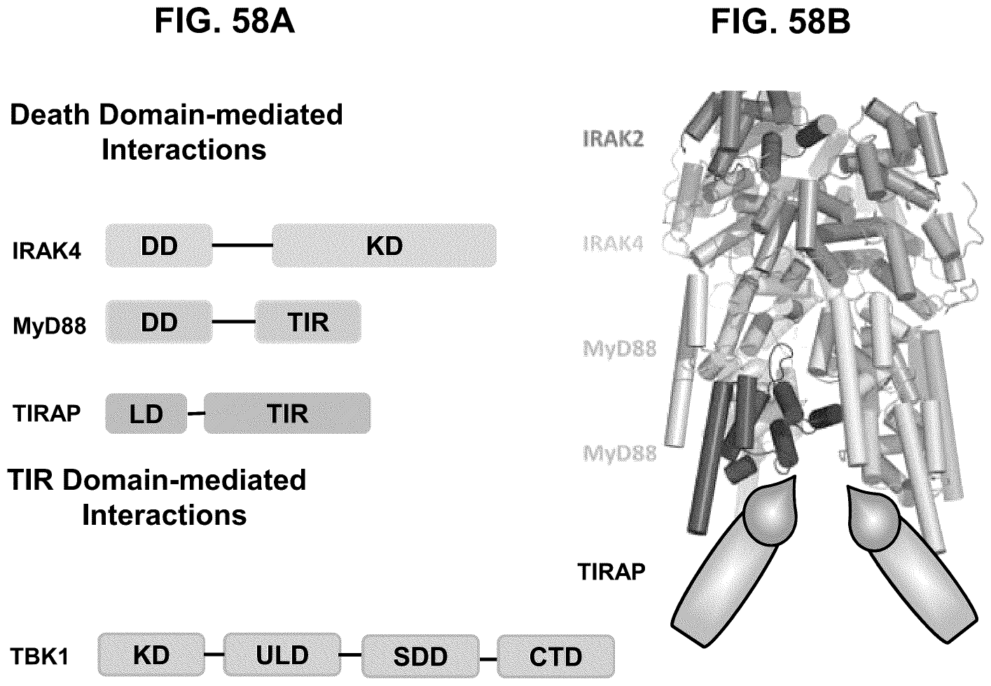

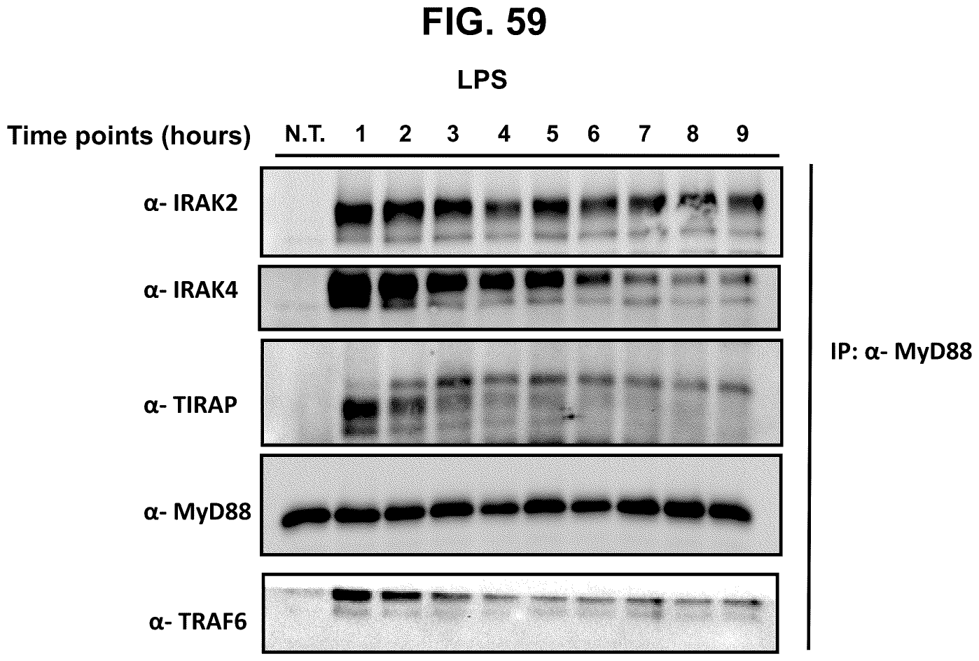

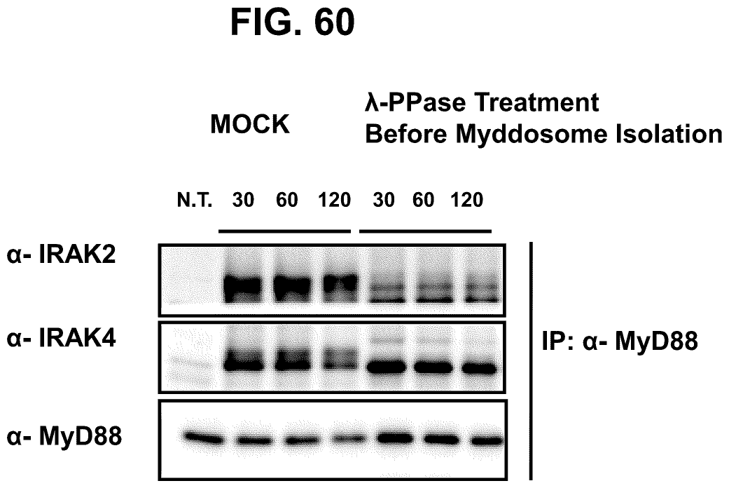

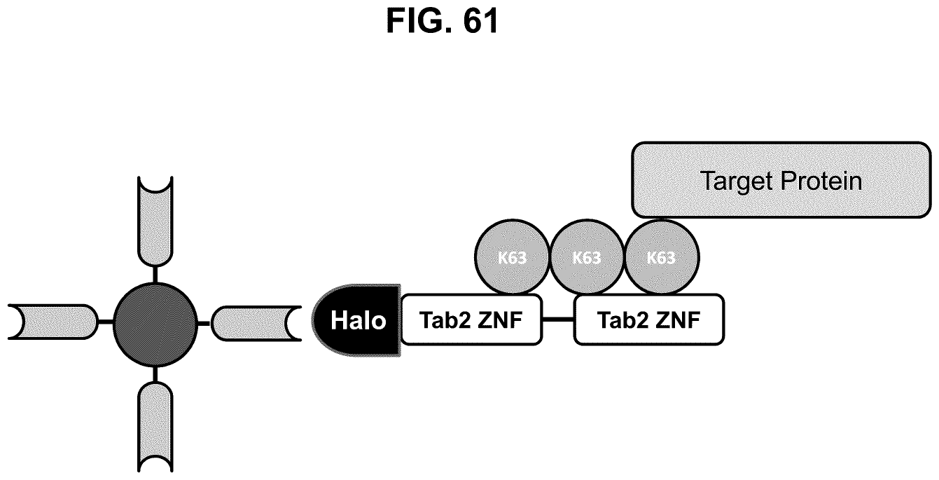

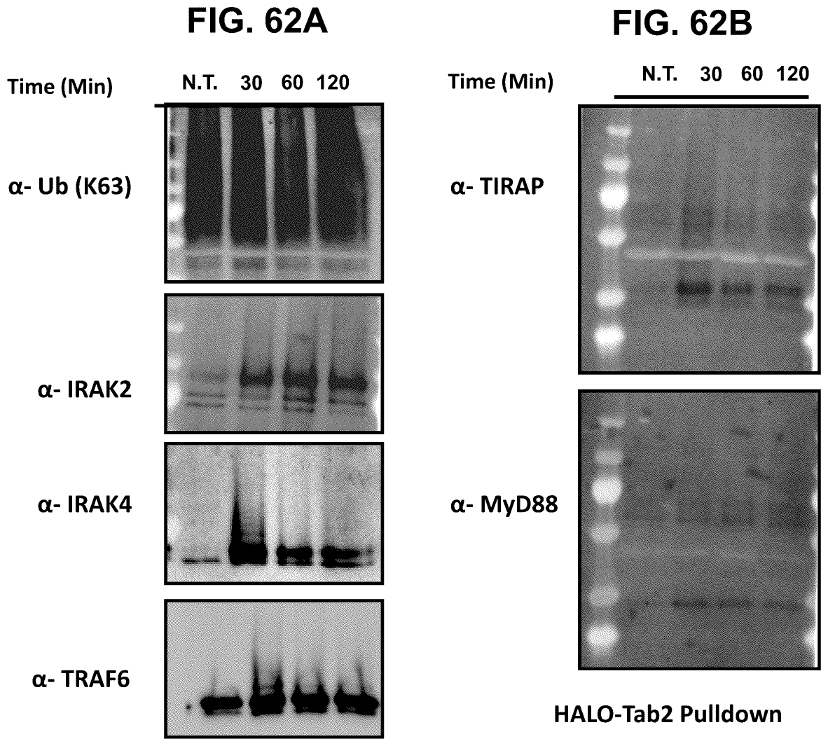

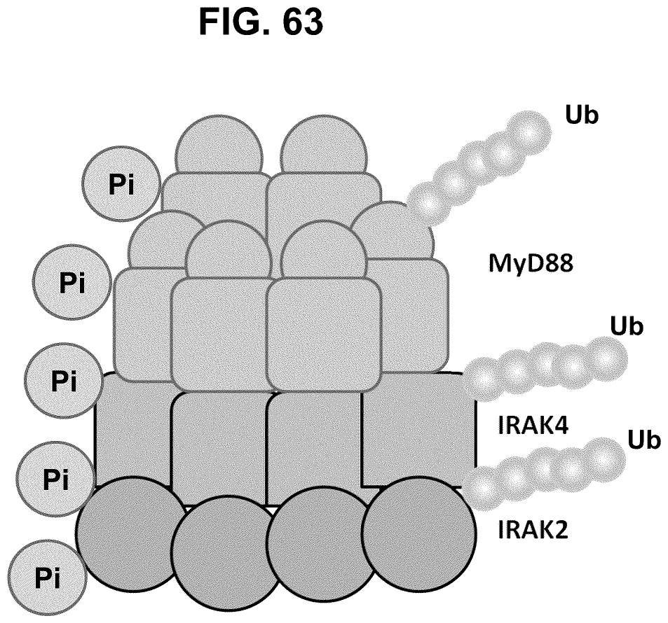

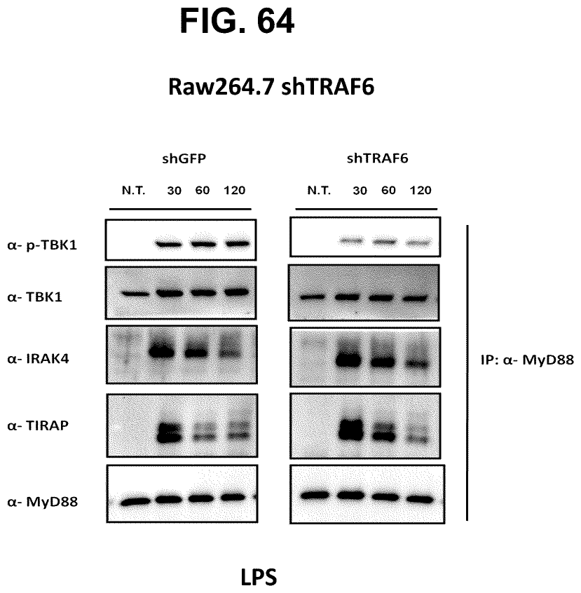

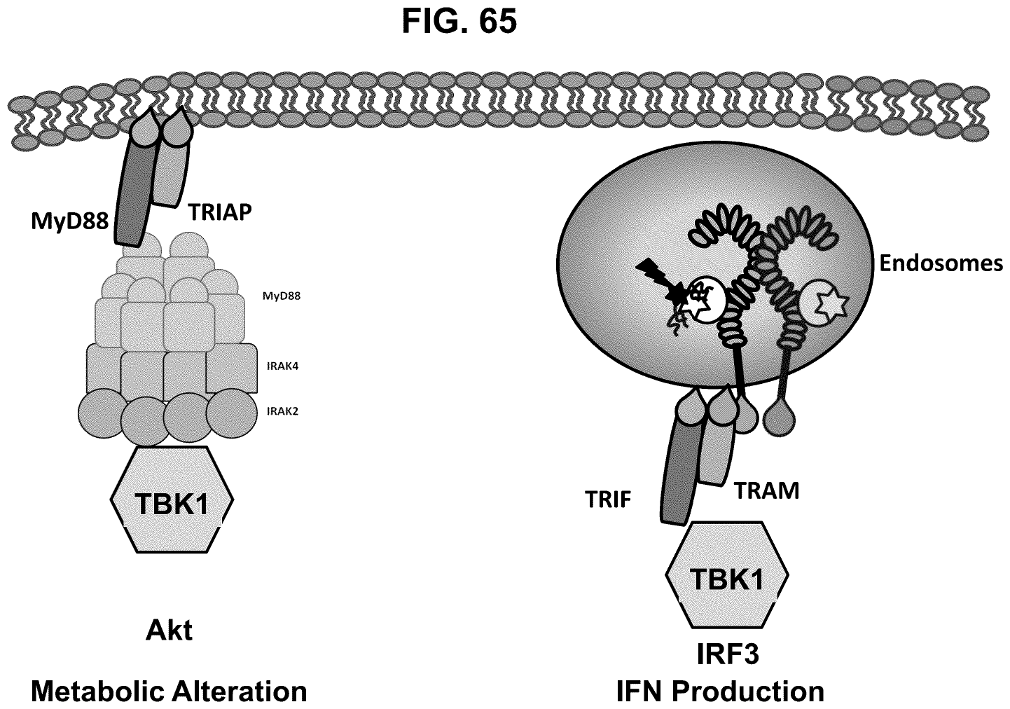

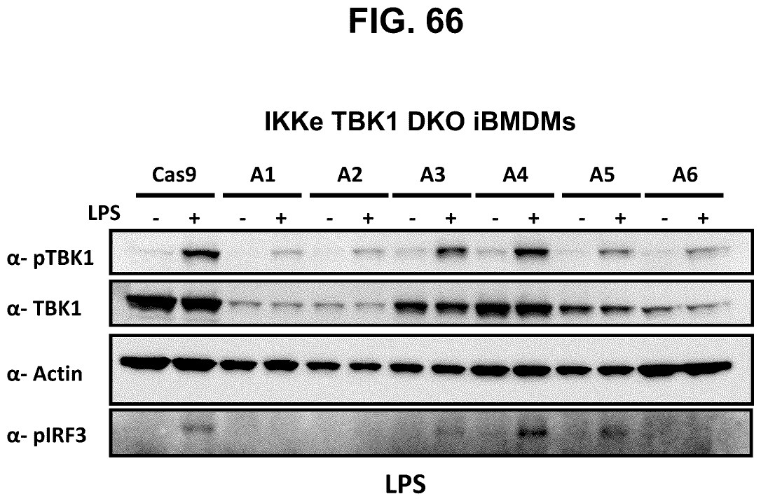

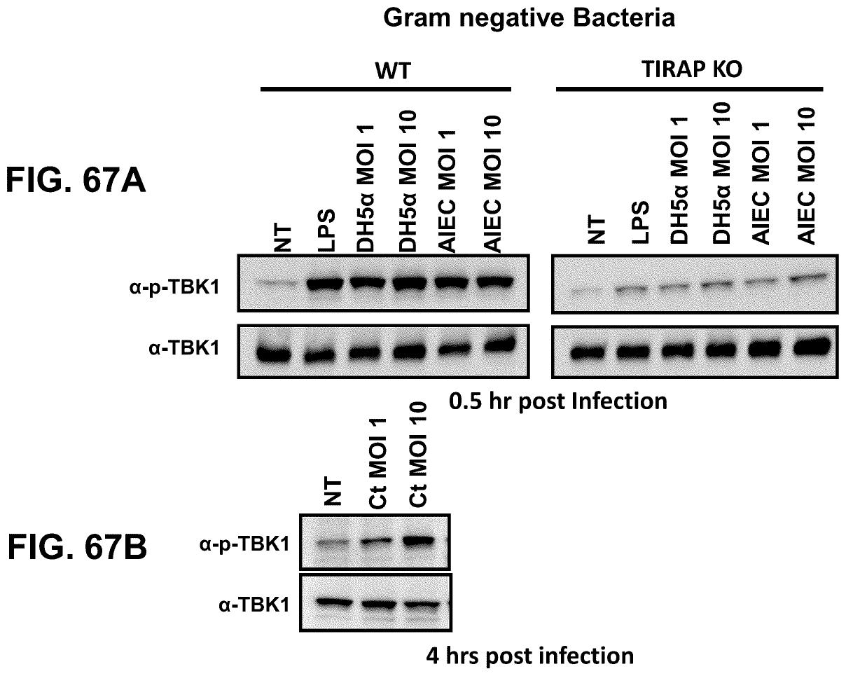

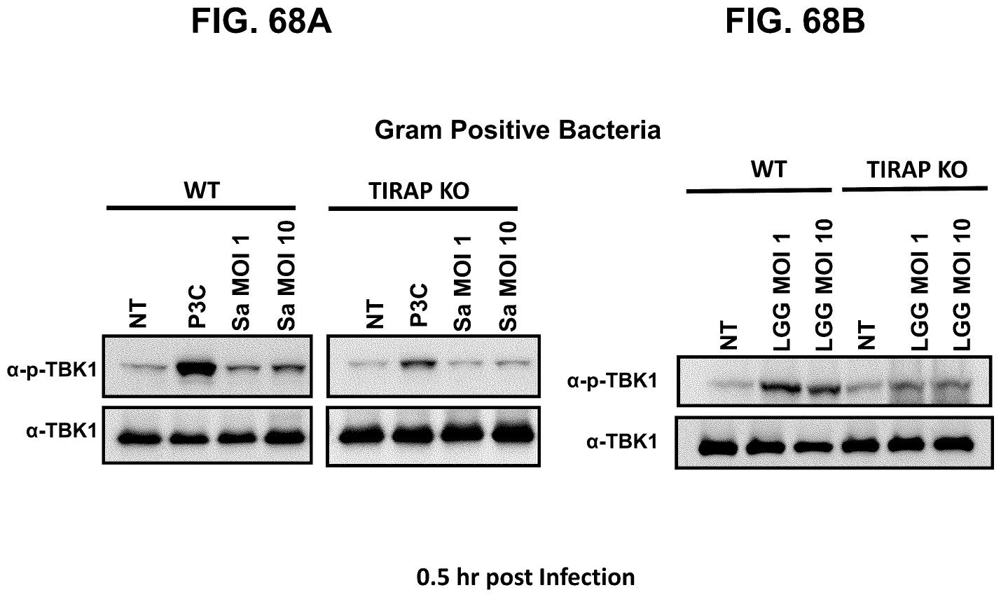

TBK1 was identified as a novel component of the myddosome (FIGS. 57A-57B). The canonical components of the myddosome interacted with each other via homotypic interactions (FIGS. 58A-58B). Myddosome formation in living cells was regulated by distinct post-translational modifications (FIG. 59). Furthermore, components of the myddosome were phosphorylated (FIG. 60). An image depicting whether major components of the myddosome subjected to ubiquitinylation; i.e., halo-Tab2 pulldown: an Affinity purification strategy to isolate K63-ubiquitinylated proteins is shown in FIG. 61. Also, myddosome components were associated with ubiquitin chains (K63) (FIGS. 62A-62B). Living cells post-translational modifications created platforms for protein-protein interactions (FIG. 63). TRAF6 might regulate TBK1 phosphorylation (FIG. 63), and the distinct biological roles of TBK1 in TLR signaling is shown in FIG. 63. The validation of the observations from chemical perturbation of TBK1 function with genetics is shown in FIG. 66. TBK1 was activated upon microbial encounters (FIGS. 67A-67B, and FIG. 68A-68B).

### Example 7 Toll-Like Receptors Enlist a Multifunctional Signaling Organelle to Drive Diverse and Programmable Innate Immune Responses

The ability to detect and respond to environmental stresses represents one of the key features of living organisms. In the context of host-pathogen interactions, the innate immune system provides a faithful illustration to this principle of life, as failure to rapidly sense or respond to pathogens would cast a fatal stress on the host (Pandey et al., 2014).

Microbial sensing, at the cellular level, is achieved by a large number of structurally unrelated proteins that are collectively known as pattern recognition receptors (PRRs) (Janeway, 1989). These receptors detect the presence of conserved structural components or activities uniquely associated with pathogens, which are referred to as pathogen associated molecular patterns (PAMPs) (Pandey et al., 2014). Detection of PAMPs and other microbial activities by PRRs engages numerous cellular processes to eliminate infection and restore homeostasis (Vance et al., 2009).

Based on their primary sequence homology, a majority of the PRRs can be categorized into groups, which include the Toll-like receptors (TLRs), the C-type lectin receptors (CLRs), the Nucleotide-binding domain, leucine rich repeat (LRR)-containing proteins (NLRs), and the AIM2-like receptors (ALRs) (Brubaker et al., 2015).

Genetic analysis over the last two decades has revealed that upon microbial detection, distinct PRRs engage numerous signaling proteins to activate various host defense mechanisms (Kagan and Barton, 2015). Thus, the innate immune system is considered a highly complex entity. Within this complexity of proteins and regulatory factors, unifying themes may exist that govern the operation of immune signaling pathways. However, such themes have only been identified at the level of microbial detection, where the concept of pattern recognition permeates the literature (Medzhitov, 2009). Unifying concepts associated with signal transduction are limiting, as much research has been focused on identifying cellular processes and factors that distinguish one PRR-induced signaling pathway from another (Kagan et al., 2014).

Common themes in innate immune signal transduction may exist (Kagan et al., 2014). For example, PRRs of the TLR, RLR and NLR families seed the formation of large helical oligomeric protein complexes that consist of a receptor, an adaptor and an effector enzyme (Kagan et al., 2014). In the TLR pathway, the oligomeric complex is known as the myddosome, and consists of a TLR, the adaptors TIRAP and MyD88 and enzymes of the IRAK family of serine threonine kinase (Bonham et al., 2014; Lin et al., 2010; Ve et al., 2017). In the NLR pathway, the best-defined oligomeric complex is the inflammasome, which commonly consists of an NLR, the adaptor ASC and enzymes of the caspase family of proteases (most commonly caspase-1) (Cai et al., 2014; Hu et al., 2015; Lu et al., 2014). Finally, the oligomeric complex associated with RLR signal transduction consists of the receptor, the MAVS adaptor and the enzyme Tank Binding Kinase-1 (TBK1) (Jiang et al., 2012; Peisley et al., 2013). While these complexes share the physiological activity of regulating host defense, they do not currently share any components (Kagan et al., 2014). Convergent evolution may have therefore driven multiple unrelated proteins to organize themselves into a common structure that executes host defense mechanisms (Medzhitov, 2009). Why would such a protein complex be commonly utilized by the innate immune system? One possible explanation is that these complexes provide a biochemical scaffold that is modular by design, such that diverse upstream inputs (microbes) can induce their assembly. Once assembled, diverse downstream outputs (defense mechanisms) can be induced. This idea prompted the classification of these structures as supramolecular organizing centers (SMOCs), which represent the principal subcellular sites of signal transduction and are therefore considered the signaling organelles of the innate immune system (Kagan et al., 2014).

However, experimental evidence supporting this speculation has remained sparse. While it is clear that diverse microbes induce assembly of the myddosome, the inflammasome and the RLR-MAVS complex, whether these complexes serve as the site of diverse effector responses is unclear. These gaps of knowledge are due to our incomplete understanding of the composition and regulation SMOCs within cells.

The TLR-induced myddosome is an excellent model to examine the central prediction of the SMOC hypothesis—that these structures represent sites where diverse effector responses emanate. TLRs are type I transmembrane proteins that reside on the plasma membrane and endosomes (Pandey et al., 2014). They detect a wide range of microbial products including bacterial lipopolysaccharides (LPS), lipoproteins, flagellin and nucleic acids (Pandey et al., 2014). Signal transduction in the TLR pathway is regulated by two SMOCs—the aforementioned myddosome and the poorly-defined triffosome (Gay et al., 2014; Lin et al., 2010). The core of the myddosome contains the well-studied adaptor protein MyD88, and the core of the triffosome is thought to contain the adaptor TRIF (Gay et al., 2011; Gay et al., 2014). All TLRs induce MyD88-dependent responses, except for TLR3, leading to activation of the inflammatory transcription factors NF-κB and AP-1 (Gay et al., 2014; Medzhitov and Horng, 2009). The triffosome is thought to be assembled by TLR3 or TLR4 to enhance myddosome-dependent NF-κB and AP-1 activation, and to drive type I IFN expression (Gay et al., 2014). Triffosome-induced IFN expression is linked to its unique ability to prompt TBK1 to activate the IFN-inducing transcription factor IRF3 (Fitzgerald et al., 2003; Hemmi et al., 2004; Yamamoto et al., 2003). Notably, MyD88 deficient cells display defects in TBK1 activation, but the mechanisms and consequences of this unexpected activity are unclear, as MyD88 does not activate IRF3 or induce type I IFN (Clark et al., 2011).

In addition to directing pro-inflammatory transcriptional programs, TLR activation triggers prominent alterations in the cellular metabolic state (O'Neill et al., 2016). Such metabolic reprogramming is exemplified by the TLR-dependent rapid activation of glycolysis (Everts et al., 2014). These metabolic shift is essential for the cells to accommodate to the increased needs for cytokine mRNA translation and secretion (Everts et al., 2014). While glycolysis induction is increasingly recognized for its importance in inflammation, the means by which TLRs promote this effector response is unknown (Everts et al., 2014). In particular, the relative roles of the myddosome and triffosome in directing glycolysis is unclear. Also unclear is whether signals within these SMOCs drive glycolysis directly, or indirectly through the upregulation of genes encoding glycolysis-regulatory factors (Tannahill et al., 2013).

Herein, direct evidence is provided supporting the proposal that SMOCs are organizing centers, in that the myddosome is the source of diverse effector responses induced by TLR activation. We identify a novel component of the myddosome, the kinase TBK1, and find that this kinase is not necessary for early NF-1B or AP-1 activation. Rather, myddosome-associated TBK1 is necessary to induce aerobic glycolysis. Mechanistically, the E3 ligase TRAF6 facilitates the recruitment and activation of TBK1 within the myddosome, which then activates AKT-dependent glycolytic responses. Using synthetic biology approaches, we demonstrate the ability to reprogram the myddosome, in that we have engineered this SMOC to induce type I IFN responses or RIP3-dependent necroptosis in response to TLR ligands. These findings demonstrate the modularity of the effector functions possible within the signaling organelles of our innate immune system.

### Materials and Methods

Cell lines, Transfection, and Retroviral Transduction: Immortalized bone marrow derived macrophages (iBMDMs) were cultured in DMEM containing 10% FBS, Penicillin and Streptomycin (Pen+Strep), and supplements of L-glutamine and sodium pyruvate. PBS (pH 7.4) containing EDTA (2.5 mM) was to detach cells for passage or plate for assays. HEK293T cells were cultured in complete DMEM. Cells were washed in PBS pH 7.4 then detached culture flasks with 0.25% Trypsin. For transient overexpression in HEK293T cells, HA-tagged TBK1 was cloned into the pcDNA vector. MyD88, TIRAP, IRAK2, IRAK4, TRAF6 were cloned into the pEGFPc1 vector, TRAF6 was also cloned into the pCMV-FLAG vector. For retroviral transduction, all TRAF6, MyD88 alleles used in this study were cloned into the pMSCV-IRES-GFP vector.

To generate cell lines stably expressing transgenes, retrovirus particles were produced by transfecting 293T cells with plasmids pCL-Eco, pCMV-VSV-G, and pMSCV-IRES-GFP containing the gene of interest. For lentiviral-mediated shRNA expression, lentiviral particles were produced by transfecting 293T cells with plasmids psPAX2, pCMV-VSV-G, and lentiviral vector expressing TBK1-targeting shRNAs or a control non-targeting scramble shRNA.

Plasmids were transfected into HEK293T cells in 10 cm dishes at a confluency of 50%-70% with lipofectamine 3000 and media was changed 24 hr post transfection and viral supernatants were collected 24 hr post media change. Viral supernatants were spun at 400×g to remove cellular debris, then passed through a 0.45 mm PVDF filter via syringe. Polybrene was added to the filtered supernatants (5 μg/ml), and the supernatants were then used to transduce iBMDMs via spin-fection at 1250×g for 60 min at room temperature. The cell lines were sorted based GFP expression to ensure comparable levels of transgene expression. For shRNA-mediated gene knock down, cell lines stably expressing shRNA constructs were selected by puromycin (20 μg/ml).

Gene expression analysis and ELISA. RNA was isolated from cell cultures using Qiashedder (Qiagen) and GeneJET RNA Purification Kit (Life Technologies). Purified RNA was analyzed for gene expression on a CFX384 real time cycler (Bio-rad) using TaqMan RNA-to-CT 1-Step Kit (Applied Biosystems) with probes specific for Rsad2, II-1b, Il-6 and Gapdh.

ELISA were performed to measure secreted TNFα and IFNβ. Cell culture supernatants were cleared of cell debris by spinning 96 well plates at 400×g for 5 min. Supernatants were transferred to new 96 well plates. Concentrations of TNFα and IFNβ were measured following the manufacturer's protocols.

PI staining and LDH release quantification. For experiments measuring end-point PI staining, PI (5 μM) was included in the culture media to monitor transient pore formation at the last 30 min for each incubation period. A Tecan plate reader was used to measure PI staining (excitation 535 nm, emission 617 nm). Supernatants were assayed for LDH release freshly after stimulation time courses using the Pierce LDH cytotoxicity colorimetric assay kit per the manufacturer's instruction. The same Tecan plate reader was used to measure LDH release (absorbance 490 nm and 680 nm). Cells treated with detergent-containing lysis buffer were used as positive control for PI staining, the resulted supernatants from treated cells were used as positive control for LDH release quantification.

Immortalization Protocol for Bone Marrow Derived Macrophages. Primary BMDMs for immortalization were cultured in complete RPMI with 15% FBS, 30% L929 conditioned supernatant and antibiotics. Conditioned supernatant collected from the CREJ2 cell line carrying the J2 retrovirus was used to immortalize primary BMDMs. In brief, differentiated primary BMDMs (day 7) were further incubated with 50% J2 conditioned supernatant and 50% L929 conditioned supernatant for 7 days, with one new batch of mixed J2 supernatant and L929 supernatant added at day 3. Transduced BMDMs were then cultured in complete DMEM plus 30% L929 supernatant until 90% confluent. Cells were then passed into new medium containing 25% L929 supernatant. Following this trend, the L929 supernatant concentration in complete DMEM was decreased by 5% during each passage. The immortalization process was completed when the BMDMs grew normally in complete DMEM in the absence of L929 supernatant.

Western blotting and immunoprecipitation. For western analysis of signaling pathway activation, primary BMDMs (1×10⁶) or iBMDMs (0.5×10⁶) were seeded in 12 well plates and stimulated with ligands for indicated periods, and subsequently lysed in 300 μl 1×SDS containing 8 M UREA. Lysates were incubated at 65° C. for 15 min. Before SDS-PAGE separation, lysates were passed through a BD 1 ml sub-Q syringe attached to a 26G needle to reduce viscosity. 15 μl of individual samples (15-20 μg protein from whole cell extract) were separated by SDS-PAGE followed by western analysis.

For myddosome isolation, iBMDMs (5×10⁶) were stimulated with ligands for indicated times, and subsequently lysed in 400 μL of lysis buffer containing 50 mM Tris-HCl (pH 7.5), 150 mM NaCl, 5% Glycerol, 1 mM Sodium dioxycholate and 1% NP40. Protease inhibitors and phosphatase inhibitors were added prior to cell lysis. Lysates were spun at top speed for 15 minutes at a table-top centrifuge in the cold room (at 4° C.). The cleared supernatants were collected and 80 μL of the supernatants was saved as sample inputs. 0.5 μg of the anti-MyD88 antibody with 15 15 μl (bed volume) of protein G sepharose (for endogenous MyD88) or 15 μl (bed volume) of anti-FLAG (M2) agarose (for 3×FLAG-MyD88 alleles) was added to the remaining supernatants and the incubation were allowed to proceed for 4-5 hr at 4° C. on a nutator. The beads containing the protein complexes were then washed for 3 times with lysis buffer, and 60 μL of SDS loading buffer was added. The protein complexes were further eluted by heating at 65° C. for 15 minutes. A portion of eluted protein complexes (20 μl) were separated by SDS-PAGE and analyzed by western blot.

For isolation of protein complexes associated with activated TLRs, cells were lysed using a modified lysis buffer containing 50 mM HEPES, 150 mM NaCl, 0.5% DDM (n-Dodecyl-β-D-Maltoside), 0.05% CHS (cholesteryl hemisuccinate). This buffer is suitable for the isolation of membrane protein complexes. 0.5 μg of biotinylated anti-TLR4 (Sal5-21) antibody was used (per sample) to capture the TLR4 signaling complex with streptavidin agarose. 15 μl (bed volume) of anti-HA agarose was used (per sample) to capture the TLR9-HA signaling complex. Immunoprecipitates were washed 3 times with the modified lysis buffer and analyzed by western blot.

Generation of synthetic MyD88 molecules. To generate MyD88-NpLxIS and MyD88 CpLxIS alleles, the cDNA sequence encoding the STING pLxIS motif (340-378 a.a.) was fused in tandem then attached to the cDNA sequence encoding the MyD88 protein either at the 5-prime end (for MyD88-NpLxIS) or at the 3-prime end (for MyD88-CpLxIS). The fusion cDNAs were then synthesized as gBLOCKs via IDT. The mutant alleles were also synthesized as gBLOCKs. To generate the MyD88-RIP3 allele, the cDNA sequence encoding the full-length RIP3 was attached the MyD88-encoding cDNA sequence at the 3-prime end. The fusion cDNA was then synthesized as a gBLOCK via IDT. All synthetic DNA sequences were optimized to avoid internal repeats and for optimal expression in murine cells via the IDT online program.

Generation of the TLR4 (cyto) antibody. The DNA sequence encoding a segment of the TLR4 cytosolic region (660-835 a.a.) was cloned into pQE30. For protein production, 30 mL of the overnight culture of the E. coli strain harboring the appropriate plasmid was transferred to 750 mL LB medium (100 μg/ml ampicillin) and was grown until the OD₆₀₀ value reached 0.6-0.8. After isopropyl β-D-1-thiogalactopyranoside (IPTG) was added to a final concentration of 0.4 mM, the cultures were incubated further in a shaker at 18° C. for 16-18 h. Bacterial cells were harvested by spinning at 6,000×g and were lysed by sonication in the presence of protease inhibitors. The soluble fractions were collected by centrifugation at 12,000×g twice at 4° C. His-tagged proteins were purified with Ni-NTA beads (Qiagen) and eluted with PBS plus 300 mM imidazole.

Polyclonal antibodies against TLR4 (cyto) were generated with recombinant His₆-tagged TLR4 (cyto) as antigen by Pocono Rabbit Farm and Laboratory following standard protocols. Antibodies were then affinity purified using Affigel matrix coated with the antigen.

Microscopy imaging of cell morphology and live cell imaging. To determine cell morphology after TLR stimulation, Myd8^{-/-}/Trif^{-/-} iBMDMs expressing MyD88 and MyD88-RIP3 were seeded in 12-well plates (0.5×10⁶ per well) and were subjected to indicated treatments (Staurosporine 1 μM; TLR ligands 1 μg/ml) to induce cell death. For static image capture, a Nikon Eclipse TS100 microscope was used with 40× magnification. Images were processed with the “NIS-Elements F” software. Representative images were chosen from at least three randomly chosen fields from one representative experiment of three independent experiments.

For live cell imaging, stable iBMDM lines (2×10⁶ per well) seeded into a 35 mm glass bottom dish (Ibidi) were incubated with TLR ligands in PI (5 μM)-containing media. Images were acquired with the Zeiss Axiovert 200M inverted confocal microscope for 1 hr with images taken in every 3 min.

For confocal imaging of CM-induced myddosome formation. Cells were fixed at room temperature for 30 min, permeabilized with 0.2% Triton X-100 for 5 min at room temperature and permeabilized with 2% goat serum in PBS supplemented with 50 mM ammonium chloride. Primary and secondary antibody staining were performed following product instructions. Working concentrations of primary antibodies were used as the following: pTBK1 (1:100), pp 38 (1:100), FLAG (M2) (1:100). Samples were imaged by the Zeiss 880 laser scanning confocal microscope.

Seahorse Metabolic Analysis. Extracellular acidification rate (ECAR) and oxygen consumption rate (OCR) were measured with a Seahorse XFe96 Extracellular Flux Analyzer instrument primary BMDMs and iBMDMs

For real-time experiment, primary BMDMs (5×10⁴ per well) and iBMDMs (7.5×10⁴ per well) were seed in a Seahorse 96 well plate in complete DMEM medium. Cells were allowed to attach to the assay plate for 4-5 hours, then cells were washed one time with serum-free Seahorse Assay Medium and incubated in Seahorse Assay Medium containing 5% FBS, 10 mM Glucose and 2 mM Glutammine in 37° C. incubator without CO₂ for 60 min. ECAR and OCR was measured under basal conditions, after treatment with TLR ligands (LPS 1 μg/ml; P3C 1 μg/ml; R848 1 μg/ml) or inhibitors (and their combination). In the assays which required pre-treatment with inhibitors, chemical inhibitors were injected into the wells by the Seahorse Analyzer, and the incubation time were allowed to proceed for 45 min, prior to the injection of TLR ligands. Triciribine (20 μM), 2-DG (25 mM), BX795 (5 μM) MRT67307 (2.5 μM), Actinomycin D (1.5 μg/ml). Data represent mean±SEM of triplicate wells. Shown is one representative experiment out of three independent experiments.

Statistical analysis. Statistical significance for experiments with more than two groups was tested with One way Anova and Tukey multiple comparison tests were performed. When comparisons between only two variables were made, unpaired two tailed t-test was used to assess statistical significance.

Adjusted p-values were calculated with Prism7 (Graphpad) or with Excel. Asterisk coding, also indicated in figure legends, is depicted as follows: *: P<=0.05; **: P<=0.01; ***: P<=0,001; ****: P<=0.0001. Data presented are representative of at least 3 independent repeats unless otherwise designated. Data with error bars are represented as mean±SEM.

Results Myddosome formation and TBK1-dependent glycolysis are commonly induced by TLR ligands. Genetically, the MyD88 signaling axis in the TLR pathway consists of a receptor (TLR), adaptors proteins (MyD88 and TIRAP) and a variety of downstream effector molecules (the IRAK family kinases, the E3 ligase TRAF6, the TAK1 complex, and IKK family kinases) (Pandey et al., 2014). These proteins collectively drive the activation of inflammatory transcriptional factors, yet their organization within the cell during signal transduction is unclear it was sought to determine whether the assembly of the myddosome initiates at the cytosolic tail of an activated TLR. We focused on the TLR4 and TLR9 pathways, as they represent prototypical receptors that signal from the cell surface and the endosomes, respectively (Pandey et al., 2014) To isolate endogenous TLR4 from immortalized bone marrow derived macrophages (iBMDMs), a biotinylated monoclonal TLR4 antibody (Sal5-21) was utilized, which interacts with TLR4 regardless of its LPS-binding state (Akashi et al., 2003). Since this monoclonal antibody could not detect denatured TLR4 (Akashi et al., 2003), a polyclonal TLR4 antibody was generated using the cytosolic tail of TLR4 as antigen, thereby enabling the detection of TLR4 by western analysis (FIG. 69A). Using this combination of reagents, it was observed that within minutes of LPS stimulation, endogenous MyD88, IRAK2, and IRAK4 were inducibly recruited to TLR4 (FIG. 69A). In contrast to TLR4, few antibody reagents are available for biochemical characterization of endogenous TLR9. Therefore, to determine the interaction between TLR9 and the myddosome, a RAW264.7 macrophage cell line expressing a C-terminally HA tagged TLR9 allele (Ewald et al., 2008) was used. Similar to the observations from the TLR4 pathway. CpG DNA stimulation led to a rapid recruitment of MyD88 and IRAK kinases to the cleaved (active) form of TLR9, as revealed by HA antibody-mediated immunoprecipitations (FIG. 69A). Importantly, the association of the myddosome components and the TLRs did not occur in unstimulated cells. Together, these results provide biochemical evidence that myddosome assembly occurs at the cytosolic tail of activated TLRs, representing one of the earliest signaling responses induced upon TLR activation.

A similar approach was taken to identify MyD88-dependent signaling components within the myddosome. We stimulated primary BM-IDMs or iBMDMs with a broader panel of TLR ligands (LPS-TLR4, P3C-TLR2, R848-TLR7), and isolated endogenous myddosomes by MyD88-immunoprecipitations. Western analysis demonstrated that, similar to IRAK2 and IRAK4, the E3 ligase TRAF6 was inducibly recruited to the myddosome upon TLR activation (FIG. 69B). Of note, the recruitment of TRAF6 was specific, as it could be only detected in MYD88 immunoprecipitates from LPS stimulated cells, but not in the IgG control immunoprecipitates (FIG. 74A) In contrast to IRAK2, 4 and TRAF6, recruitment of TAK1, IKKβ and NEMO to the myddosome was not observe under the same experimental conditions (FIG. 74A). To further validate TRAF6 as a component of the myddosome, iBMDMs were transduced with a 3×FLAG-tagged TRAF6 allele and stimulated these cells with TLR ligands. Isolation of 3×FLAG-TRAF6 via an FLAG antibody (M2) revealed that essential myddosome components (MyD88, IRAK4, and IRA1K2) were detected in the TRAF6 immunoprecipitates in a TLR-dependent manner (FIG. 69C). Again, the interactions of MyD88 and IRAK kinases with 3×FLAG-TRAF6 were specific, as such interactions were not detected in the IgG immunoprecipitates (FIG. 74B). Overall, these data establish that TRAF6 is a stable component of myddosomes that are assembled by a spectrum of TLR ligands.

Within the same time frame of myddosome formation, TLRs alter the cellular metabolic state, as exemplified by the induction of glycolysis. Since the original molecular analysis of this phenomena was made in dendritic cells (DCs) (Everts et al., 2014), it was sought to determine whether TLR stimulation also promotes glycolysis in macrophages. To this end, the seahorse technology was utilized to monitor metabolism in real-time in living macrophages. Specifically, this approach allowed the measuring of glycolysis via monitoring the rate of extracellular acidification (ECAR) resulting from the release of lactate (an end product of glycolysis) into the tissue culture medium (Pelgrom et al, 2016). Consistent with prior observations made in DCs, stimulation of primary BMDMs or iBMDMs with LPS, P3C, and R848 increased ECAR rapidly, without causing discernible changes in mitochondrial oxygen consumption (OCR) (FIGS. 74C and 74D). The observed increases in ECAR truly represents glycolysis induction, as pre-treatment of primary BMDMs with 2-DG (Grossbard and Schimke, 1966), a specific inhibitor of the glycolysis regulatory factor hexokinase blocked TLR-induced ECAR increase (FIG. 69D). TLR signaling induces the expression of several genes that regulate aerobic glycolysis (Tannahill et al., 2013). However, it was found that the rapid glycolytic burst induced by TLR ligands could proceed independent of transcription, as primary BMDMs treated with actinomycin D were still able to increase ECAR (FIG. 69E). The activity of actinomycin D was verified, as the TLR-induced transcription of Il-1b and Il-6 was prevented by treatment with this drug (FIG. 74E). These data demonstrate that the rapid induction of glycolysis is an early non-transcriptional cellular response induced by TLR activation.

In DCs, LPS-dependent early glycolysis induction relies on the IKK-related kinases TBK1 and IKKF and their direct substrate AKT (Everts et al., 2014). A signaling cascade formed by these kinases activates hexokinase, the key enzyme controlling glycolysis (Everts et al., 2014). Consistent with these findings, it was observed that chemical inhibitors of TBK1/IKKF (BX795 and MRT67307) dampened ECAR increases in TLR stimulated primary BMDMs (FIG. 74F). To genetically validate these observations, it was sought to knock down TBK1 expression in Ikbke^{-/-} iBMDMs. This approach was taken for two reasons: 1) TBK1-deficiency causes embryonic lethality (Hemmi et al., 2004). 2. TBK1 and IKKF are both able to phosphorylate AKT in vitro, providing evidence that functional redundancy exists between these kinases (Ou et al., 2011; Xie et al., 2011). To this end, Ikbke^{-/-} iBMDMs were transduced with two independent short hairpin RNAs (shRNAs) targeting TBK1 (hereafter referred to as shTBK1 #1 and shTBK1 #2). Ikbke^{-/-} iBMDMs expressing a non-targeting scramble shRNA (hereafter referred to as shCTRL) were generated as control cells. Western analysis revealed that expression of TBK1-targeting shRNAs led to more than 90% depletion of the TBK1 protein from Ikbke^{-/-} iBMDMs (FIG. 69F). The loss of TBK1 was functionally verified, as TBK1 deficient cells were defective for the LPS-induced expression of the IFN-stimulated gene (ISG) Rsad2 (FIG. 69G). These data are consistent with early studies that TBK1 is essential for TLR-induced type IFN responses (Hemmi et al., 2004; Perry et al., 2004; Fitzgerald et al., 2003). Having validated the loss of TBK1 and IKKF, we assessed TLR-mediated glycolysis induction. In comparison to their WT counterparts, Ikbke^{-/-} iBMDMs harboring the shCTRL construct were partially defective for ECAR induction upon TLR ligand stimulation (FIG. 69H). Cells lacking IKKF and TBK1 displayed profound impairment of ECAR increase (FIG. 69H). TLR-mediated AKT phosphorylation in shTBK1 cells was substantially impaired, as compared to their shCTRL counterparts (FIG. 74G). Likewise, inhibition of AKT activity in primary BMDMs via the chemical inhibitor Triciribine blocked TLR-driven ECAR increase (FIG. 74H). In contrast to the effects of TBK1 and IKKF on glycolysis, the TLR-induced activation of NF-κB and MAPK kinase pathways were not affected by the absence of TBK1/IKKF (FIG. 69I). These collective findings indicate that the TBK1-AKT signaling axis is specifically dedicated to promote glycolysis downstream of multiple TLRs. Thus, while TBK1 is most commonly discussed in the context of IRF3-mediated IFN expression (Fitzgerald et al., 2003; Hemmi et al., 2004; Perry et al., 2004), these data provide evidence that the most common function of this kinase in TLR signaling is to drive glycolysis via AKT.

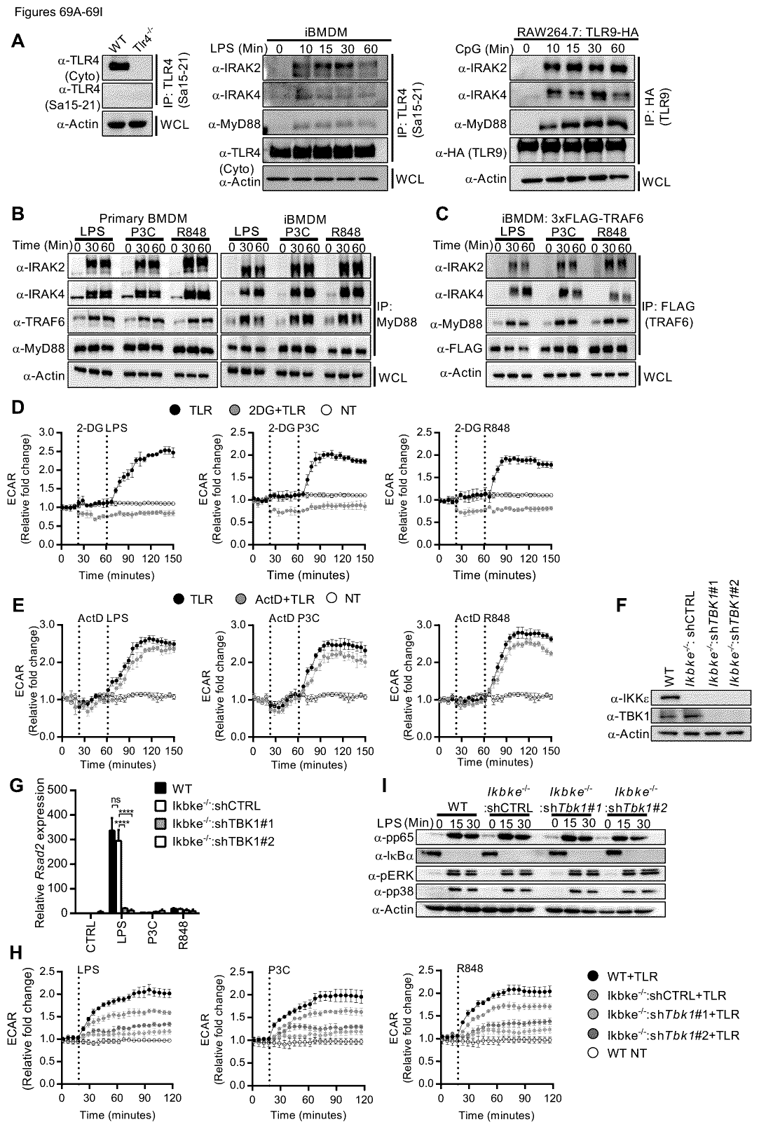

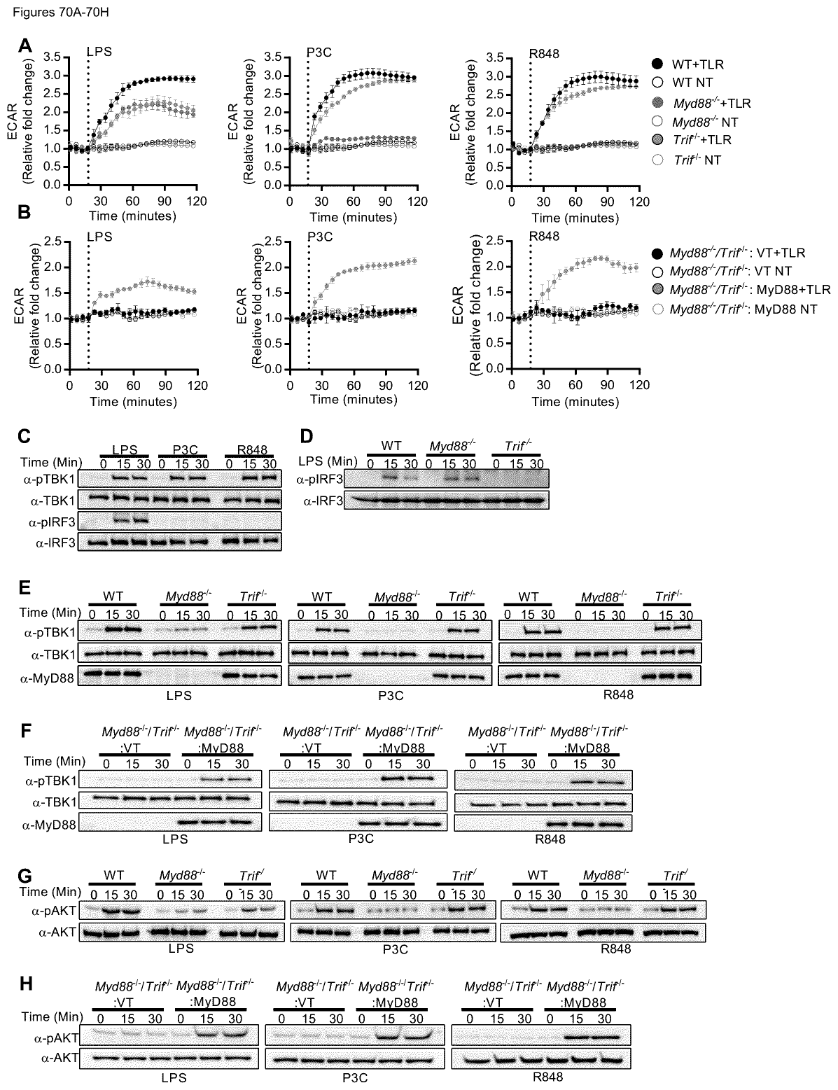

MyD88 signaling promotes TLR-induced early glycolytic burst and TBK1 activation. Whereas MyD88 and TRIF are crucial to TLR-mediated inflammatory transcriptional programs (Pandey et al., 2014), the role of these proteins in promoting TLR-mediated metabolic reprogramming is unclear. In fact, the initial identification of TBK1 as a regulator of LPS-induced glycolysis prompted speculation that this process is driven by the TRIF pathway downstream of TLR4 (O'Neill et al., 2016; Everts et al., 2014). To determine the relative contribution of MyD88 and TRIF to TLR-induced glycolysis, a genetic approach was adopted by measuring ECAR from primary WT, Myd88^{-/-}, and Trif^{-/-} BMDMs treated with TLR ligands. LPS stimulation induced robust ECAR increase in WT BMDMs, whereas ECAR increases were reduced in either Myd88^{-/-} or Trif^{-/-} BMDMs (FIG. 70A). MyD88 deficiency abolished the increase in glycolysis upon P3C and R848 stimulation (FIG. 70A). Conversely, these ligands triggered comparable ECAR increase in WT and Trif^{-/-} BMDMs (FIG. 70A). Therefore, in contrast to TRIF, MyD88 is necessary for optimal glycolytic activation in all TLR pathways examined. To corroborate these observations made in primary BMDMs, Myd88^{-/-} Trf^{-/-} iBMDMs were complemented, which are deficient for signaling downstream of all TLRs, with WT MyD88 via retroviral transduction. Cells expressing an empty retroviral vector were used as controls. This approach allowed the determination of the contribution of MyD88 to TLR-mediated early glycolysis from an isogenic background of cells. Myd88^{-/-}/Trif^{-/-} iBMDMs expressing empty vector were completely defective for ECAR induction in response to LPS, P3C or R848 (FIG. 70B) Rescuing MyD88 expression in Myd88^{-/-}/Trif^{-/-} cells restored ECAR increases upon stimulation with all TLR ligands examined (FIG. 70B). The observation that the glycolytic defects of Myd88^{-/-}/Trif^{-/-} iBMDMs can be complemented by the expression of MyD88 provides genetic proof of its critical role in promoting TLR-dependent metabolic reprogramming. Therefore, similar to TBK1, MyD88 is a common regulator of early glycolysis induced by TLR ligands in macrophages.

The finding herein, that TBK1 and MyD88 promote early glycolysis in response to TLR ligands led to a hypothesis that TBK1 could also be activated via MyD88-dependent signaling. Consistent with this idea, LPS stimulation of primary BMDMs activated TBK1 and the IFN-inducing transcription factor IRF3, whereas P3C and R848 activated TBK1, but not IRF3 (FIG. 70C). TRIF-deficiency completely abolished IRF3 phosphorylation upon LPS stimulation (FIG. 70D). Myd88^{-/-} BMDMs displayed no defects in IRF3 phosphorylation (FIG. 70D). To assess the regulation of TBK1 activation by MyD88 and TRIF, WT, Myd88^{-/-}, and Trif^{-/-} BMDMs were stimulated with LPS, P3C, and R848. Interestingly, it was observed that MyD88, rather than TRIF, primarily promoted TBK1 phosphorylation upon stimulation with TLR ligands (FIG. 70E). In comparison to their WT counterparts, LPS-induced TBK1 activation was severely impaired in Myd88^{-/-} BMDMs, but was only moderately reduced in Trif^{-/-} BMDMs (FIG. 70E). Conversely, TBK1 activation by P3C and R848 was abolished in Myd88^{-/-} BMDMs. These ligands induced equally robust TBK1 phosphorylation in WT and Trif BMDMs (FIG. 70E) Consistent with these findings, expression of MyD88 in Myd88^{-/-}/Trif^{-/-} iBMDMs restored TBK1 activation by TLR ligands, as compared to the empty vector-expressing control cells (FIG. 70F). These data therefore indicate that MyD88 is the primary driver of TBK1 activation downstream of TLR signaling.

It was then reasoned that because TBK1 is an upstream kinase of AKT, and MyD88 drives TBK1 phosphorylation, then the MyD88-TBK1 signaling axis could phosphorylate AKT to fuel glycolysis. Consistent with this idea, LPS induced the phosphorylation of AKT in primary WT BMDMs (FIG. 70G). MyD88 deficiency or TRIF deficiency diminished LPS-induced AKT phosphorylation (FIG. 70G). When stimulated with P3C and R848, AKT phosphorylation was abolished in Myd88^{-/-} cells (FIG. 70G). In contrast, these ligands induced AKT phosphorylation to a comparable extent in WT and Trif^{-/-} BMDMs (FIG. 70G). The defect in AKT phosphorylation could be complemented by retroviral expression of MyD88 in Myd88^{-/-}/Trif^{-/-} iBMDMs (FIG. 70H). The pattern of AKT phosphorylation in LPS, P3C, and R848-stimulated cells therefore follows a similar trend to that of TBK1 phosphorylation. In summary, these data provide biochemical validation to our metabolic analysis, in that a newly-defined “MyD88-TBK1-AKT” signaling axis primarily promotes early glycolysis in response to a broad spectrum of TLR ligands.

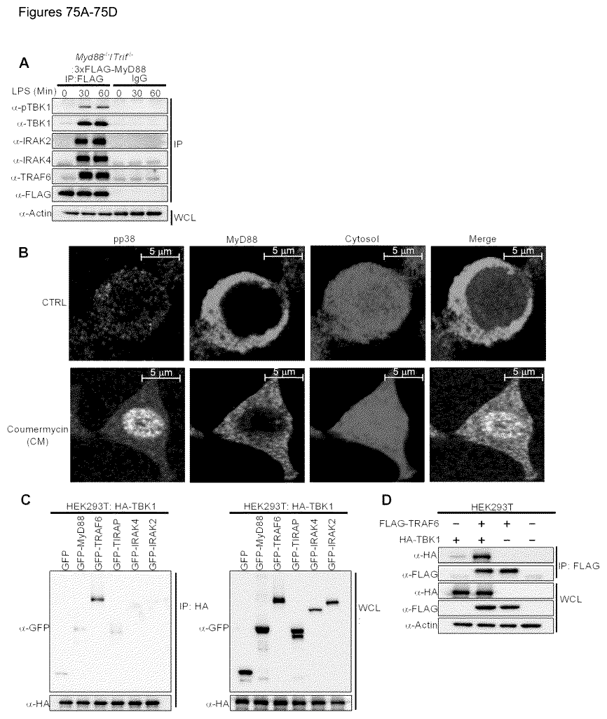

TBK1 is a novel component of the myddosome. While the data indicate that MyD88 is genetically required for TBK1 activation, the mechanism by which TBK1 is activated by MyD88 signaling remains undefined. Since the myddosome has been proposed to function as a SMOC to propagate MyD88-signaling upon TLR activation, it was sought to determine whether TBK1 is “locally” activated within the myddosome by being a component of this complex. Alternatively, this kinase may be activated downstream of myddosome activity, within a distinct protein complex. To this end, WT iBMDMs were stimulated with LPS, P3C, and R848 to induce myddosome formation. It was observed that TBK1 was recruited to MyD88 immunoprecipitates in TLR-stimulated cells (FIG. 71A). Furthermore, the myddosome-associated TBK1 was active, as revealed by western analysis using a phospho-specific antibody raised against TBK1 (FIG. 71A). Because both MyD88 and TRIF can mediate TBK1 activation upon LPS stimulation, it is possible that TRIF signaling might facilitate the recruitment of TBK1 to the myddosome. To address this possibility, a 3×FLAG-tagged MyD88 allele was introduced in Myd88^{-/-}/Trif^{-/-} iBMDMs via retroviral transduction. FLAG-specific antibody (M2) immunoprecipitates from LPS. P3C, and R848-stimulated cells contained the myddosome components IRAK2, IRAK4, and TRAF6, as well as total TBK1 and phosphorylated TBK1 (FIG. 71B). The recruitment of TBK1 to the myddosome was specific, as no such recruitment could be detected in IgG immunoprecipitates (FIG. 75A). As these experiments were performed in cells that lack TRIF, they eliminate the possibility that TRIF-mediated TBK1 activation facilitates the association of this kinase with the myddosome.

To corroborate these biochemical analyses, it was determined if phosphorylated TBK1 could be detected within myddosomes within intact cells. In order to facilitate this cell biological analysis, it was sought to synchronize myddosome formation. To this end, an approach was used (Hacker et al., 2006), where a GyrB domain (from the Escherichia coli DNA gyrase) was fused to the C terminus of a 3×FLAG-MyD88 allele (hereafter referred to as 3×FLAG-MyD88-GyrB). This allele enabled the chemical induction of the entire population of MyD88 to assemble into myddosomes via the compound coumermycin (CM). To validate the functionality of this construct, Myd88^{-/-}/Trif^{-/-} iBMDMs stably transduced with the 3×FLAG-MyD88-GyrB allele were stimulated with CM, LPS, and P3C. CM treatment induced myddosome formation and Il-1b expression to an extent comparable to that induced by LPS and P3C treatment (FIGS. 71C and 71D), thereby establishing these cells as a suitable model to study myddosome formation Using immunofluorescence, the staining patterns of endogenous phosphorylated TBK1 (pTBK1) and MyD88 was examined in cells stimulated with CM. In untreated cells, MyD88 staining was scattered throughout the cytoplasm (FIG. 71E), whereas pTBK1 staining appeared to be dim or non-specific, as this kinase is inactive at steady state (FIG. 71E). Notably, CM-treatment induced the formation of distinct “MyD88 specks” resembling the cellular sites of myddosome formation (in more than 60% cells examined) (FIGS. 71E and 71F). Notably, MyD88 specks stained positive for pTBK1 (FIGS. 71E and 71F), indicating that the active kinase was concentrated within these structures. On the contrary, phosphorylated p38 (pp38), which is also activated by MyD88, was not detected within MyD88 specks in CM-stimulated cells (FIG. 75B). This kinase was clearly activated by CM, as indicated by its expected nuclear staining pattern (Gong et al., 2010) (FIG. 75B). These collective biochemical and microscopic observations therefore support the conclusion that TBK1 is recruited to and activated within the myddosome.

To determine the mechanism by which TBK1 is recruited to the myddosome, the association between TBK1 and individual myddosome components was examined using 293T cells as a model. Plasmids encoding HA-tagged TBK1 and GFP-tagged myddosome components (MyD88, TIRAP, IRAK2, IRAK4, and TRAF6) were transiently transfected in 293T cells in a pairwise manner. Western analysis of HA antibody-mediated immunopreciptations revealed that TRAF6 associated with TBK1, as compared to the empty vector control (FIG. 75C). Other myddosome components did not form a complex with TBK1 (FIG. 75C). Reciprocally, TRAF6 was isolated from 293T cells transfected with FLAG-tagged TRAF6 and HA-tagged TBK1 via the M2 FLAG antibody. Western analysis revealed that TBK1 could also be detected in TRAF6 immunoprecipitates (FIG. 75D). Together, these data suggest that TRAF6 could form a complex with TBK1.

It was reasoned that if TRAF6 bridges TBK1 to the myddosome, then reducing TRAF6 expression should impair the recruitment of TBK1 to this SMOC. To test this possibility, a pair of RAW264.7 cell lines stably expressing a TRAF6-targeting shRNA (shTRAF6) and a scramble shRNA control (shCTRL) were utilized (West et al., 2011). Western analysis demonstrated the significant reduction of TRAF6 protein abundance in shTRAF6 expressing cells (FIG. 71G). Consistent with the essential role of TRAF6 in promoting NF-KB activation (Deng et al., 2000), LPS, P3C, and R848 induced Il-1b and Il-6 expression was impaired in shTRAF6 cells in comparison to shCTRL cells (FIG. 71H). Interestingly, shTRAF6 expression did not cause major changes in the recruitment of IRAK kinases to MyD88 immunoprecipitates (FIG. 71I), indicating that TRAF6 is not necessary for myddosome assembly. In contrast, TRAF6 was required for recruitment of total and active TBK1 into myddosomes, as compared to the shCTRL cells (FIG. 71I). This diminished recruitment of TBK1 into myddosomes corresponded to diminished TBK1 phosphorylation in lysates from cells stimulated by TLR ligands (FIG. 71G). TRAF6 is therefore not required for myddosome assembly, but rather mediates the recruitment and activation of TBK1 within the myddosome. These findings provide insight into the mechanisms by which the effector functions of the myddosome are regulated.

Synthetic myddosomes can be engineered to induce type I IFN responses and RIP3-dependent necroptosis upon TLR activation. While the experiments described above establish the myddosome as a multifunctional signaling organelle, the activities that were examined have been selected by evolution. It was reasoned that if the myddosome is indeed a modular signaling organelle, then it should be amenable to synthetic engineering to entice novel signaling outcomes. The discovery herein, of TBK1 as a novel component of the myddosome provides a unique opportunity to test this idea.

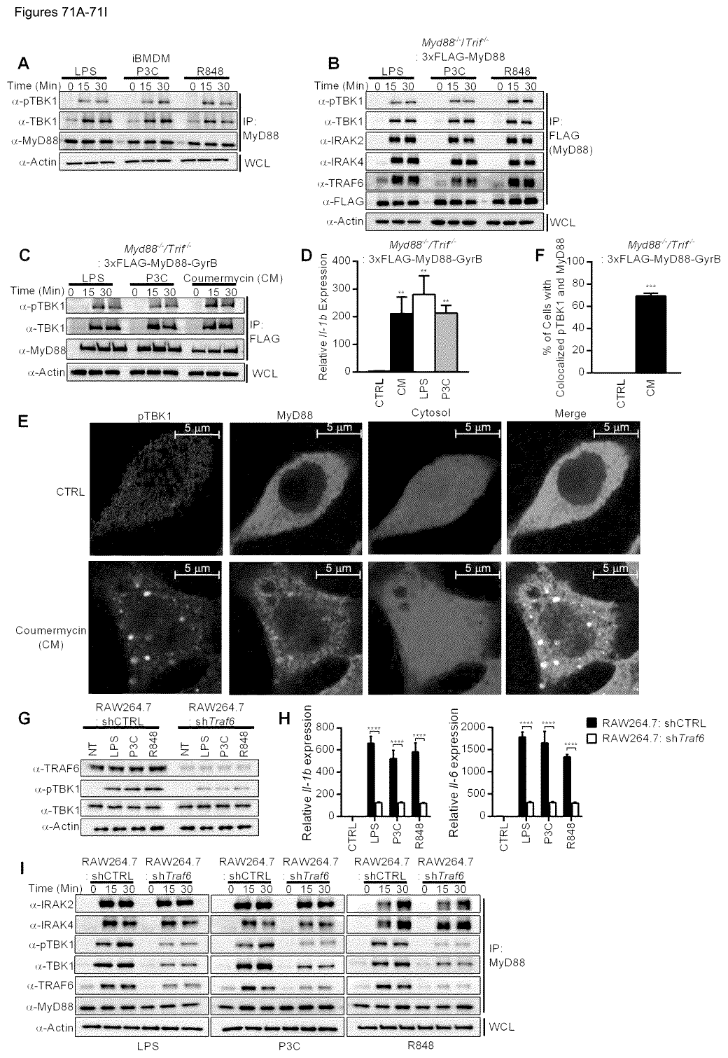

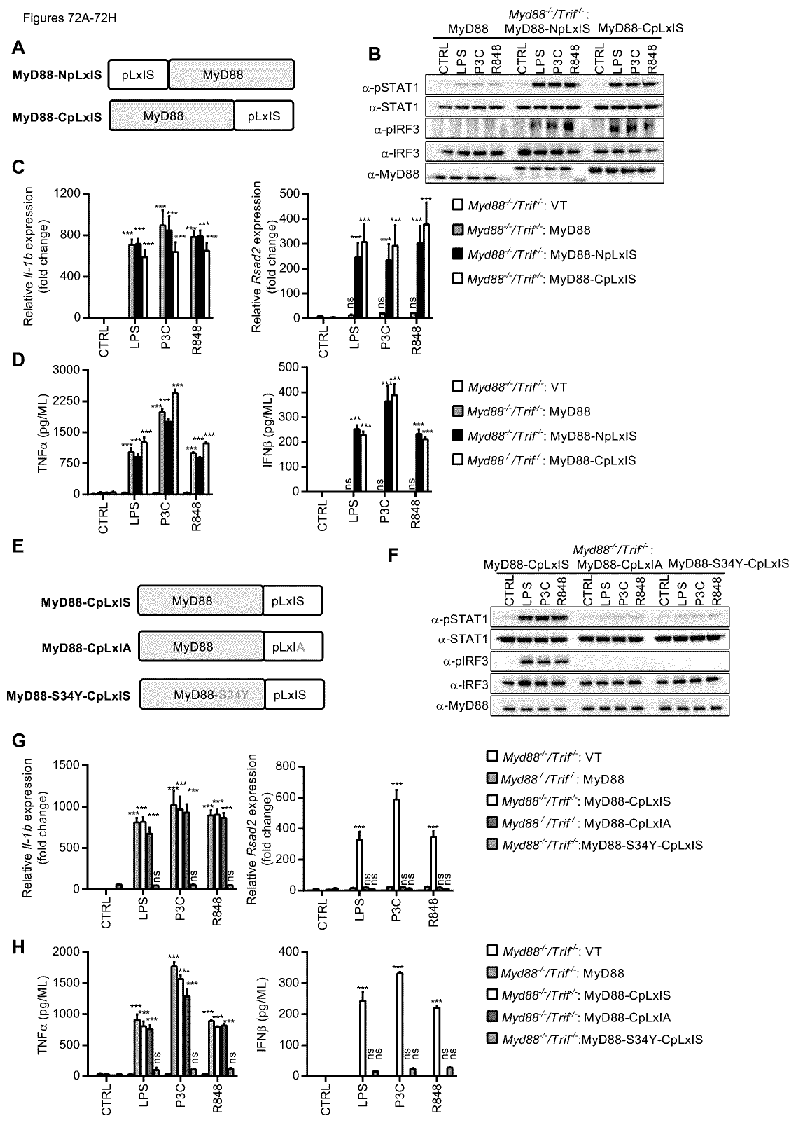

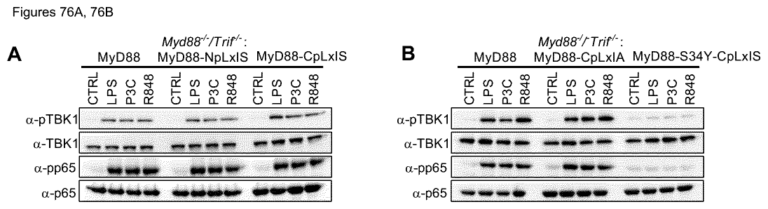

Functionally analogous to MyD88, adaptor proteins are used by most PRR pathways to mediate signal transduction: TRIF is the analogous signaling adaptor for the TLR3/4 pathways, MAVS for the RLR pathway, and STING for the cGAS pathway (Brubaker et al., 2015). Interestingly, TRIF, MAVS, and STING all activate TBK1 to promote IRF3 phosphorylation and type I IFN expression (Liu et al., 2015). In contrast, MyD88 signaling activates TBK1 without activating IRF3 or IFN expression. Notably, TRIF, STING, and MAVS all share a “pLxIS” motif (p, hydrophilic residue; x, any residue; S, phosphorylation site) (Liu et al., 2015). This motif is necessary for these adaptors to link TBK1 to the activation of IRF3. MyD88 does not contain a pLxIS motif. This disparity raised the question of whether the myddosome could be rewired to drive IFN expression and signaling. It was reasoned that if the myddosome is truly a modular organizing center, then synthetic immunology-based approaches could be used to generate MyD88-pLxIS chimeric alleles that endow the myddosome with the ability to activate IRF3. To test this idea, MyD88-pLxIS chimera alleles were generated by fusing the pLxIS motif from STING to either the N-terminus or the C-terminus of MyD88 (hereafter referred to as MyD88-NpLxIS and MyD88-CpLxIS) (FIG. 72A). These constructs were retrovirally 25 transduced into Myd88^{-/-}/Trif^{-/-} iBMDMs, which permitted analysis of their signaling properties at multiple levels. In particular, NF-κB p65 phosphorylation, Il-1b gene expression, and TNFα secretion were used as readouts of pro-inflammatory cytokine signaling. IRF3 and STAT1 phosphorylation, Rsad2 expression, and IFNβ secretion were chosen as readouts for type I IFN responses. TBK1 phosphorylation was also monitored, because of its importance in activating the pLxIS motif. Introduction of WT MyD88 into Myd88^{-/-}/Trif^{-/-} iBMDMs restored phosphorylation of TBK1 and p65, Il-1b gene expression, and TNFα secretion (FIGS. 72C-72D and 76A). No detectable changes in IRF3 or STAT1 activation, Rsad2 expression or IFNβ release were observed in cells expressing WT MyD88 (FIGS. 72B-72D). These observations are consistent with the role of MyD88 in promoting pro-inflammatory cytokine, rather than type I IFN expression, in macrophages. Expression of the MyD88-NpLxIS or the MyD88-CpLxIS alleles in Myd88^{-/-}/Trif^{-/-} iBMDMs complemented the defects in pro-inflammatory cytokine signaling to an extent similar to their WT MyD88-expressing counterparts (FIGS. 72C-72D and 76A). Strikingly, these two synthetic MyD88 alleles activated type I IFN signaling upon TLR stimulation, as demonstrated by IRF3 and STAT1 phosphorylation, Rsad2 gene expression, and IFNβ release (FIGS. 72B-72D). Of note, the protein expression levels of these MyD88 alleles were comparable (FIG. 72B). These data demonstrate that the synthetic MyD88-NpLxIS and MyD88-CpLxIS alleles are functionally capable of activating type I IFN responses, thereby expanding the transcriptional circuits controlled by the myddosome.

The molecular mechanism by which these synthetic MyD88 alleles activated type I IFN expression was then determined. Since differences in signaling capacity between the MyD88-NpLxIS and the MyD88-CpLxIS alleles were not observed, the MyD88-CpLxIS allele was chosen for mechanistic studies. The focus was on two mutants: the first mutant is MyD88-CpLxIA, which abolishes the Ser residue critical for IRF3 activation upon TBK1 phosphorylation (Liu et al., 2015, Tanaka and Chen, 2012). The second mutant is MyD88-S34Y-CpLxIS, which contains an intact pLxIS motif but impairs MyD88 oligomerization (George et al., 2011, Nagpal et al, 2011) (FIG. 72E). It was predicted that if the IFN-inducing synthetic myddosome signals in a modular manner, then abolishing the IRF3 activation motif should only impair the type I IFN inducing activity emanating from this complex, while leaving the pro-inflammatory signaling intact It was further predicted that if oligomerization is truly key to SMOC signaling, as suggested by structural analysis, then blocking MyD88 oligomerization should dampen both signaling outcomes.

Consistent with these predictions, cells expressing the MyD88-CpLxIS and the MyD88-CpLxIA alleles displayed comparable levels of TBK1 and p65 phosphorylation, Il-1b gene expression, and TNFα production (FIGS. 72G-72H and 76B). In contrast, IRF3 and STAT1 phosphorylation, Rsad2 expression, and IFNβ secretion were all abolished in MyD88-CpLxIA expressing cells, as compared to WT counterparts (FIGS. 72F-72H). Cells harboring the oligomerization-deficient allele, MyD88-S34Y-CpLxIS, were poorly responsive to TLR ligands, which was revealed by their substantial defects in pro-inflammatory cytokine and type I IFN signaling (FIGS. 72F-72H and 76B). Importantly, the expression levels of distinct MyD88 alleles were comparable among these stable lines (FIG. 72F). These results therefore provide mechanistic insights into the rewired transcriptional circuits triggered by the synthetic MyD88-pLxIS molecule. Specifically, upon TLR-induced myddosome oligomerization, the pLxIS motif-containing MyD88 recruits and activates TBK1, which in turn utilizes the pLxIS motif to activate IRF3.

To determine whether the myddosome could be reprogrammed to induce cellular processes beyond orchestrating distinct transcriptional programs, the focus was on inflammatory cell death. One form of inflammatory cell death is necroptosis (Galluzzi et al, 2017; Moriwaki and Chan, 2013), which is executed by the actions of RIP family kinases (RIP1 and RIP3), and mixed lineage kinase domain-like protein (MLKL) Mechanistically, RIP1 and RIP3 form an oligomeric complex, which then recruits and phosphorylates MLKL, the executor of necroptosis (Li et al., 2012; Sun et al., 2012; Wang et al., 2014). Importantly, TLR signaling via MyD88 does not directly activate necroptosis (Kaiser et al, 2013).

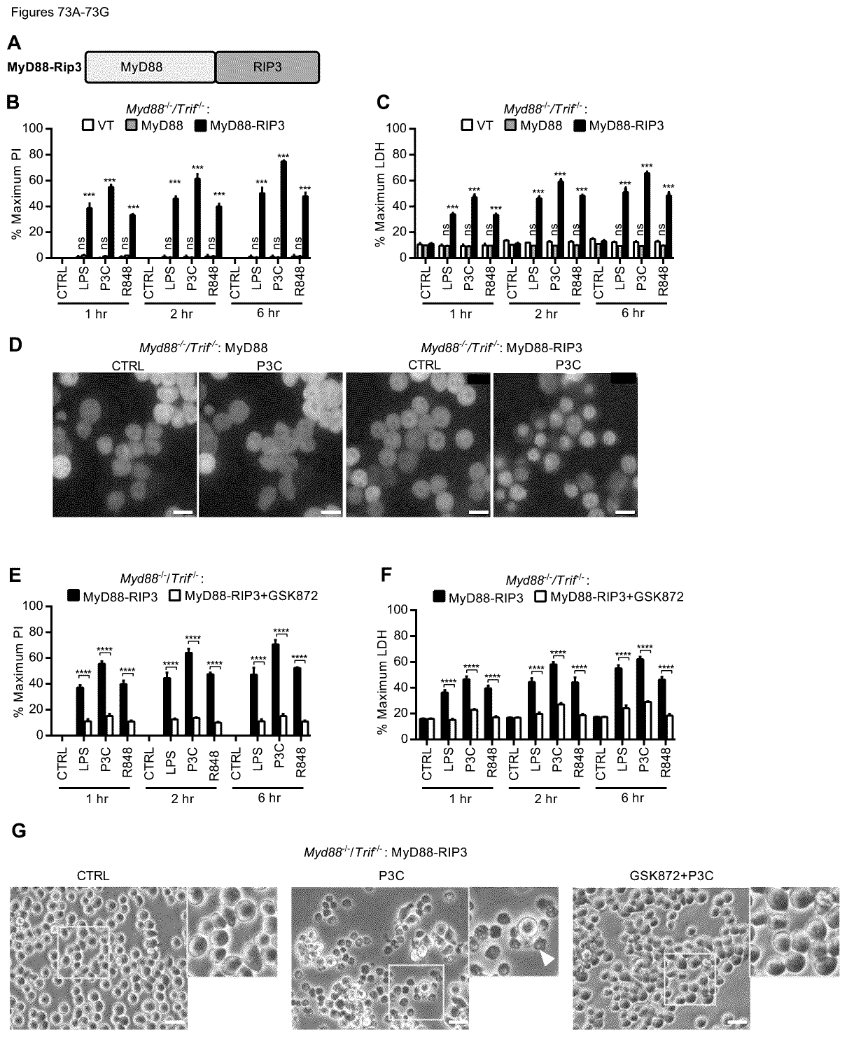

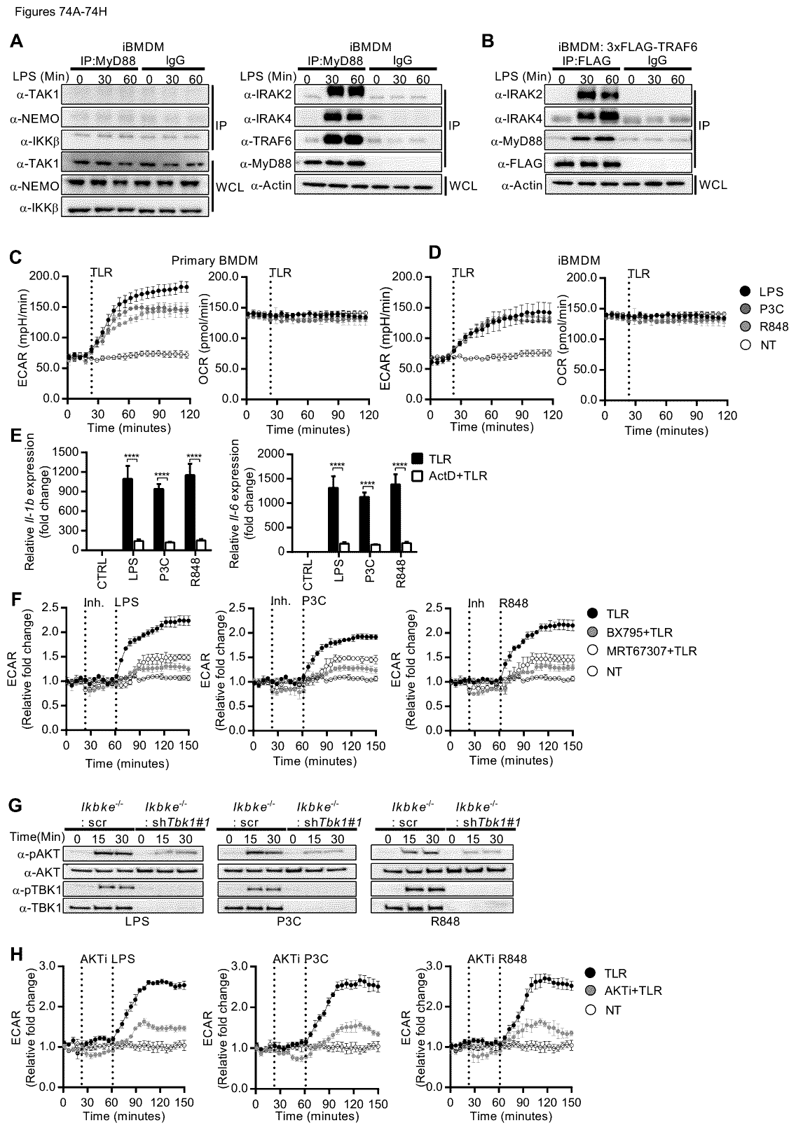

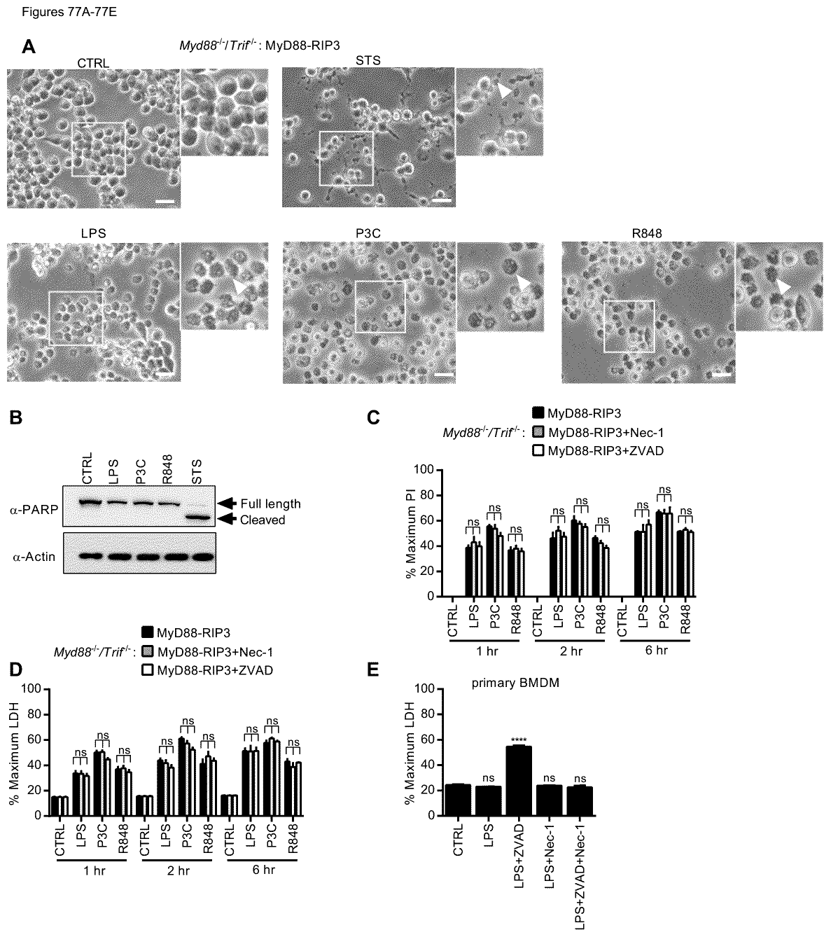

Based on the premise stated above, it was sought to determine whether MyD88 signaling could be reprogrammed to directly activate necroptosis by generating an MyD88-RIP3 fusion allele (FIG. 73A). It was reasoned that because oligomerization is a shared mechanism between necroptosis initiation and myddosome signaling, then the MyD88-RIP3 allele should be able to directly promote necroptosis To test this hypothesis, Myd88′Trif-iBMDMs stably expressing WT MyD88, MyD88-RIP3, and an empty vector (as control) were generated. As compared to the non-inflammatory cell death program-apoptosis, hallmarks of necroptosis include the loss of plasma-membrane integrity and the release of the cytosolic enzyme lactase dehydrogenase (LDH) (Galluzzi et al., 2017; Wang et al., 2014). The loss of membrane integrity allows for the labeling of intracellular nucleic acids by propidium iodide (PI), a membrane impermeable compound. Therefore, PI staining and LDH release was used to measure TLR-induced necroptosis from these cell lines. Myd88^{-/-}/Trif^{-/-} iBMDM expressing an empty retroviral vector did not stain with PI or release LDH after TLR stimulation (FIGS. 73B and 73C). Cells expressing WT MyD88 also did not stain with PI or release LDH (FIGS. 73B-73D), as expected. In contrast, TLR stimulation led to rapid PI staining and LDH release from cells harboring the MyD88-RIP3 allele, as both markers became readily detectable within the first hour of ligand stimulation (FIGS. 73B-73D). This process is dependent on RIP3 activity, because GSK872 (Kaiser et al., 2013), a chemical inhibitor of RIP3, blocked PI staining and LDH release from MyD88-RIP3 expressing cells in the presence of TLR ligands (FIGS. 73E-73F). Moreover, visual examination of TLR-stimulated cells revealed that cells harboring the MyD88-RIP3 allele displayed morphological features indicative of cell lysis (e.g. phase dense shrunken cell corpses) (FIG. 73G). Such morphological changes could be suppressed by pre-treatment of GSK872 (FIG. 73G). The morphological changes resulted from TLR-mediated MyD88-RIP3 activation were distinct from those induced by the apoptosis-inducing agent staurosporine (e.g. membrane blebbing, generation of apoptotic bodies/vesicles) (Galluzzi et al., 2012) (FIG. 77A). Furthermore, staurosporine treatment induced PARP cleavage, a molecular marker of apoptosis (Green and Kroemer, 1998) (FIG. 77B). No cleaved PARP could be detected from the MyD88-RIP3 allele-expressing cells stimulated with TLR ligands (FIG. 77B), highlighting that the cell death program triggered by this allele is distinct from apoptosis. Consistent with the caspase-independent nature of necroptosis, the pan-caspase inhibitor ZVAD failed to prevent membrane rupture and cell death induced by the MyD88-RIP3 allele upon TLR activation (FIGS. 77C and 77D). Naturally, RIP3 functions downstream of RIP1 in the necroptotic pathway. Since the myddosome provides an oligomeric signaling platform to nucleate RIP3 directly, it was predicted that the execution of MyD88-RIP3-mediated cell death could bypass the requirement of RIP1. Indeed, TLR-induced PI staining and LDH release from MyD88-RIP3 expressing cells were unaffected by blockade of RIP1 activity using the inhibitor Nec-1 (FIGS. 77C and 77D). Importantly, pre-treatment of primary BMDMs with ZVAD followed by LPS stimulation induced necroptosis, which could be prevented by Nec-1 (FIG. 77E). This proof-of-concept experiment demonstrates the efficacy of the chemical inhibitors used in these assays Collectively, these data establish that the myddosome could be reprogrammed to induce RIP3-dependent necroptotic cell death.

### Discussion

In this study, experimental support was provided to the central hypothesis that the myddosome is a bonafide organizing center, as this SMOC is the subcellular site of signals that induce NF-κB activation and glycolysis. The glycolysis-inducing function of the myddosome is fulfilled by TRAF6-mediated recruitment and activation of TBK1, a kinase key to the induction of the AKT-dependent glycolytic responses (Everts et al, 2014). The emanation of these cellular processes from the myddosome therefore demonstrates the modular nature of SMOC-mediated signal transduction. Indeed, leveraging the modularity of myddosome signaling, this signaling platform was synthetically reprogramed to induce type I IFN responses and RIP3-dependent necroptosis. These findings therefore fill an important gap in the understanding of how distinct cellular processes can be coordinated by a single signaling platform upon TLR activation.

The identification herein, of TRAF6 as a core component of the myddosome is notable, because all of the previously characterized myddosome components share similar domains that allow for homotypic protein-protein interactions (Lin et al., 2010). In particular, TLRs, TIRAP, and MyD88 possess a Toll/interleukin-1 receptor homology domain (TIR) domain (Pandey et al, 2014). MyD88 and IRAK kinases contain death domains (DD) (Pandey et al., 2014). In cell-free systems, self-association of these domains drives the formation of higher-ordered helical structures (Lin et al., 2010; Ve et al., 2017). However, TRAF6 does not harbor a DD or a TIR domain. These results provide evidence that within cells, the myddosome is not merely a stack of proteins containing TIRs or DDs that only activates pro-inflammatory transcription programs. Indeed, it was revealed herein that TRAF6-mediated TBK1 recruitment and activation within the myddosome. This finding elucidates how the myddosome promotes early glycolysis upon microbial encounters. A further implication of this discovery is that additional host proteins (non-TIR-, non-DD-containing) might be recruited to the myddosome upon microbial sensing to promote distinct signaling processes. However, it is noteworthy that additional questions remain. For instance, the biochemical interactions among the endogenous myddosome, TAK-1 signaling complex, and the IKK complex in professional phagocytes warrant further exploration. Furthermore, whereas the E3 ubiquitin ligase activity of TRAF6 is well-established to generate K63 linked ubiquitin chains for TAK1 activation (Deng et al., 2000; Xia et al., 2009), emerging evidence indicates that this protein carries E3 ligase-independent functions (Strickson et al., 2017). A potential scaffolding function of TRAF6, and the functional redundancy between TRAF6 and other host factors, must therefore be explored.

A central finding of this study is the presence of TBK1 within the myddosome. Shared by multiple anti-viral PRR signaling pathways, TBK1 is well-recognized for its role in inducing type I IFN responses by activating IRF3 (Liu et al., 2015). Emerging evidence has also highlighted activities of TBK1 that are distinct from IRF3 For example, in addition to promoting glycolysis in DCs, this kinase fine tunes the activation state of other IKK members and may influence inflammatory transcription programs (Clark et al, 2011; Hacker et al., 2006). These studies, however, did not explore the upstream source of signals that induce TBK1 activation. It is this latter point that most distinguishes the work herein from others, as the myddosome was identified as a site of TBK1 recruitment and activation. Mechanistically, it was found that, upon recruitment by TRAF6, TBK1 is activated within the myddosome, which in turn promotes AKT phosphorylation to drive TLR-dependent early glycolysis. This finding not only provides mechanistic insight into MyD88-dependent TBK1 activation, but also expands the physiological functions the myddosome (from a regulator of transcription to a regulator to metabolic reprogramming). The early glycolytic induction mediated by the “MyD88-TBK1-AKT” signaling axis is just the beginning of profound host metabolic alterations induced by PRR signaling (O'Neill et al., 2016) Indeed, AKT, the master regulator of metabolism, is also regulated by divergent host factors which include but not limited to PI3K, MTOR, and BCAP (Huang et al., 2016; Krawczyk et al., 2010; Troutman et al., 2012). Many of these factors facilitate the long-term commitment of professional phagocytes to glycolysis (O'Neill et al., 2016), and likely act downstream of the immediately acting cellular responses that were described herein. These findings, coupled with these data, therefore emphasize the importance of understanding how the myddosome coordinates short-term and long-term metabolic needs upon TLR activation.

The observations that the myddosome organizes pro-inflammatory transcriptional programs and metabolic reprogramming highlights the modular nature of myddosome signaling, underpinning the proposal that SMOCs function as organizing centers in PRR signaling. Yet, is it possible that the modularity of myddosome signaling only governs the operation of naturally occurring cellular processes shaped by evolution? Or is it possible that the modularity of SMOC signaling would enable the reprograming of signaling outcomes by synthetic engineering of novel signaling circuits? The data herein, using synthetic myddosomes that promote type I IFN expression and RIP3 mediated necroptosis provide an affirmative answer to the latter scenario. These findings are therefore consistent with a theory in evolutionary biology that functional diversity in signaling pathways could evolve through the recombination of motifs/domains (Carroll, 2008). In other words, modularity often equates to evolvability.

The findings herein show that synthetic immunology-based approaches can be used to engineer unique and beneficial cellular responses. Indeed, these approaches may be considered akin to those taken to rewire signaling pathways in lymphocytes to generate chimeric antigen receptor (CAR) T cell therapies in the clinic.

In summary, the herein data support the hypothesis that unifying themes exist to explain the operation of the diverse signaling proteins and pathways in the innate immune system One such theme is that effector function diversity can be achieved by the use of a single organizing center. The discoveries presented here therefore provide a mandate to explore the natural and potentially programmable features of other signaling organelles that diversify activities within the innate immune system.

### REFERENCES

Akashi, S., Saitoh, S., Wakabayashi, Y., Kikuchi, T., Takamura, N., Nagai, Y., Kusumoto, Y., Fukase, K., Kusumoto, S., Adachi, Y., et al. (2003). Lipopolysaccharide interaction with cell surface Toll-like receptor 4-MD-2: higher affinity than that with MD-2 or CD14. J Exp Med 198, 1035-1042.
Bonham, K. S., Orzalli, M. H., Hayashi, K., Wolf, A. I., Glanemann, C., Weninger, W., Iwasaki, A., Knipe, D. M., and Kagan, J. C. (2014). A promiscuous lipid-binding protein diversifies the subcellular sites of toll-like receptor signal transduction. Cell 156, 705-716.
Brubaker, S. W., Bonham, K. S., Zanoni, I., and Kagan, J. C. (2015). Innate immune pattern recognition: a cell biological perspective. Annu Rev Immunol 33, 257-290.
Cai, X., Chen, J., Xu, H., Liu, S., Jiang, Q. X., Halfmann, R., and Chen, Z. J. (2014). Prion-like polymerization underlies signal transduction in antiviral immune defense and inflammasome activation. Cell 156, 1207-1222.
Carroll, S. B. (2008). Evo-devo and an expanding evolutionary synthesis: a genetic theory of morphological evolution. Cell 134, 25-36.
Clark, K., Takeuchi, O., Akira, S., and Cohen, P. (2011). The TRAF-associated protein TANK facilitates cross-talk within the IkappaB kinase family during Toll-like receptor signaling. Proc Natl Acad Sci USA 108, 17093-17098.
Deng, L., Wang, C., Spencer, E., Yang, L., Braun, A., You, J., Slaughter, C., Pickart, C., and Chen, Z. J. (2000). Activation of the IkappaB kinase complex by TRAF6 requires a dimeric ubiquitin-conjugating enzyme complex and a unique polyubiquitin chain. Cell 103, 351-361.
Everts, B., Amiel, E., Huang, S. C., Smith, A. M., Chang, C. H., Lam, W. Y., Redmann, V., Freitas, T. C., Blagih, J., van der Windt, G. J., et al. (2014). TLR-driven early glycolytic reprogramming via the kinases TBK1-IKKvarepsilon supports the anabolic demands of dendritic cell activation. Nat Immunol 15, 323-332.
Ewald, S. E., Lee, B. L., Lau, L., Wickliffe, K. E., Shi, G. P., Chapman, H. A., and Barton, G. M. (2008). The ectodomain of Toll-like receptor 9 is cleaved to generate a functional receptor. Nature 456, 658-662.
Fitzgerald, K. A., McWhirter, S. M., Faia, K. L., Rowe, D. C., Latz, E., Golenbock, D. T., Coyle, A. J., Liao, S. M., and Maniatis, T. (2003). IKKepsilon and TBK1 are essential components of the IRF3 signaling pathway. Nat Immunol 4, 491-496.
Galluzzi, L., Kepp, O., Chan, F. K., and Kroemer, G. (2017). Necroptosis: Mechanisms and Relevance to Disease. Annu Rev Pathol 12, 103-130.
Galluzzi, L., Vitale, I., Abrams, J. M., Alnemri, E. S., Baehrecke, E. H., Blagosklonny, M. V., Dawson, T. M., Dawson, V. L., El-Deiry, W. S., Fulda, S., et al. (2012). Molecular definitions of cell death subroutines: recommendations of the Nomenclature Committee on Cell Death 2012. Cell Death Differ 19, 107-120.
Gay, N. J., Gangloff, M., and O'Neill, L. A. (2011). What the Myddosome structure tells us about the initiation of innate immunity. Trends Immunol 32, 104-109.
Gay, N. J., Symmons, M. F., Gangloff, M., and Bryant, C. E. (2014). Assembly and localization of Toll-like receptor signalling complexes. Nat Rev Immunol 14, 546-558.
George, J., Motshwene, P. G., Wang, H., Kubarenko, A. V., Rautanen, A., Mills, T. C., Hill, A. V., Gay, N. J., and Weber, A. N. (2011). Two human MYD88 variants, S34Y and R98C, interfere with MyD88-IRAK4-myddosome assembly. J Biol Chem 286, 1341-1353.
Gong, X., Ming, X., Deng, P., and Jiang, Y. (2010). Mechanisms regulating the nuclear translocation of p38 MAP kinase. J Cell Biochem 110, 1420-1429.
Green, D., and Kroemer, G. (1998). The central executioners of apoptosis: caspases or mitochondria? Trends Cell Biol 8, 267-271.
Grossbard, L., and Schimke, R. T. (1966). Multiple hexokinases of rat tissues. Purification and comparison of soluble forms. J Biol Chem 241, 3546-3560.
Hacker, H., Redecke, V., Blagoev, B., Kratchmarova, I., Hsu, L. C., Wang, G. G., Kamps, M. P., Raz, E., Wagner, H., Hacker, G., et al. (2006). Specificity in Toll-like receptor signalling through distinct effector functions of TRAF3 and TRAF6. Nature 439, 204-207.
Hemmi, H., Takeuchi, O., Sato, S., Yamamoto, M., Kaisho, T., Sanjo, H., Kawai, T., Hoshino, K., Takeda, K., and Akira, S. (2004). The roles of two IkappaB kinase-related kinases in lipopolysaccharide and double stranded RNA signaling and viral infection. J Exp Med 199, 1641-1650.
Hu, Z., Zhou, Q., Zhang, C., Fan, S., Cheng, W., Zhao, Y., Shao, F., Wang, H. W., Sui, S. F., and Chai, J. (2015). Structural and biochemical basis for induced self-propagation of NLRC4. Science 350, 399-404.
Huang, S. C., Smith, A. M., Everts, B., Colonna, M., Pearce, E. L., Schilling, J. D., and Pearce, E. J. (2016). Metabolic Reprogramming Mediated by the mTORC2-IRF4 Signaling Axis Is Essential for Macrophage Alternative Activation. Immunity 45, 817-830.
Janeway, C. A., Jr. (1989). Approaching the asymptote? Evolution and revolution in immunology. Cold Spring Harb Symp Quant Biol 54 Pt 1, 1-13.
Jiang, X., Kinch, L. N., Brautigam, C. A., Chen, X., Du, F., Grishin, N. V., and Chen, Z. J. (2012). Ubiquitin-induced oligomerization of the RNA sensors RIG-I and MDA5 activates antiviral innate immune response. Immunity 36, 959-973.
Kagan, J. C., and Barton, G. M. (2015). Emerging principles governing signal transduction by pattern-recognition receptors. Cold Spring Harb Perspect Biol 7, a016253.
Kagan, J. C., Magupalli, V. G., and Wu, H. (2014). SMOCs: supramolecular organizing centres that control innate immunity. Nat Rev Immunol 14, 821-826.
Kaiser, W. J., Sridharan, H., Huang, C., Mandal, P., Upton, J. W., Gough, P. J., Sehon, C. A., Marquis, R. W., Bertin, J., and Mocarski, E. S. ( ). Toll-like receptor 3-mediated necrosis via 15 TRIF, RIP3, and MLKL. J Biol Chem 288, 31268-31279.
Krawczyk, C. M., Holowka, T., Sun, J., Blagih, J., Amiel, E., DeBerardinis, R. J., Cross, J. R., Jung, E., Thompson, C. B., Jones, R. G., et al. (2010). Toll-like receptor-induced changes in glycolytic metabolism regulate dendritic cell activation. Blood 115, 4742-4749.
Li, J., McQuade, T., Siemer, A. B., Napetschnig, J., Moriwaki, K., Hsiao, Y. S., Damko, E., Moquin, D., Walz, T., McDermott, A., et al. (2012). The RIP1/RIP3 necrosome forms a functional amyloid signaling complex required for programmed necrosis. Cell 150, 339-350.
Lin, S. C., Lo, Y. C., and Wu, H. (2010). Helical assembly in the MyD88-IRAK4-IRAK2 complex in TLR/IL-1R signalling. Nature 465, 885-890.
Liu, S., Cai, X., Wu, J., Cong, Q., Chen, X., Li, T., Du, F., Ren, J., Wu, Y. T., Grishin, N. V., et al. (2015). Phosphorylation of innate immune adaptor proteins MAVS, STING, and TRIF induces IRF3 activation. Science 347, aaa2630.
Lu, A., Magupalli, V. G., Ruan, J., Yin, Q., Atianand, M. K., Vos, M. R., Schroder, G. F., Fitzgerald, K. A., Wu, H., and Egelman, E. H. (2014). Unified polymerization mechanism for the assembly of ASC-dependent inflammasomes. Cell 156, 1193-1206.
Medzhitov, R. (2009). Approaching the asymptote: 20 years later. Immunity 30, 766-775.
Medzhitov, R., and Horng, T. (2009). Transcriptional control of the inflammatory response. Nat Rev Immunol 9, 692-703.
Moriwaki, K., and Chan, F. K. (2013). RIP3: a molecular switch for necrosis and inflammation. Genes Dev 27, 1640-1649.
Nagpal, K., Plantinga, T. S., Sirois, C. M., Monks, B. G., Latz, E., Netea, M. G., and Golenbock, D. T. (2011). Natural loss-of-function mutation of myeloid differentiation protein 88 disrupts its ability to form Myddosomes. J Biol Chem 286, 11875-11882.
O'Neill, L. A., Kishton, R. J., and Rathmell, J. (2016). A guide to immunometabolism for immunologists. Nat Rev Immunol 16, 553-565.
Ou, Y. H., Torres, M., Ram, R., Formstecher, E., Roland, C., Cheng, T., Brekken, R., Wurz, R., Tasker, A., Polverino, T., et al. (2011). TBK1 directly engages Akt/PKB survival signaling to support oncogenic transformation. Mol Cell 41, 458-470.
Pandey, S., Kawai, T., and Akira, S. (2014). Microbial sensing by Toll-like receptors and intracellular nucleic acid sensors. Cold Spring Harb Perspect Biol 7, a016246.
Peisley, A., Wu, B., Yao, H., Walz, T., and Hur, S. (2013). RIG-I forms signaling-competent filaments in an ATP-dependent, ubiquitin-independent manner. Mol Cell 51, 573-583.
Pelgrom, L. R., van der Ham, A. J., and Everts, B. (2016). Analysis of TLR-Induced Metabolic Changes in Dendritic Cells Using the Seahorse XF(e)96 Extracellular Flux Analyzer. Methods Mol Biol 1390, 273-285.
Strickson, S., Emmerich, C. H., Goh, E. T. H., Zhang, J., Kelsall, I. R., Macartney, T., Hastie, C. J., Knebel, A., Peggie, M., Marchesi, F., et al. (2017). Roles of the TRAF6 and Pellino E3 ligases in MyD88 and RANKL signaling. Proc Natl Acad Sci USA 114, E3481-E3489.
Sun, L., Wang, H., Wang, Z., He, S., Chen, S., Liao, D., Wang, L., Yan, J., Liu, W., Lei, X., et al. (2012). Mixed lineage kinase domain-like protein mediates necrosis signaling downstream of RIP3 kinase. Cell 148, 213-227.
Tanaka, Y., and Chen, Z. J. (2012). STING specifies IRF3 phosphorylation by TBK1 in the cytosolic DNA signaling pathway. Sci Signal 5, ra20.
Tannahill, G. M., Curtis, A. M., Adamik, J., Palsson-McDermott, E. M., McGettrick, A. F., Goel, G., Frezza, C., Bernard, N. J., Kelly, B., Foley, N. H., et al. (2013). Succinate is an inflammatory signal that induces IL-1beta through HIF-lalpha. Nature 496, 238-242.
Troutman, T. D., Hu, W., Fulenchek, S., Yamazaki, T., Kurosaki, T., Bazan, J. F., and Pasare, C. (2012). Role for B-cell adapter for PI3K (BCAP) as a signaling adapter linking Toll-like receptors (TLRs) to serine/threonine kinases PI3K/Akt. Proc Natl Acad Sci USA 109, 273-278.
Vance, R. E., Isberg, R. R., and Portnoy, D. A. (2009). Patterns of pathogenesis: discrimination of pathogenic and nonpathogenic microbes by the innate immune system. Cell Host Microbe 6, 10-21.
Ve, T., Vajjhala, P. R., Hedger, A., Croll, T., DiMaio, F., Horsefield, S., Yu, X., Lavrencic, P., Hassan, Z., Morgan, G. P., et al. (2017). Structural basis of TIR-domain-assembly formation in MAL- and MyD88-dependent TLR4 signaling. Nat Struct Mol Biol 24, 743-751.
Wang, H., Sun, L., Su, L., Rizo, J., Liu, L., Wang, L. F., Wang, F. S., and Wang, X. (2014). Mixed lineage kinase domain-like protein MLKL causes necrotic membrane disruption upon phosphorylation by RIP3. Mol Cell 54, 133-146.
West, A. P., Brodsky, I. E., Rahner, C., Woo, D. K., Erdjument-Bromage, H., Tempst, P., Walsh, M. C., Choi, Y., Shadel, G. S., and Ghosh, S. (2011). TLR signalling augments macrophage bactericidal activity through mitochondrial ROS. Nature 472, 476-480.
Xia, Z. P., Sun, L., Chen, X., Pineda, G., Jiang, X., Adhikari, A., Zeng, W., and Chen, Z. J. (2009). Direct activation of protein kinases by unanchored polyubiquitin chains. Nature 461, 114-119.
Xie, X., Zhang, D., Zhao, B., Lu, M. K., You, M., Condorelli, G., Wang, C. Y., and Guan, K. L. (2011). IkappaB kinase epsilon and TANK-binding kinase 1 activate AKT by direct phosphorylation. Proc Natl Acad Sci USA 108, 6474-6479.
Yamamoto, M., Sato, S., Hemmi, H., Hoshino, K., Kaisho, T., Sanjo, H., Takeuchi, O., Sugiyama, M., Okabe, M., Takeda, K., et al. (2003). Role of adaptor TRIF in the MyD88-independent toll-like receptor signaling pathway. Science 301, 640-643.

### EQUIVALENTS

Those skilled in the art will recognize, or be able to ascertain using no more than routine experimentation, many equivalents to the specific embodiments of the invention described herein. Such equivalents are intended to be encompassed by the following claims.

1.-24. (canceled)

25. A synthetic gene comprising a polynucleotide sequence encoding a hybrid protein comprising:
a myeloid differentiation primary response 88 (MyD88) protein; and
a polypeptide comprising a pLxIS motif, wherein:
p is a hydrophilic residue;
L is leucine;
x is any residue;
I is isoleucine; and
L is leucine.

26. The synthetic gene of claim 25, wherein the pLxIS motif is a polypeptide fragment of a MAVS protein, a STING protein, or a TRIF protein.

27. The synthetic gene of claim 26, wherein the pLxIS motif is a polypeptide fragment of a MAVS protein.

28. The synthetic gene of claim 27, wherein the MAVS protein comprises the amino acid sequence of SEQ ID NO: 2.

29. The synthetic gene of claim 27, wherein the pLxIS motif comprises amino acids 438-442 of SEQ ID NO: 2 (DLAIS).

30. The synthetic gene of claim 26, wherein the pLxIS motif is a polypeptide fragment of a STING protein.

31. The synthetic gene of claim 30, wherein the STING protein comprises the amino acid sequence of SEQ ID NO: 6.

32. The synthetic gene of claim 30, wherein the pLxIS motif comprises amino acids 362-366 of SEQ ID NO: 6 (ELLIS).

33. The synthetic gene of claim 26, wherein the pLxIS motif is a polypeptide fragment of a TRIF protein.

34. The synthetic gene of claim 33, wherein the TRIF protein comprises the amino acid sequence of SEQ ID NO: 8.

35. The synthetic gene of claim 33, wherein the pLxIS motif comprises amino acids 206-210 of SEQ ID NO: 8 (NLEIS).

36. The synthetic gene of claim 25, wherein the polypeptide comprising the pLxIS motif comprises an amino acid sequence having at least 95% sequence identity to SEQ ID NO: 1, or fragment thereof.

37. The synthetic gene of claim 25, wherein the polypeptide comprising the pLxIS motif comprises amino acids 13-17 of SEQ ID NO: 1 (RLLIS).

38. The synthetic gene of claim 25, wherein the polypeptide comprising the pLxIS motif comprises the amino acid sequence of SEQ ID NO: 1.

39. The synthetic gene of claim 25, wherein the polypeptide comprising a pLxIS motif is fused N-terminally to the myeloid differentiation primary response 88 (MyD88) protein.

40. The synthetic gene of claim 25, wherein the polypeptide comprising a pLxIS motif is fused C-terminally to the myeloid differentiation primary response 88 (MyD88) protein.

41. A method for inducing cytokine and/or interferon expression in a cell, the method comprising:
administering the synthetic gene of claim 25 to the cell.

42. The method of claim 41, wherein the cell is a cancer cell or an immune cell.

43. A synthetic gene comprising a polynucleotide sequence encoding a hybrid protein comprising:
a myeloid differentiation primary response 88 (MyD88) protein; and
a polypeptide comprising a RIPK3 death inducing domain.

44. A method for inducing cell death in a cell, the method comprising:
administering the synthetic gene of claim 43 to the cell.
